(19) United States
(12) Patent Application Publication
(10) Pub. No.: US 2004/0110295 A1
(43) Pub. Date: Jun. 10, 2004

(54) Nucleic acid vectors

(75) Inventors: Juha Punnonen, Belmont, CA (US); Doris Apt, Sunnyvale, CA (US); Robert G. Whalen, Foster City, CA (US)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen, Inc., a Delaware corporation, Redwood City, CA (US)

(21) Appl. No.: 10/446,629

(22) Filed: May 28, 2003

Related U.S. Application Data
(60) Provisional application No. 60/384,002, filed on May 28, 2002.

(51) Int. Cl. C12Q 1/68; C07H 21/04; C12N 15/85

(52) U.S. Cl. 435/455; 435/6; 435/320.1; 536/23.1

(57) ABSTRACT

The invention relates to nucleic acid vectors useful for expression and production of polypeptides, compositions comprising vectors, and methods for the production and use of vectors and polypeptides.

### CROSS-REFERENCE TO RELATED APPLICATIONS

This application claims benefit of U.S. Provisional Patent Application No. 60/384,002, filed on May 28, 2002, which application is incorporated herein by reference in its entirety for all purposes.

### STATEMENT REGARDING FEDERALLY SPONSORED RESEARCH AND DEVELOPMENT

This invention was developed in part with Government support by a grant from the Defense Advanced Research Projects Agency (DARPA) (Grant No. N65236-99-1-5421). The Government may have certain rights in this invention.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates generally to nucleic acid vectors and expression vectors for expression of heterologous polypeptides, compositions and host cells comprising such vectors, and methods for using and producing such vectors.

### BACKGROUND OF THE INVENTION

Recombinant DNA technology has enabled the expression of foreign (heterologous) proteins in a variety of host cells, including eukaryotes. For example, it is now possible to employ a nucleic acid vector comprising selected control elements to direct a host cell to produce a heterologous protein(s) encoded by a heterologous nucleic acid(s) that has been cloned into the vector. The vector is introduced into such host cells and the host cells are subjected to conditions that facilitate transcription or expression of the heterologous nucleic acid, leading to the expression of the desired foreign protein. Expression vectors can be engineered to produce high levels of the heterologous protein(s) of interest. Such vectors are useful for recombinantly producing the protein of interest, particularly when the protein is not readily available in nature or isolation or purification of the protein from its natural source is difficult, or when the protein is a newly designed novel or non-naturally occurring protein.

Notably, however, many existing expression vectors are unsuitable for use in mammals, such as humans, in therapeutic, gene therapy or DNA vaccine applications, since they include one or more components that may induce an adverse immune response or an undesirable allergic response. Furthermore, some existing vectors comprise DNA sequences that bear homology to the human genome and thus the administration of such vectors to mammals, including humans, may pose a danger of chromosomal integration. There is a need for expression vectors that are able to express desired polypeptide(s) of interest in vivo in mammals, including humans, with no or minimal undesirable effects. In particular, there is a need for expression vectors effective and safe for use in humans and other mammals in DNA vaccination strategies, therapeutic and prophylactic treatment methods, and/or gene therapy strategies, where the vectors do not to induce unwanted allergic and/or other immune responses or other undesirable side effects. The present invention fulfills these and other needs, as will be apparent to those skilled in the art upon consideration of the drawings and detailed description of the invention.

### SUMMARY OF THE INVENTION

In one aspect, the present invention provides nucleic acid vectors that are capable of expressing heterologous or recombinant polypeptides in a wide range of cells, including eukaryotic cells. A nucleic acid sequence that codes for such a heterologous or recombinant polypeptide is inserted into a vector of the invention and expression is achieved by transfecting a desired host cell with the vector and culturing g the cell under appropriate conditions to promote expression of the polypeptide.

In another aspect, the invention provides DNA expression vectors having the ability to express or produce considerable levels of heterologous or recombinant peptide or polypeptides in mammalian cells.

In one aspect, the nucleic acids and vectors of the invention comprise an expression vector capable of expressing an exogenous polypeptide upon incorporation into said expression vector of a polynucleotide encoding said exogenous polypeptide.

In another aspect, the invention provides an isolated or recombinant nucleic acid comprising a polynucleotide sequence that has at least about 90% nucleic acid sequence identity to a polynucleotide sequence selected from the group of SEQ ID NOS:1, 2 and 5, or a complementary polynucleotide sequence thereof.

In another aspect, the invention provides an isolated or recombinant nucleic acid comprising a polynucleotide sequence that has at least about 90% nucleic acid sequence identity to the polynucleotide sequence of SEQ ID NO:3 or 4, or a complementary polynucleotide sequence thereof.

Also provided are nucleic acid vectors comprising at least one nucleic acid of the invention. Some such nucleic acid vectors comprise a promoter, wherein said vector further comprises a heterologous nucleic acid coding sequence that encodes at least one polypeptide, said heterologous nucleic acid coding sequence operably linked to the promoter.

In another aspect, the invention provides a nucleic acid vector comprising a polynucleotide sequence that hybridizes under at least stringent conditions over substantially the entire length of a polynucleotide sequence selected from the group of SEQ ID NOS:1-5, or a complementary polynucleotide sequence thereof.

In yet another aspect, the invention provides an isolated expression vector construct for the expression of a polypeptide in a mammalian cell, the expression vector comprising: (a) a first polynucleotide sequence having at least 90% nucleic acid sequence identity to a polynucleotide sequence selected from the group of SEQ ID NOS:1, 2, and 5, wherein said first polynucleotide comprises a promoter for expression of the polypeptide in a mammalian cell and a terminator signal sequence; and (b) a second polynucleotide sequence encoding the polypeptide, wherein said second nucleic acid sequence is operably linked to the promoter.

In another aspect, the invention includes a DNA vaccine vector comprising a nucleic acid vector of the invention that comprises at least one polynucleotide sequence encoding at least one antigen. In another aspect, the invention provides a DNA vaccine vector comprising a nucleic acid vector of the invention that comprises at least one polynucleotide sequence encoding at least one co-stimulatory polypeptide. In a particular aspect, the invention provides a DNA vaccine vector comprises at least one polynucleotide sequence encoding at least one co-stimulatory polypeptide and at least one polynucleotide sequence encoding at least one antigen.

In yet another aspect, the invention provides a method for expressing a polypeptide, comprising: (a) providing a cell comprising the vector of claim 44, said vector further comprising a polynucleotide coding sequence that encodes the polypeptide; and (b) culturing said cell under conditions suitable for expression of the polypeptide.

Also provided in a method of expressing a polypeptide, the method comprising: (a) introducing into a population of cells a nucleic acid of claim 1, which nucleic acid further comprises a polynucleotide sequence that encodes the polypeptide, said polynucleotide sequence operatively linked to a regulatory sequence effective to produce the encoded polypeptide; (b) culturing the cells in a culture medium to express the polypeptide.

In another aspect, the invention includes is a method of producing a polypeptide, the method comprising: (a) introducing into a population of cells an expression vector comprising the nucleic acid of claim 1, said nucleic acid further comprising a polynucleotide sequence that encodes the polypeptide, said polynucleotide sequence operatively linked to a promoter sequence within the nucleic acid to produce the encoded polypeptide; (b) administering the expression vector into a mammal; and (c) isolating the polypeptide from the mammal or from a byproduct of the mammal.

Also provided are monocistronic expression vectors comprising at least one nucleic acid vector of the invention, wherein the nucleic acid vector comprises at least one exogenous polynucleotide sequence encoding at least one exogenous polypeptide, the polynucleotide sequence being operably linked to a promoter. Also included are bicistronic expression vectors comprising at least one nucleic acid vector of the invention, wherein the nucleic acid vector comprises at least two exogenous polynucleotide sequences, each such polynucleotide sequence encoding at least one exogenous polypeptide and operably linked to a promoter. A terminator sequence is typically included in each such monocistronic or bicistronic vector.

In another aspect, the invention provides a method for inducing an immune response in a subject, comprising administering to the subject at least one nucleic acid of the invention, wherein said nucleic acid comprises a mammalian promoter sequence and further comprises a polynucleotide sequence encoding an antigenic polypeptide that is operatively linked to the mammalian promoter sequence, said nucleic acid being administered in an amount sufficient to induce an immune response by expression of the polypeptide.

In yet another aspect is provided a method for enhancing an immune response to an antigen in a subject, which comprises administering to the subject a nucleic acid vector of the invention, wherein the vector further comprises at least one polynucleotide sequence encoding an immunomodulatory or co-stimulatory polypeptide, such that the immune response induced in the subject by the antigen is enhanced by the expressed immunomodulatory polypeptide. The immunomodulatory or co-stimulatory polypeptide is expressed and enhanced the immune response in the subject induced by an antigen.

In another aspect, the invention provides a method of treating a disorder or disease in a mammal in need of such treatment, comprising administering to the subject a nucleic acid vector of the invention, where nucleic acid further comprises a polynucleotide sequence that encodes a polypeptide useful in treating said disorder or disease. The polynucleotide sequence encoding the polypeptide is operatively linked to a mammalian promoter sequence effective to produce the encoded polypeptide. The mammalian promoter sequence comprises a portion of the polynucleotide sequence of the nucleic acid vector. The nucleic acid vector is administered in an amount sufficient to produce an effective amount of the polypeptide to treat said disorder or disease.

The nucleic acids of the invention may comprise synthetic nucleic acids.

The invention also provides compositions comprising at least one nucleic acid of the invention as described herein and an excipient or carrier. Some such compositions are pharmaceutical compositions and the excipient or carrier is a pharmaceutically acceptable excipient.

In another aspect, the invention provides cells comprising at least one nucleic acid or vector of the invention described herein. Some such cells express a polypeptide encoded by the nucleic acid or vector. Also provided are host cells comprising at least one nucleic acid or vector of the invention. The nucleic acid or vector of the invention may further comprise one or more polylinkers for incorporating exogenous nucleotide sequences that encodes exogenous polypeptides of interest (e.g., antigens, co-stimulatory polypeptides, cytokines, adjuvants, etc.) for therapeutic or prophylactic treatment methods (e.g., treating viral diseases, cancers, etc.) or for gene therapy methods.

In yet another aspect, the invention provides pharmaceutical compositions comprising a pharmaceutically acceptable excipient and at least one nucleic acid or vector of the invention, which optionally further comprises at least one additional exogenous nucleic acid encoding an exogenous polypeptide of interest.

Additional aspects, features, and advantages of the invention are apparent from the description below.

### BRIEF DESCRIPTION OF THE FIGURES

FIG. 1 illustrates a plasmid map of the mammalian DNA expression vector pMaxVax10.1 (abbreviated “pMV10.1”), which comprises, among other things: (1) a human CMV (Towne or AD169 strain) promoter region; (2) a polylinker; (3) a polyadenylation (polyA) signal from the bovine growth hormone gene (BGH polyA signal); and (4) the prokaryotic origin of replication ColE1 (which promotes high copy number of the plasmid in E. coli) and a Kanamycin resistant gene for amplification in E. coli. The nucleotide sequence of this expression vector is shown in SEQ ID NO:1. The plasmid map indicates the positions of restriction sites located in the polylinker (BamH1, EcoRI, KpnI, Asp718, XbaI) and additional restriction sites (NotI, BglII, PmeI, DraIII, AscI, NgMol, NheI, EcoRV, BsrG1) located between the functional elements. Resulting fragment sizes after restriction digest and gel electrophoresis can be calculated from the positions given in brackets behind the respective restriction sites.

FIG. 2 shows a plasmid map of the mammalian DNA plasmid expression vector named “pMV10.1-shCMV,” which comprises, among other things: (1) a shuffled, chimeric CMV promoter (clone 6A8); (2) a polylinker; (3) a polyadenylation signal from the bovine growth hormone gene (BGH polyA), and (4) the prokaryotic replication origin ColE1 and a Kanamycin resistant gene for amplification in E. coli. The nucleotide sequence of this expression vector is shown in SEQ ID NO:2. The polynucleotide sequence of CMV promoter 6A8 is set forth in SEQ ID NO:8 in copending, commonly assigned PCT application Ser. No. 01/20,123, entitled “Novel Chimeric Promoters,” filed Jun. 21, 2001, which published with International Publication No. WO 02/00897. The plasmid map indicates the positions of restriction sites located in the polylinker (BamH1, EcoRI, KpnI, Asp718, XbaI) and additional restriction sites (NotI, BglII, PmeI, DraIII, AscI, NgMol, NheI, EcoRV, BsrG1) located between the functional elements. Resulting fragment sizes after restriction digest and gel electrophoresis can be calculated from the positions given in brackets behind the respective restriction sites.

FIG. 3 depicts a plasmid map of a mammalian DNA monocistronic expression vector, which comprises the pMaxVax10.1 plasmid vector and a polynucleotide sequence encoding a CD28 receptor binding protein (termed a “CD28 binding protein” or “CD28BP”) cloned in the unique restriction sites BamH1 and KpnI in the polylinker of the vector. The vector is designated “pMV10.1-CD28BP.” The nucleotide sequence of this expression vector is shown in SEQ ID NO:3. The plasmid map lists also the additional cloning sites of the polylinker (EcoRI, Asp718, KpnI, NotI) and additional restriction sites (NotI, BglII, PmeI, DraIII, AscI, NgMol, NheI, EcoRV, BsrG1). Resulting fragment sizes after restriction digest and gel electrophoresis can be calculated from the positions given in brackets behind the respective restriction sites.

FIG. 4 shows a plasmid map of a mammalian DNA bicistronic expression vector, which comprises pMaxVax10.1 expression vector and an additional CMV promoter positioned downstream of the first expression cassette (first expression cassette comprises the first CMV promoter, CD28BP-15 gene, and first BGH polyA, as in FIG. 3) cloned into the unique NgoMI site of pMaxVax10.1, a second transgene (a polynucleotide that encodes EpCAM cancer antigen) cloned into the unique AccI and NheI restriction sites, and a second BGH polyA cloned into the restriction sites AgeI and NheI. The vector includes two CMV promoters and is named “pMV10.1-CD28BP-EpCAM.” Each of the two CMV promoters may comprise WT human CMV promoter (such as, e.g., Towne AD169 strain) or shuffled, chimeric or mutant CMV promoter (the promoter may include an enhancer and/or intron A, such as the enhancer/intron A of human CMV (e.g., Towne strain) or a chimeric, shuffled, or mutant enhancer and/or intron A region) including any of those described in copending, commonly assigned PCT application Ser. No. 01/20,123, entitled “Novel Chimeric Promoters,” filed Jun. 21, 2001, published with Int'l Publ. No. WO 02/00897. The plasmid map lists also the additional cloning sites of the polylinker (EcoRI, Asp718, KpnI, NotI) and additional restriction sites (NotI, BglII, PmeI, DraIII, AscI, EcoRV, BsrG1). Resulting fragment sizes after restriction digest and gel electrophoresis can be calculated from the positions given in brackets behind the respective restriction sites.

FIG. 5 illustrates a mammalian DNA monocistronic expression vector (“pCMV-Mkan”) comprising: (1) a CMV promoter; (2) optionally including a cloning site comprising a 26-residue nucleotide sequence comprising EcoRI and KpnI recognition sites to facilitate EcoRI and KpnI restriction endonuclease cleavage, respectively, for cloning of a heterologous polynucleotide sequence (termed “stuffer nucleotide sequence”); (3) a BGH polyadenylation signal; and (4) a Kanamycin resistant gene sequence; and (5) the prokaryotic replication origin ColE1 for amplification in E. coli. The optional stuffer nucleotide sequence, which comprises 26 nucleotide residues, is shown in SEQ ID NO:13. Because the stuffer sequence comprises EcoRI and KpnI recognition sites, it allows for convenient insertion of a heterologous polypeptide- or peptide-encoding nucleotide sequence of interest (e.g., an antigen, adjuvant, co-stimulatory immunomodulatory polypeptide or the like). The stuffer nucleotide sequence is optional and need not be included in the vector. In some instances, the stuffer sequence is removed (but need not be) upon insertion of the heterologous nucleotide sequence. This stuffer sequence represents a nucleotide sequence that includes the initiator methionine codon (ATG). This stuffer sequence may be replaced in its entirety by a protein coding sequence, which includes the open reading frame encoding for the protein, including at least the initiation and termination codons, thereby allowing for-expression of the protein. The polynucleotide sequence of SEQ ID NO:4 represents the pCMV-Mkan vector with the additional stuffer nucleotide sequence (26 residues). The polynucleotide sequence of SEQ ID NO:5 represents the polynucleotide sequence of the pCMV-Mkan vector without the stuffer nucleotide sequence.

FIG. 6 illustrates the expression of two dengue virus antigens from the vector pMV10.1 in mammalian cells in vitro, analyzed by Western Blot. The genes coding for the viral DEN-3 and DEN-4 membrane (prM) and envelope (E) antigens (DEN-3 prM/E and DEN-4 prM/E) were inserted into the pMV 10.1 expression vector and transfected into human HEK 293 cells. The antigenic proteins expressed in the cell lysates (Ly) and the medium supernatants (SN) were separated by gel electrophoresis, blotted to nitrocellulose filters, and analyzed by Western Blot with DEN-3 and DEN-4 serotype specific antibodies. The results shown in FIG. 6 illustrate expression of the antigens using the pMV10.1 vector and demonstrate that the vector is useful as an expression vector for expression of a heterologous protein following insertion of the nucleotide sequence encoding the heterologous protein into the pMV10.1 vector.

FIG. 7 illustrates optical density (OD) values (y-axis) obtained following DEN-specific antibody induction in mouse serum using ELISA plates coated with DEN-1, DEN-2, DEN-3 and DEN-4 serotype specific antigens. Groups of mice were immunized with one of the following plasmid vectors: 1) pMV10.1 expression vector encoding the DEN-3 prM/envelope antigen (abbreviated “DEN-3 prM/E”); 2) pMV10.1 expression vector encoding the DEN-4 prM/envelope antigen (abbreviated “DEN-4 prM/E”); or 3) pMV10.1 expression vector alone, with no heterologous antigen-encoding polynucleotide sequence, which served as a control vector. On the x-axis is shown the particular antigen expressed by the administered pMV10.1 vector (or no antigen as for the pMV10.1 control). Serum was collected from the mice at day 90 and analyzed for DEN-specific antibody induction in ELISA plates coated with DEN-1, DEN-2, DEN-3 and DEN-4 serotype specific antigens. These results confirm the in vivo expression of each of the two wild-type dengue virus antigens, DEN-3 prM/E and DEN-4 prM/E, from a pMV10.1 vector into which the respective antigen has been cloned, as determined by antibody induction in mice and serum analyses by ELISA.

Dengue (DEN) viruses are known among flaviviruses as agents of disease in humans. Dengue viruses comprise four known distinct, but antigenically related serotypes, named Dengue-1 (DEN-1 or Den-1), Dengue-2 (DEN-2 or Den-2), Dengue-3 (DEN-3 or Den-3), and Dengue-4 (DEN-4 or Den-4). Dengue virus particles are typically spherical and include a dense core surrounded by a lipid bilayer. FIELDS VIROLOGY, supra.

The genome of a dengue virus, like other flaviviruses, typically comprises a single-stranded positive RNA polynucleotide. FIELDS VIROLOGY, supra, at 997. The genomic RNA serves as the messenger RNA for translation of one long open reading frame (ORF) as a large polyprotein, which is processed co-translationally and post-translationally by cellular proteases and a virally encoded protease into a number of protein products. Id. Such products include structural proteins and non-structural proteins. A portion of the N-terminal of the genome encodes the structural proteins—the C protein, prM (pre-membrane) protein, and E protein—in the following order: C-prM-E. Id. at 998. The C-terminus of the C protein includes a hydrophobic domain that functions as a signal sequence for translocation of the prM protein into the lumen of the endoplasmic reticulum. Id. at 998-999. The prM protein is subsequently cleaved to form the structural M protein, a small structural protein derived from the C-terminal portion of prM, and the predominantly hydrophilic N-terminal “pr” segment, which is secreted into the extracellular medium. Id. at 999. The E protein is a membrane protein, the C-terminal portion of which includes transmembrane domains that anchor the E protein to the cell membrane and act as signal sequence for translocation of non-structural proteins. Id. The E protein is the major surface protein of the virus particle and is believed to be the most immunogenic component of the viral particle. The E protein likely interacts with viral receptors, and antibodies that neutralize infectivity of the virus usually recognize the E protein. Id. at 996. The M and E proteins have C-terminal membrane spanning segments that serve to anchor these proteins to the membrane. Id. at 998.

FIG. 8 illustrates the immune response induced in vivo in mice following immunization of mice with a pCMV-Mkan vector encoding a hepatitis envelope antigen.

### DETAILED DESCRIPTION OF THE INVENTION

The present invention provides nucleic acids, nucleic acid vectors, expression vectors, and cells and compositions comprising such nucleic acids and vectors. In addition, the invention provides methods of making and using such nucleic acids, vectors, expression vectors, cells, and compositions, and polypeptides expressed from such nucleic acids, vectors, expression vectors, etc.

In one aspect, the present invention provides nucleic acid vectors that are capable of expressing heterologous or recombinant polypeptides in a wide range of cells, including eukaryotic cells. A nucleic acid sequence that codes for such a heterologous or recombinant polypeptide is cloned or inserted into a vector of the invention and expression or production of the polypeptide is achieved by transfecting a desired host cell with the vector and culturing the cell under appropriate conditions to promote expression of the polypeptide. Nucleic acid vectors of the invention are effective and safe for use in mammals, including humans.

One aim of the present invention is to provide a vector capable of effectuating expression in a cell, such as, e.g., a eukaryotic cell, of at least one heterologous nucleic acid (e.g., DNA) that encodes a peptide or protein of interest. In one aspect, a vector of the invention comprises a nucleic acid comprising a promoter and a terminator sequence (e.g., BGH polyadenylation signal; see, e.g., Hunt et al., In: Atlas of Protein Sequence and Structure, M. O. Dayhoff ed., National Biomedical Research Found., Washington, DC, Vol. 5, Supp. 2, pp. 113-139), and optionally an origin of replication (ori) region (e.g., a prokaryotic origin of replication, a eukaryotic origin of replication, or both) optionally a nucleotide sequence encoding a selection or selectable marker (which can be a coding nucleic acid in a restriction site) for selection in E. coli, optionally an initiation region, e.g., a translation initiation region and/or a ribosome binding site, and usually at least one restriction site for insertion of heterologous nucleic acid encoding the heterologous or exogenous protein. In one aspect, a selectable marker sequence, such as one encoding G418 or blasticidine, or hygromycin for selection in eukaryotic cells, can be included in the vector, but such a selection marker, since it expresses another heterologous protein, would require the vector to further include a promoter suitable for directing synthesis of the marker in eukaryotic cells, wherein the promoter is operably linked to such selection marker, and a polyA signal sequence (e.g., SV40) for proper expression and function in eukaryotes.

A eukaryotic mRNA codes for only one protein. During transcription, the 5′ends of eukaryotic mRNAs are blocked by the addition of methyl caps. After transcription, a poly(A) tail is added at the 3′ end of the eukaryotic mRNA. The mRNA is then typically transported through pores in the nuclear membrane into the cytoplasm where it is translated. A heterologous protein or peptide is normally either not produced by a host cell, or is produced only in limited amounts. A protein or peptide can be expressed and produced in detectable amount from a host cell culture transfected with a vector of the invention comprising a heterologous nucleotide sequence encoding the heterologous protein or peptide using known recombinant DNA technologies and genetic methods.

In one aspect, the invention provides a DNA expression vector having the ability to express or produce significant levels of at least one heterologous or recombinant peptide or polypeptide of interest in a mammalian cell or population of mammalian cells.

Nucleic acids and vectors of the invention are useful for expression of a heterologous nucleotide sequence that encodes a polypeptide of interest. Nucleic acid and vectors of the invention are useful as DNA vaccines, gene therapy strategies, and for a variety of therapeutic and/or prophylactic treatments and applications in which a polypeptide of interest is desired to be expressed in cells or administered to cells in vivo or in vitro. A wide variety of polypeptides can be expressed using nucleic acids or vectors of the invention, including proteins, small peptides, fusion proteins, functional or biological equivalents thereof, homologues, and fragments of polypeptides, proteins or peptides, and/or equivalents, analogs, or derivatives thereof.

Definitions

It is also to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific examples of appropriate materials and methods are described herein.

As used in this specification and the appended claims, the singular forms “a”, “an” and “the” are to be construed to cover both singular and plural referents unless the content or context clearly dictates otherwise. Thus, for example, reference to “polypeptide” includes two or more such polypeptides. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The headings provided in the description of the invention are included merely for convenience and are not intended to be limiting in the scope of the disclosure.

The terms “nucleic acid,” “polynucleotide,” “polynucleotide sequence,” and “nucleotide sequence” are used to refer to a polymer of nucleotides (A,C,T,U,G, etc. or naturally occurring or artificial nucleotide analogues), e.g., DNA or RNA, or a representation thereof, e.g., a character string, etc, depending on the relevant context. The terms “nucleic acid” and “polynucleotide” are used interchangeably herein; these terms are used in reference to DNA, RNA, or other novel nucleic acid molecules of the invention, unless otherwise stated or clearly contradicted by context. A given polynucleotide or complementary polynucleotide can be determined from any specified nucleotide sequence. A nucleic acid may be in single- or double-stranded form.

The terms “protein,” “polypeptide,” “amino acid sequence,” and “polypeptide sequence” are used to refer to a polymer of amino acids (a protein, polypeptide, etc.) or a character string representing an amino acid polymer, depending on context. The terms “protein,” “polypeptide,” and “peptide” are used interchangeably herein. Given the degeneracy of the genetic code, one or more nucleic acids, or the complementary nucleic acids thereof, that encode a specific amino acid sequence or polypeptide sequence can be determined from the amino acid or polypeptide sequence.

A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide or polypeptide that does not appear in nature, for example, a variant of a naturally-occurring polynucleotide or polypeptide, respectively, is recombinant. A recombinant polynucleotide or recombinant polypeptide may include one or more nucleotides or amino acids, respectively, from more than one source nucleic acid or polypeptide, which source nucleic acid or polypeptide can be a naturally-occurring nucleic acid or polypeptide, or can itself have been subjected to mutagenesis or other type of modification.

An “expression vector” is a nucleic acid construct or sequence, generated recombinantly or synthetically, with specific nucleic acid elements that permit transcription and/or expression of another nucleic acid in a host cell. An expression vector can be part of a plasmid, virus, or nucleic acid fragment. In one example, an expression vector is a DNA vector, such as a plasmid, that comprises at least one promoter sequence and at least one terminator sequence (e.g., BGH polyadenylation sequence), and optionally an origin of replication (ori) sequence, and optionally a selection or selectable marker sequence. Optionally, the expression vector may further comprise at least one nucleotide coding sequence of interest that codes for at least one polypeptide, wherein the at least one promoter sequence is operably linked with the at least one coding sequence. The term “expression” includes any step involved in the production of the polypeptide including, but not limited to, transcription, post-transcriptional modification, translation, post-translational modification, and/or secretion.

A “host cell” includes any cell type that is susceptible to transformation with a nucleic acid.

The term “nucleic acid construct” or “polynucleotide construct” typically refers to a nucleic acid molecule, either single- or double-stranded, which is isolated from a naturally occurring gene or which has been modified to contain segments of nucleic acids in a manner that would not otherwise exist in nature, or an artificially engineered nucleic acid sequence.

The term “control sequence” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. Typically, a control sequence includes a promoter and transcriptional and translational stop signals. Control sequences may be provided with linkers for the purpose of introducing specific restriction sites facilitating ligation of the control sequences with the coding region of the nucleotide sequence encoding a polypeptide.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally transcription termination signals. Typically, the expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “coding sequence” typically refers to a nucleotide sequence that encodes a polypeptide, domain or fragment of the polypeptide, or directly specifies the amino acid sequence of the polypeptide. A DNA coding sequence typically refers to a DNA sequence (including a double-stranded DNA sequence) that is transcribed into RNA and the RNA translated into a polypeptide in vivo when under the control of a suitable regulatory sequence, such as a promoter. The boundaries of a coding sequence are generally determined by an open reading frame, which usually begins with the ATG start codon at the 5′ amino terminus. A translation stop codon may be present at the 3′ carboxy terminus. A polyadenylation signal and transcription termination sequence may be positioned downstream of (toward the 3′ end or 3′ to) the coding sequence.

A “heterologous” nucleotide sequence, region or domain of a nucleic acid construct (e.g., DNA construct) is an identifiable nucleic acid segment within a larger nucleic acid molecule that is not found in association with the larger molecule in nature.

The term “encoding” refers to the ability of a nucleotide sequence to code for one or more amino acids. The term does not require a start or stop codon. An amino acid sequence can be encoded in any one of six different reading frames provided by a polynucleotide sequence and its complement.

The term “gene” broadly refers to any nucleic acid segment (e.g., DNA) associated with a biological function. Genes include coding sequences and/or regulatory sequences required for their expression. Genes also include non-expressed DNA nucleic acid segments that, e.g., form recognition sequences for other proteins (e.g., promoter, enhancer, or other regulatory regions). Genes can be obtained from a variety of sources, including cloning from a source of interest or synthesizing from known or predicted sequence information, and may include sequences designed to have desired parameters.

Generally, the nomenclature used hereafter and the laboratory procedures in cell culture, molecular genetics, molecular biology, nucleic acid chemistry, and protein chemistry described below are those well known and commonly employed by those of ordinary skill in the art. In accordance with the present invention, common recombinant DNA techniques, molecular biology techniques, molecular genetics, and microbiology techniques may be used by one of skill in the art. For example, techniques such as those described in Sambrook, Goeddel, supra, and CURRENT PROTOCOLS IN MOLECULAR BIOLOGY, F. M. Ausubel et al., eds., Current Protocols, a joint venture between Greene Publishing Associates, Inc. and John Wiley & Sons, Inc. (1994, supplemented through 1999) (hereinafter “Ausubel”), DNA CLONING: A PRACTICAL APPROACH, Vols. I-II (Glover ed. 1985); Animal Cell Culture (Freshney ed. 1986) may be used for recombinant nucleic acid methods, nucleic acid synthesis, cloning methods, cell culture methods, transfection and transformation, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references that are provided throughout this document. The procedures therein are believed to be well known to those of ordinary skill in the art and are provided for the convenience of the reader.

A “subsequence” or “fragment” is any portion of the entire sequence.

Numbering of an amino acid or nucleotide polymer corresponds to numbering of a selected amino acid polymer or nucleic acid when the position of a given monomer component (amino acid residue, nucleotide residue, etc.) of the polymer corresponds to the same residue position (or equivalent residue position) in a selected reference polypeptide or polynucleotide.

The term “isolated,” when applied to a nucleic acid or polypeptide, typically refers to a nucleic acid or polypeptide that (1) is produced (e.g., replicated or cloned) or exists in a cell and thereafter rendered at least substantially free of other cellular components, such as biomolecules (e.g., a nucleic acid or polypeptide that is rendered -essentially-free of such other cellular biomolecules by purification and/or enrichment of a composition containing the nucleic acid or polypeptide, respectively); (2) is the dominant component in a composition or preparation and which may be (though not necessarily) the only detectable in a composition or preparation; and/or (3) is rendered present in a desired (i.e., approximately set) amount in a particular composition by purification, enrichment, synthesis, or other suitable technique. In particular, an isolated nucleic acid usually refers a nucleotide sequence that is not immediately contiguous with one or more nucleotide sequences with which it is normally immediately contiguous (i.e., at the 5′ and/or 3′ end) in the sequence from which it is obtained and/or derived. For example, an isolated gene is separated from open reading frames that flank the gene and encode a protein other than the gene of interest. An isolated nucleic acid or polypeptide comprises at least about 70% or 75%, typically at least about 80% or about 85%, or preferably at least about 90%, 95%, or more of a composition or preparation (e.g., percent by weight or volume).

An isolated nucleic acid or polypeptide can be obtained by application of any suitable isolation technique. For example, an isolated polypeptide can be obtained by expressing a nucleic acid encoding the polypeptide in a host cell in a medium, such that the polypeptide is present, and isolating the polypeptide by separating the polypeptide from other cellular biomolecules (e.g., other cellular polypeptides, lipids, glycoproteins, nucleic acids, etc.). Alternatively, an isolated polypeptide can be obtained by synthesizing the polypeptide through chemical synthesis techniques under conditions and at levels where the synthesized polypeptide is either the dominant polypeptide species in a composition (e.g., a library of polypeptides) or at least present in a predominant concentration with respect to other polypeptides and biomolecules in the composition. A polypeptide isolated from a cell culture from which it is expressed can subsequently be mixed in a composition such that it is no longer the dominant polypeptide species in the composition. Nucleic acids may be similarly isolated by suitable techniques.

The invention provides compositions that exhibit essential homogeneity with respect to polypeptide and/or nucleic acid content, such that contaminant polypeptide or nucleic acid species cannot be detected in the composition by conventional detection methods. Purity and homogeneity are typically determined using analytical chemistry techniques, such as polyacrylamide gel electrophoresis or high performance liquid chromatography. The term “purified,” as applied to nucleic acids or polypeptides, generally denotes a nucleic acid or polypeptide that is essentially free from other components as determined by standard analytical techniques (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” Particularly, it means that the, nucleic acid or polypeptide is at least about 50% pure, usually at least about 75% or 80% pure, more preferably at least about 85% or 90% pure, and most preferably at least about 99% pure (e.g., percent by weight on a molar basis).

In a related sense, the invention provides methods of enriching compositions for such molecules. A composition is enriched for a molecule when there is a substantial increase in the concentration of the molecule after application of a purification or enrichment technique. A substantially pure polypeptide or polynucleotide will typically comprise at least about 55%, 60%, 70%, 80%, 90%, 95%, or at least about 99% percent by weight (on a molar basis) of all macromolecular species in a particular composition.

A “signal peptide” is an amino acid sequence that is translated in conjunction with a polypeptide. A signal peptide may direct such polypeptide to the secretory system.

“Substantially the entire length of a polynucleotide sequence” or “substantially the entire length of a polypeptide sequence” refers to at least about 50%, generally at least about 60%, 70%, or 75%, usually at least about 80% or 85%, and preferably at least about 90%, 91%, 92%, 93%, 94%, 95%, 96%, 97%, 98%, 99%, 99.5% or more of the length of a polynucleotide sequence or polypeptide sequence, respectively.

“Naturally occurring” as applied to an object refers to the fact that the object can be found in nature as distinct from being artificially produced by man. Non-naturally occurring as applied to an object means the object cannot be found in nature.

“Synthetic” in reference to an entity or object means an entity or object produced at least in part by an artificial process, in particular, an object not of natural origin.

A “variant” of a polypeptide refers to a polypeptide comprising a polypeptide sequence that differs in one or more amino acid residues from the polypeptide sequence of a parent or reference polypeptide, usually in at least about 1, 2, 3, 4, 5, 6, 7, 8, 9, 10, 11, 12, 13, 14, 15, 20, 23, 25, 30, 40, 50, 75, 100 or more amino acid residues. A polypeptide variant may differ from a parent or reference polypeptide by, e.g., deletion, addition, or substitution of one or more amino acid residues of the parent or reference polypeptide, or any combination of such deletion(s), addition(s), and/or substitution(s). A “variant” of a nucleic acid refers to a nucleic acid comprising a nucleotide sequence that differs in one or more nucleic acid residues from the nucleotide sequence of a parent or reference nucleic acid, usually in at least about 1, 2, 3, 4, 5, 6, 7, 8, 9, 10, 11, 12, 13, 14, 15, 17, 20, 21, 24, 27, 30, 33, 36, 39, 40, 45, 50, 60, 66, 75, 90, 100, 120, 150, 225 or more nucleic acid residues. A nucleic acid variant may differ from a parent or reference nucleic acid, by e.g., deletion, addition, or substitution of one or more nucleic acid residues parent or reference nucleic acid, or any combination of such deletion(s), addition(s), and/or substitution(s). For example, the sequence of a polypeptide variant may differ from the parent or reference polypeptide sequence by a substitution, deletion, or insertion of at least about 1 to 15 or more amino acid residues of the parent or reference polypeptide sequence. The sequence of a nucleic acid variant may differ from the parent or reference nucleic acid by a substitution, deletion, or insertion of at least about 1 to 50 or more nucleic acid residues of the parent or reference nucleic acid sequence, or, alternatively, by substitution, deletion, or insertion of appropriate codon(s) in the parent or reference nucleic acid sequence such that the resulting encoded polypeptide comprises an amino acid sequence that has been modified by amino acid deletion, substitution or insertion when compared to a reference or parent polypeptide sequence.

The term “subject” as used herein includes, but is not limited to, an organism, including mammals and non-mammals. A mammal includes, a human, non-human primate (e.g., baboon, orangutan, monkey), mouse, pig, cow, goat, cat, rabbit, rat, guinea pig, hamster, horse, monkey, and sheep. A non-mammal includes a non-mammalian invertebrate and non-mammalian vertebrate, such as a bird (e.g., a chicken or duck) or a fish.

An “immunogen” refers generally to a substance capable of provoking or altering an immune response, and includes, but is not limited to, e.g., immunogenic proteins, polypeptides, and peptides; antigens and antigenic peptide fragments thereof, nucleic acids having immunogenic properties or encoding polypeptides having such properties.

An “immunomodulator” or “immunomodulatory” molecule, such as an immunomodulatory polypeptide or nucleic acid, modulates an immune response. By “modulation” or “modulating” an immune response is intended that the immune response is altered. For example, “modulation” of or “modulating” an immune response in a subject generally means that an immune response is stimulated, induced, inhibited, decreased, increased, enhanced, or otherwise altered in the subject. Such modulation of an immune response can be assessed by means known to those skilled in the art, including those described below. An “immunostimulator” is a molecule, such as a polypeptide or nucleic acid, that stimulates an immune response.

An immune response generally refers to the development of a cellular or antibody-mediated response to an agent, including, e.g., an antigen, immunogen, an immunomodulator, immunostimulator, or nucleic acid encoding any such agent. An immune response includes production of at least one or a combination of cytotoxic T lymphocytes (CTLs), B cells, antibodies, or various classes of T cells that are directed specifically to antigen-presenting cells expressing the antigen of interest.

An “antigen” refers to a substance that is capable of inducing an immune response (e.g., humoral and/or cell-mediated) in a host, including, but not limited to, eliciting the formation of antibodies in a host, or generating a specific population of lymphocytes reactive with that substance. Antigens are typically macromolecules (e.g., proteins and polysaccharides) that are foreign to the host.

An “adjuvant” refers to a substance that enhances an immune response. For example, an adjuvant may enhance an antigen's immune-stimulating properties or the pharmacological effect(s) of a compound or drug. An adjuvant may comprise an oil, emulsifier, killed bacterium, aluminum hydroxide, or calcium phosphate (e.g., in gel form), or any combination of one or more thereof. Examples of adjuvants include “Freund's Complete Adjuvant,” “Freund's incomplete adjuvant,” Alum, and the like. Freund's Complete Adjuvant is an emulsion of oil and water containing an immunogen, an emulsifying agent and mycobacteria. Freund's Incomplete Adjuvant is the same, but without mycobacteria. Other adjuvants include BCG adjuvants, DETOX, and haptens, such as dinitrophenyl (DNP). An adjuvant is typically administered to a subject (e.g., via injection intramuscularly or subcutaneously) in an amount sufficient to enhance an immune response.

A “pharmaceutical composition” refers to a composition suitable for pharmaceutical use in a subject, including an animal or human. A pharmaceutical composition typically comprises an effective amount of an active agent and a carrier. The carrier is typically pharmaceutically acceptable carrier.

An “effective amount” means a dosage or amount of a molecule or composition sufficient to produce a desired result. The desired result may comprise an objective or subjective improvement in the recipient of the dosage or amount. For example, the desired result may comprise a measurable or testable induction, promotion, enhancement or modulation of an immune response in a subject to whom a dosage or amount of a particular antigen or immunogen (or composition thereof) has been administered. An amount of an immunogen sufficient to produce such result also can be described as an “immunogenic” amount.

A “prophylactic treatment” is a treatment administered to a subject who does not display signs or symptoms of, or displays only early signs or symptoms of, a disease, pathology, or disorder, such that treatment is administered for the purpose of preventing or decreasing the risk of developing the disease, pathology, or disorder. A prophylactic treatment functions as a preventative treatment against a disease, pathology, or disorder. A “prophylactic activity” is an activity of an agent that, when administered to a subject who does not display signs or symptoms of, or who displays only early signs or symptoms of, a pathology, disease, or disorder, prevents or decreases the risk of the subject developing the pathology, disease, or disorder. A “prophylactically useful” agent refers to an agent that is useful in preventing or decreasing development of a disease, pathology, or disorder.

A “therapeutic treatment” is a treatment administered to a subject who displays symptoms or signs of pathology, disease, or disorder, in which treatment is administered to the subject for the purpose of diminishing or eliminating those signs or symptoms. A “therapeutic activity” is an activity of an agent that eliminates or diminishes signs or symptoms of pathology, disease or disorder when administered to a subject suffering from such signs or symptoms. A “therapeutically useful” agent means the agent is useful in decreasing, treating, or eliminating signs or symptoms of a disease, pathology, or disorder.

An “epitope” refers to an antigenic determinant capable of specific binding to a part of an antibody. Epitopes usually consist of chemically active surface groupings of molecules such as amino acids or sugar side chains and usually have specific 3-dimensional structural characteristics, as well as specific charge characteristics. An epitope may comprise a short peptide sequence (e.g., 3-20 amino acid residues). Conformational and nonconformational epitopes are distinguished in that the binding to the former but not the latter is lost in the presence of denaturing solvents.

A “specific binding affinity” between two molecules, e.g., a ligand and a receptor, means a preferential binding of one molecule for another. The binding of molecules is typically considered specific if the binding affinity is about 1×10² M⁻¹ to about 1×10⁹ M⁻¹ (i.e., about 10⁻²-10⁻⁹ M) or greater.

A nucleic acid is “operably linked” with another nucleic acid sequence when it is placed into a functional relationship with another nucleic acid sequence. For instance, a promoter or enhancer is operably linked to a coding sequence desired to be expressed if it increases the transcription of the coding sequence and/or is capable of directing the replication and/or expression of the coding sequence for all or part of the protein that is desired to be expressed. Operably linked nucleic acid sequences may be contiguous and, where necessary to join two protein coding regions, contiguous and in reading frame. However, since enhancers generally function when separated from the promoter by several kilobases and intronic sequences may be of variable lengths, some nucleic acid sequences may be operably linked, but not contiguous.

A “cytokine” includes, e.g., interleukins, interferons, chemokines, hematopoietic growth factors, tumor necrosis factors and transforming growth factors. In general these are small molecular weight proteins that regulate maturation, activation, proliferation, and differentiation of cells of the immune system.

The term “screening” describes, in general, a process for identification for molecules of interest or cells comprising such molecules. Several properties of the respective molecules or cells comprising such molecules can be used in selection and screening, for example, an ability of a respective molecule to induce an immune response in a test system or resistance of cells to a particular antibiotic. Selection is a form of screening in which identification and physical separation are achieved simultaneously by expression of a selection marker, which, in some genetic circumstances, allows cells expressing the marker to survive while other cells die (or vice versa). Screening markers include, for example, luciferase, beta-galactosidase and green fluorescent protein, reaction substrates, and the like. Selection markers include drug, antibiotic and toxin resistance genes, and the like. Because of limitations in studying primary immune responses in vitro, in vivo studies are particularly useful screening methods.

The term “homology” generally refers to the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar, but are derived or evolved from different or the same species using the techniques described herein.

Various additional terms are defined or otherwise characterized herein.

Nucleic Acids and Vectors of the Invention

In one aspect, the invention provides a nucleic acid vector capable of expressing one or more heterologous polypeptides of interest, where the nucleotide sequence coding for the polypeptide has been inserted or incorporated into the nucleotide sequence of the vector. A vector of the invention is capable of expressing one or more heterologous polypeptide-encoding nucleotide sequences in a particular host cell, such as a eukaryotic cell, including, e.g., but not limited to, a mammalian cell. Mammalian cells include, e.g., but are not limited to, primate, murine, bovine, rodent, Chinese hamster ovary, and human cells.

Vectors of the present invention may be in the form of DNA plasmids, which are circular double-stranded DNA constructs. The vector can be, e.g., an expression vector, a cloning vector, a packaging vector, or the like. The invention also includes a cell transduced or transfected by the vector.

As discussed in greater detail below, in one aspect, the vector is a monocistronic vector comprising one heterologous or exogenous nucleotide sequence encoding a heterologous or exogenous polypeptide of interest that is operably linked to a promoter sequence in the vector. Alternatively, the vector is bicistronic vector comprising two heterologous nucleotide sequences, each of which encodes a polypeptide of interest and each of which is operably linked to a promoter.

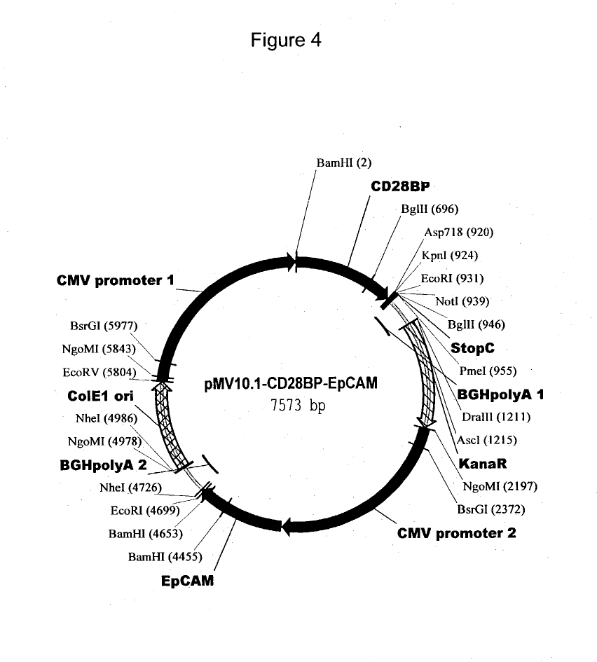

Vectors of the invention comprise at least one promoter for efficient transcription of a heterologous or exogenous nucleotide sequence encoding a peptide or protein on interest in eukaryotic cells in vitro and in vivo. The promoter may comprise a human CMV promoter and optionally includes the enhancer and/or intron A from human CMV. Alternatively, the promoter is a chimeric, shuffled or mutant CMV promoter (examples of which are provided in copending, commonly assigned PCT application Ser. No. 01/20,123, filed Jun. 21, 2001, which published with International Publication No. WO 02/00897. The two promoters present in a bicistronic vector of the invention (see, e.g., FIG. 4) may be the same or different. Different promoters may be selected for their dissimilar transcription abilities. For example, in a therapeutic bicistronic vector comprising a first expression cassette that includes a heterologous antigen-encoding polynucleotide sequence and a second expression cassette that comprises a heterologous immunomodulatory polypeptide-encoding polynucleotide sequence, it may be desirable to modulate expression of both heterologous sequences by employing a strong promoter for enhanced expression of the antigen and a weaker promoter for a more moderate expression of the immunomodulatory polypeptide. The weaker promoter may be, e.g., a wild-type human CMV (Towne or AD 169 strain), and the stronger promoter may be a chimeric CMV promoter shown to enhance exogenous protein expression, such as a strong chimeric CMV promoter shown in copending, commonly assigned PCT application Ser. No. 01/20,123, filed Jun. 21, 2001, which published with International Publ. No. WO 02/00897. Alternatively, the stronger promoter may be a wild-type human CMV (e.g., Towne or AD169 strain), and the weaker promoter may be a chimeric CMV promoter shown-to-enhance exogenous protein expression; weaker chimeric CMV promoters are described in WO 02/00897.

Expression vectors of the invention also typically comprise at least one terminator nucleotide sequence. Typically the terminator sequence is one that is appropriate for expression in mammalian cells, such as BGH poly A signal.

Expression vectors of the invention optionally comprise at least one prokaryotic origin of replication and at least one nucleic acid sequence encoding a selectable marker for selection in E. coli. The origin of replication is typically the ColE1 origin of replication. The selectable or selection marker is one that allows for phenotypic selection in transfected or transformed host cells. Although various markers can be employed for selection in E. coli, expression vectors of the present invention typically comprise at least one selection or selectable marker that would not produce a translation product (as, e.g., during replication of the vector in E. coli) that would cause an undesirable effect in a mammal, such as a human, to whom the vector is administered in a therapeutic application. The selection or selectable marker is typically an antibiotic resistance gene marker. Given that some antibiotic resistance markers (such as ampicillin and tetracycline resistance gene markers) have the potential to induce undesirable allergic or immune responses in mammals, especially humans, an expression vector of the invention typically comprises kanamycin resistance gene marker or other similar marker that is unlikely to induce such unwanted effects. For example, with production (which may include amplification and isolation) of an expression vector comprising an ampicillin or tetracycline marker in bacterial cells (e.g., E. coli) as in many standard manufacturing processes, there is a risk of production of some of the antibiotic and thus possible contamination of the produced vector with the antibiotic. Such potentially contaminated vectors might cause responses in antibiotic-sensitive individuals upon administration. In contrast, vectors of the present invention include a antibiotic selectable marker that avoids this problem (e.g., kanamycin resistant gene selectable marker). Thus, vectors of the invention can be amplified in bacteria (e.g., E. coli), purified therefrom and subsequently administered to mammals, including humans, without concern for contaminant antibiotics to which some such mammals (humans) might be sensitive.

Expression vectors of the invention comprising at least one promoter sequence that brings about efficient transcription and translation of at least one inserted heterologous DNA sequence are used in connection with one or more particular types of :host cells. In one aspect of the invention, the expression vector comprises a promoter(s) and terminator(s), and optionally an origin of replication, and optionally at least one specific nucleotide sequence capable of providing phenotypic selection for host cells carrying the expression vector. For expression in mammalian cells, the vector. The expression vector may be introduced into more than one type of host cell. To facilitate expression of a desired nucleotide sequence(s) or gene(s) in a particular host cell(s), or replication of the vector in a particular host cell(s), a suitable promoter sequence(s) and/or origin(s) of replication may be required for the particular type of host cell. As noted above, a wild-type human CMV promoter (Towne or AD169 strain) or s chimeric, mutant or shuffled CMV promoter is preferably employed for expression in mammalian cells, including primate and human cells. Optimal cell growth may be achieved by culturing cells transfected or transformed with the vector by methods well known in the art. Often, E. coli cells are used as host cells to prepare and acquire sufficient amounts of a vector, such as a plasmid. Notably, host cells used to make sufficient quantities of the vector may differ from host cells in which the vector is used (e.g., mammalian cells, human tissue, etc.), such as for therapeutic applications.

Vectors of the invention can be introduced into host cells by a variety of methods well known to those skilled in the art. See e.g., Sambrook, Goeddel. Host cells can be transfected with one or more expression vectors of the invention by electroporation, gene-gun delivery, injection, or by any transfection facilitating material, including, e.g., transfection-facilitating viral particles, lipid formulations, liposomal formulations, and/or charged lipids, as is discussed in greater detail below.

The vectors of the invention are useful for expressing a variety of heterologous polynucleotide coding sequences, such as a polynucleotide coding sequence that encodes a polypeptide or peptide of interest, in eukaryotic cells. Suitable eukaryotic cells in which vectors of the invention may be introduced for expression of the heterologous polynucleotide sequence include, e.g., mammalian cells of any type, such as primate, bovine, murine and human cells.

The heterologous polypeptide-encoding polynucleotide that is to be expressed in cells in vivo, ex vivo, or in vitro is not limited to any particular polynucleotide coding for any particular polypeptide. Polynucleotides coding for a large number of physiologically active peptides and antigens or immunogens are known in the art and can be readily obtained by those of skill in the art. A nucleotide sequence encoding one or more of a wide variety of polypeptides of interest that are desired to be expressed can be incorporated into a vector of the invention. Examples of polypeptides that can be expressed include, but are not limited to, e.g., antigens, immunomodulatory polypeptides, adjuvants, fusion proteins, epitopes, co-stimulatory polypeptides, cytokines, chemokines, antigens (including, e.g., but not limited to, cancer or tumor antigens, viral antigens, allergens, bacterial antigens, food allergens, etc.), antigenic determinants, immune-stimulating molecules, and adjuvants, and agents and components suitable for DNA vaccination and/or gene therapy, etc. The expression vectors of the invention systems are also advantageous in that they can be used for the expression and production of any modified proteins, such a artificially created mutants or recombinant, chimeric, or shuffled proteins, including those that have improved properties over their wild-type counterpart, that differ from wild-type proteins, can be created to further simplify the purification of the resultant protein. When cloned into the vector, a polynucleotide coding sequence that encodes part or all of a polypeptide that is desired to be expressed is operably linked to the promoter (optionally to an enhancer and/or intron or other regulatory sequence).

Following introduction into host cells, expression of the heterologous polypeptide is typically achieved by culturing the host cells under suitable known conditions that allow for polypeptide expression. Those of skill in the art can readily determine appropriate culture conditions suitable for culturing of a particular host cell.

In one aspect of the invention, host cells comprising at least one vector of the invention can be identified and selected by the presence or absence of resistance to the antibiotic, kanamycin, which is expressed from the kanamycin resistance marker gene incorporated into the vector. For example, selection for cells containing recombinant DNA molecules can be made by growth in the presence of the antibiotic. Expression of the marker gene sequence in an E. coli cell indicates the presence of at least one vector of the invention. Cell(s) comprising at least one such vector can be selected.

Host cells comprising an expression vector that includes a heterologous polypeptide-encoding polynucleotide sequence can be identified and/or selected by a variety of known assays, depending upon the type and nature of the heterologous polypeptide that is desired to be expressed. For example, standard immunological assays (e.g., Western blot techniques, ELISA assays) or enzymatic activity assays can be used to detect the presence or production of the heterologous polypeptide. Standard hybridization techniques to identify or detect the presence of nucleic acid encoding the heterologous polypeptide in host cells using nucleic acid probes complementary to the nucleic sequence encoding the heterologous polypeptide (e.g., DNA-DNA hybridization or DNA-RNA hybridization). One skilled in the art can define appropriate hybridization conditions. See, e.g., Sambrook. Additionally, all of the nucleic acids in the host cells can be removed from the cells and detected or identified by hybridization to such probes. The presence of relative amounts of heterologous mRNA corresponding to the heterologous polypeptide or fragment(s) thereof can also be determined by hybridization assays using standard Northern blot assay and RNA probes complementary to the mRNA sequence in according with common Northern hybridization techniques known to those skilled in the art. Conventional Southern blot hybridization techniques may also be employed to assess the presence and copy number of genes and nucleic acids; such techniques are known to persons of skill in the art.

The expression vectors of the invention can be conveniently amplified and isolated from host cells (including, e.g., bacterial cells (e.g., E. coli) which are typically employed for vector amplification and manufacturing) using techniques well known to those of skill in the art. See, e.g., Sambrook. Ausubel, and Goeddel, all supra.

Vectors of the present invention may further comprise additional polynucleotide sequences (e.g., DNA sequences) known to those of skill in the art that have particular functions. For example, vectors of the invention may include one or more signal sequences or secretory sequences for proper and efficient secretion of the expressed protein, one or more nucleotide sequences corresponding to one or more restriction sites for cleavage of the vector at particular locations by restriction endonucleases, nucleotide sequences that enhance stability of the vector.

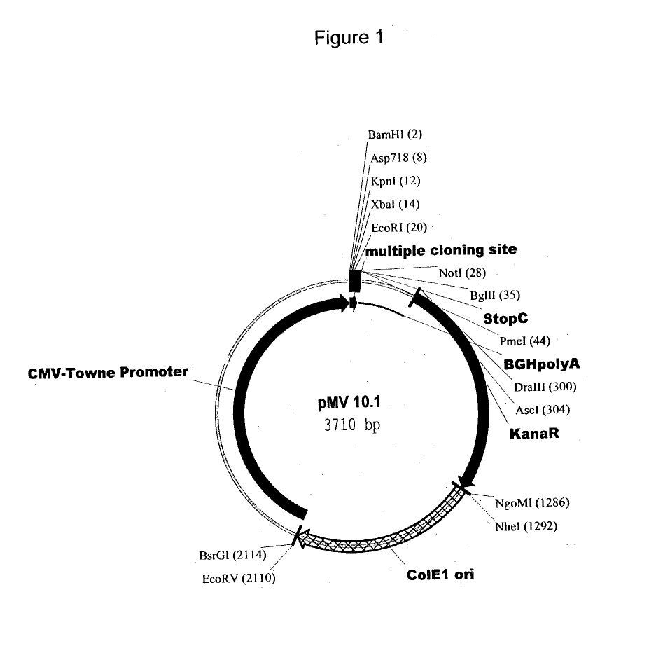

In one aspect, the invention provides an expression vector that comprises: 1) a Col E1 origin of replication (which promotes a high copy number of the plasmid in the E. coli recipient cells); 2) a kanamycin resistance gene marker; 3) a CMV promoter, preferably a human CMV Towne or AD169 promoter (optionally also including the enhancer and/or intron A of human CMV) or a shuffled or chimeric CMV promoter (optionally also including the enhancer and/or intron A of human CMV), as described further below; 4) a terminator sequence, such as BGH polyadenylation (polyA) signal sequence (the transcription of this sequence into messenger RNA (mRNA) is capable of signaling polyadenylation, which is the addition of a tail or long chain of adenine-containing nucleotides); and 5); at least one restriction site and typically a region of multiple restriction sites to facilitate the cloning of exogenous one or more polynucleotides or genes to be expressed. Exemplary vectors are shown in FIGS. 1, 2, and 5.

In another aspect, the invention provides a vector that comprises a CMV promoter followed by (i.e., upstream of or toward the 5′ end) a cloning site for cloning of at least one heterologous polynucleotide sequence of interest to be expressed, which is followed by a Stop Codon (“StopC”), which provides a signal to stop transcription of the inserted heterologous gene and prevents improper read through, such as improper transcription of the kanamycin resistance gene sequence (“KanaR”), SV40 or BGH polyadenylation signal nucleotide sequence which terminates translation (and provides a polyadenylation sequence), a kanamycin resistance gene selectable marker sequence (e.g., for selection of the vector in bacteria, e.g., E. coli), and a ColE1 origin of replication. A heterologous (foreign) polynucleotide sequence (e.g., cDNA) can be cloned into the vector between various restriction endonuclease sites (for example, 5 such sites are shown in FIG. 1) efficiently in the proper orientation using techniques well known in the art.

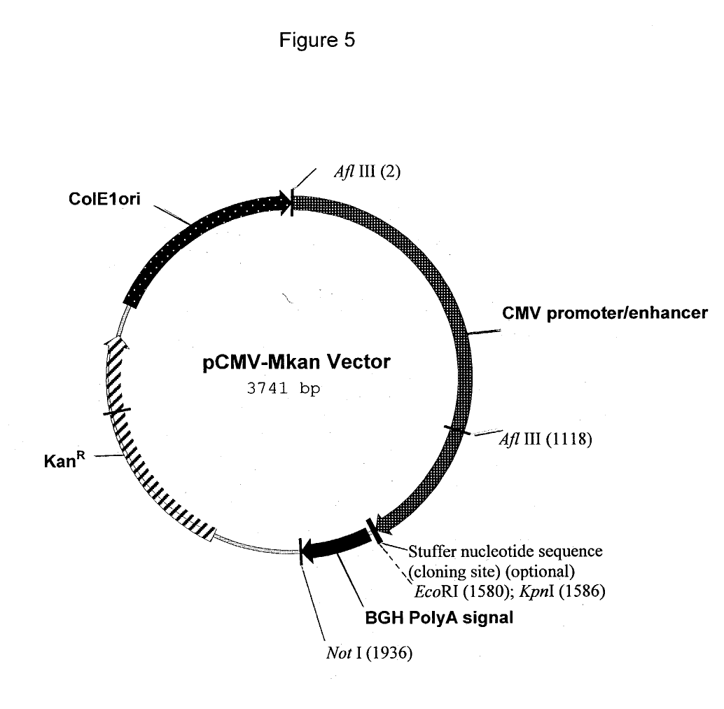

In yet another aspect, vectors of the invention comprise a human CMV promoter, e.g., Towne or AD169 strain (and optionally including the enhancer and/or intron A of human CMV) followed by (i.e., downstream of) a cloning site for cloning of at least one heterologous polynucleotide sequence of interest to be expressed. In one embodiment, the cloning site comprises a nucleotide sequence of 26 residues that includes EcoRI and KpnI recognition sites (see SEQ ID NO:13). This cloning site, which is termed the “stuffer nucleotide sequence,” is followed by a first intervening nucleotide sequence segment, which is then followed by a BGH polyA or SV40 polyA signal nucleotide sequence for termination of translation. The polyA sequence is followed a second intervening nucleotide sequence segment, which is followed by a kanamycin resistance marker gene sequence (Kan^{R} or Kana^{R}), which is then followed by a third intervening nucleotide sequence segment. Following the third intervening nucleotide sequence segment is a ColE1 origin of replication. A heterologous polynucleotide sequence can be cloned into the vector between various restriction endonuclease sites (5 such sites are shown in FIG. 1; see also FIG. 5) efficiently in the proper orientation using techniques well known in the art.

It will be apparent to those of ordinary skill in the art that various substitutions and/or modifications can be made to the invention disclosed herein, including, e.g., the vectors, compositions and methods described herein, without departing from the scope and spirit of the invention.

In one aspect, vectors of the invention include at least one heterologous coding nucleic acid that is inserted into the vector in at least one restriction site of the vector. The at least one nucleotide sequence cloned into the vector typically encodes at least one peptide or polypeptide of interest. In one embodiment, the heterologous sequence is a heterologous DNA sequence that encodes a therapeutic polypeptide or peptide or interest, or alternatively or in addition, a marker or tag, e.g., a Histidine tag. As noted above, the polypeptide or peptide may comprise, but is not limited to, e.g., at least one antigen, epitope, immunomodulatory polypeptide, chemokine, cytokine, adjuvant, fusion protein, or the like, or any combination thereof. Methods for cloning such nucleotide sequence into a vector of the invention are well known. See, e.g., Sambrook, et al, MOLECULAR CLONING, A LABORATORY MANUAL (3rd Ed., Cold Spring Harbor Laboratory Press, 2001) (hereinafter “Sambrook”); METHODS IN ENZYMOLOGY, Vol. 185, “Gene Expression Technology,” (David V. Goeddel ed., Academic Press, Harcourt Brace Jovanovich, Publishers, 1991) (hereinafter “Goeddel”). Vectors expressing at least one such polypeptide or peptide can be readily constructed and transformed into cells by one of ordinary skill in the art using teachings disclosed in, e.g., Sambrook, Goeddel, or other methods known in the art. Furthermore, the encoded polypeptide or peptide can be expressed and detected art using teachings disclosed in, e.g., Sambrook, Goeddel, or other methods known in the art. Suitable methods provided in Sambrook, Goeddel, or other known methods can be appropriately modified, if desired, by one of ordinary skill in the art without undue experimentation. The nucleotide sequence encoding the peptide or polypeptide of interest to be expressed (e.g., peptide- or polypeptide-coding sequence) is inserted or cloned into the vector of the invention in a suitable relationship to the promoter and other transcriptional regulatory sequences of the vector and in the correct reading frame so that the heterologous peptide or polypeptide, respectively, is properly produced.

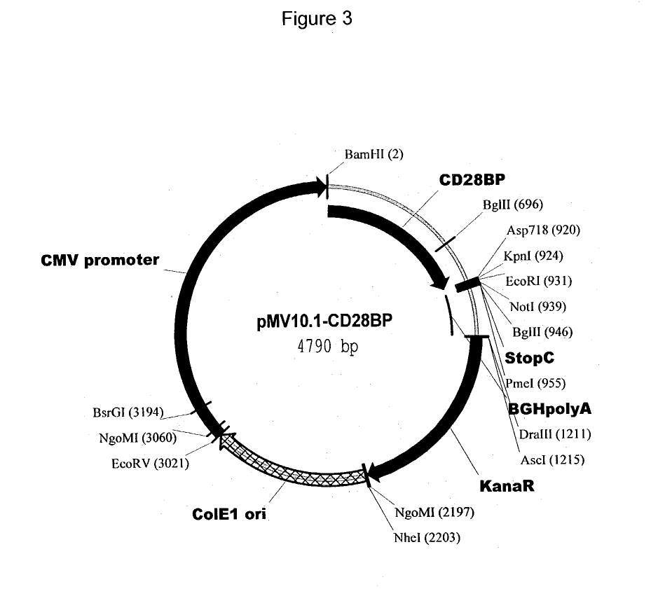

For example, at least one heterologous coding sequence encoding a polypeptide of interest can be cloned into at least one restriction site of the vectors showing in any of FIGS. 1, 2, and 5, such as the pMV10.1 vector (FIG. 1) or pCMV-Mkan vector (FIG. 5). An exemplary monocistronic vector of the invention comprising a heterologous polynucleotide sequence encoding a co-stimulatory polypeptide (e.g., a polypeptide that binds human CD28 receptor) is shown in FIG. 3. An exemplary bicistronic vector of the invention comprising a first heterologous polynucleotide sequence encoding a co-stimulatory polypeptide (e.g., a polypeptide that binds human CD28 receptor) and a second heterologous polynucleotide sequence encoding a human EpCAM/KSA antigen is shown in FIG. 4. Each of the first and second polynucleotide sequences is operably linked to a promoter.

In one aspect, the invention provides a DNA vaccine comprising an expression vector of the invention (e.g., pMV10.1 (SEQ ID NO:1) or pCMV-Mkan (see, e.g., SEQ ID NO:5)) into which has been inserted at a designated cloning site at least one polynucleotide sequence encoding at least one polypeptide of interest. The pMV10.1 vector includes a multiple cloning site downstream of the CMV promoter. The pCMV-Mkan includes the stuffer nucleotide sequence cloning site (that includes EcoRI and KpnI recognition sites) positioned downstream of the CMV promoter. The stuffer nucleotide sequence serves as a placement holder for insertion of a heterologous polynucleotide sequence into the vector at the proper position. Upon insertion of the heterologous polypeptide-encoding polynucleotide sequence into the vector, the stuffer nucleotide sequence may optionally be removed, if desired.

The expression vectors of the invention, including e.g., pMaxVax10.1 (“pMV10.1”) and pCMV-Mkan vector, were designed for use in the development of DNA vaccines and therapies, including gene therapies, for humans and ultimately for use in humans as DNA vaccines or in treatment protocols. The expression vectors of the invention were also particularly designed for use as DNA vaccines or therapeutic plasmid vehicles for delivery of therapeutic proteins to mammals, especially humans. For example, a polynucleotide sequence encoding at least one polypeptide of interest (e.g., antigen, adjuvant or immunomodulatory polypeptide) can be inserted into the vector at the appropriate cloning site and administration of the vector to a subject would result in expression of the polypeptide of interest.

In particular, the pMV10.1 and pCMV-Mkan expression vectors were designed to be consistent with the Food and Drug Administration (FDA) document, Points to Consider on Plasmid DNA Vaccines for Preventive Infectious Disease Indications (Docket no. 96N-0400). DNA sequences with possible homology to the human genome were limited to minimize the possibility of chromosomal integration. The pMV10.1 vector is 3710 base pairs in length and comprises: (i) a human cytomegalovirus (hCMV) Towne strain immediate-early promoter, including the human CMV enhanced and intron A, for high-level expression in mammalian cells, (ii) a bovine growth hormone (BGH) polyadenylation signal for efficient transcriptional termination and polyadenylation of mRNA, (iii) a Kanamycin resistance gene for efficient selection in E. coli (and to ensure no or minimal undesirable allergic or other immune response(s) or side effects in humans, as may observed with expression vectors comprising ampicillin or tetracycline gene markers), and (iv) the ColE1 origin of replication from pUC for high-copy number replication in E. coli. The pMV 10.1 vector also contains a polylinker (with restriction sites for BamHI, Asp718, KpnI, EcoRI, NotI, and BglII) for cloning of one or more antigens to be expressed, and additional restriction sites (PmeI, DraIII, AscI, NgoMI, NheI, BsrGI, and RV) located between the above listed functional elements (see FIG. 1). The nucleotide sequence of the pMV10.1 vector is shown in SEQ ID NO:1. Within the polynucleotide sequence of SEQ ID NO:1, the polylinker sequence comprises nucleotide residues 1-48; the BHG polyA sequence comprises nucleotide residues 48-293; the Kanamycin resistant gene sequence comprises nucleotide residues 303-1284; the ColE1 origin of replication comprises nucleotide residues 1291-2106; and the human CMV (Towne) promoter/enhancer/Intron A comprises nucleotide residues 2113-3710. A nucleotide sequence encoding an exogenous polypeptide of interest is typically cloned into BamHI and EcoRI of the polylinker.

The pCMV-Mkan vector was similarly designed for the development of vaccines and therapeutic applications for mammals, particularly humans, and for use as a plasmid backbone for a DNA vaccine or therapeutic DNA plasmid (e.g., encoding a therapeutic protein of interest) for delivery to a subject (e.g., human) of a protein of interest. The pCMV-Mkan vector is suitable for use in humans. It is small in size. For example, including the stuffer nucleotide sequence, the vector comprises 3741 nucleotide bases (SEQ ID NO:4). The vector includes a kanamycin resistant gene, instead of an ampicillin or tetracycline gene sequence, which may induce an undesirable allergic response or other undesirable side effect(s) in humans.

Within the polynucleotide sequence of a pCMV-Mkan DNA plasmid expression vector lacking the stuffer nucleotide sequence (as shown in SEQ ID NO:5), the human CMV promoter/enhancer/Intron A comprises nucleotide residues 3-1569; the BHG polyA sequence comprises nucleotide residues 1577-1816; the Kanamycin resistant gene sequence comprises nucleotide residues 2117-2932; and the ColE1 origin of replication comprises nucleotide residues 3040-3721. An exogenous coding sequence is cloned into the vector immediately following the CMV promoter polynucleotide sequence.

The polynucleotide sequence of SEQ ID NO:4 represents the polynucleotide sequence of the pCMV-Mkan DNA plasmid expression vector with the additional cloning site comprising at least EcoRI and KpnI recognition nucleotide sequences (“stuffer nucleotide sequence”). The stuffer nucleotide sequence serves as a cloning site and/or placeholder or marker of the position within the vector for insertion of a heterologous polypeptide-encoding nucleotide sequence. The stuffer nucleotide sequence, which comprises 26 nucleotide residues (atgcagtggaattcggtacctgatca, as shown in SEQ ID NO:13), is positioned in the polynucleotide sequence of SEQ ID NO:5 after the nucleotide residue at position 1571 of SEQ ID NO:5: An exogenous coding sequence is cloned into the polynucleotide sequence of SEQ ID NO:4 in place of the stuffer nucleotide sequence, which optionally may be removed (or not).

In one aspect, the invention provides an isolated, synthetic or recombinant nucleic acid comprising a polynucleotide sequence selected from: (a) a polynucleotide sequence that has at least about 60%, 70%, 80%, 85%, 90%, 95%, 96%, 97%, 98%, 99%, 99.5%, or 100% nucleic acid sequence identity to a full-length sequence of any polynucleotide sequence selected from the group of SEQ ID NOS:1-5, or a complementary polynucleotide sequence thereof; (b) a polynucleotide sequence comprising a polynucleotide fragment of any of SEQ ID NOS:1-5, wherein said polynucleotide fragment comprises at least about 1000 or at least about 2000 contiguous nucleotide bases of any of SEQ ID NOS:1-5, respectively; (c) a polynucleotide sequence that hybridizes under at least stringent conditions over substantially the entire length of polynucleotide sequence (a) or (b); and (d) a polynucleotide sequence of (a), (b), or (c) in which each thymidine residue in said polynucleotide sequence is replaced by a uracil residue. Some such nucleic acids of the invention comprise a vector, such as an expression vector. Some such nucleic acids comprise a DNA plasmid.

In another aspect, the invention provides an isolated, synthetic, or recombinant nucleic acid that comprises the polynucleotide sequence of any of SEQ ID NOS:1-5, or a complementary polynucleotide sequence thereof. In another aspect, the invention provides an isolated, synthetic or recombinant nucleic acid comprises the polynucleotide sequence of SEQ ID NOS:1-5, or a complementary polynucleotide sequence thereof, in which each thymidine residue is replaced by a uracil residue.

In another aspect, the invention provides an isolated, synthetic or recombinant nucleic acid comprising a polynucleotide sequence that has at least about 90% nucleic acid sequence identity to a polynucleotide sequence selected from the group of SEQ ID NOS:1, 2 and 5, or a complementary polynucleotide sequence thereof. In some instances, the polynucleotide sequence has at least about 95% nucleic acid sequence identity to a polynucleotide sequence selected from the group of SEQ ID NOS:1, 2, and 5, or a complementary polynucleotide sequence thereof. For some such nucleic acids, the polynucleotide sequence comprises a polynucleotide sequence selected from the group of SEQ ID NOS:1, 2, and 5, or a complementary polynucleotide sequence thereof. For some such nucleic acids, the polynucleotide sequence which hybridizes under at least stringent conditions over substantially the entire length of the polynucleotide sequence of SEQ ID NO:1, 2 or 5, or a complementary polynucleotide sequence thereof. The nucleic acid may be DNA or RNA.

Some such nucleic acids comprise a promoter and terminator signal sequence (such as a BGH polyadenylation sequence). The promoter may comprise a CMV promoter or a variant or mutant thereof. Alternatively, the promoter is a chimeric CMV promoter or a shuffled CMV promoter, including any shuffled promoter described in copending, commonly assigned International Patent Appn. WO 02/00879. Optionally, the nucleic acid further comprises an origin of replication, such as aColE1 origin of replication, and/or optionally further comprise a polynucleotide sequence encoding a kanamycin resistance marker. Optionally, the nucleic acid further comprises at least one polylinker and/or at least one restriction site for insertion of a polynucleotide sequence encoding a polypeptide.

In one aspect, nucleic acids of the invention comprise an expression vector capable of expressing at least one exogenous polypeptide upon incorporation into the expression vector of a polynucleotide encoding the at least one exogenous polypeptide. Typically, the at least one exogenous polynucleotide sequence is operably linked to a promoter polynucleotide sequence present in the nucleic acid. Depending upon the particular application and desired use, the nucleic acid further comprises at least one polynucleotide sequence encoding at least one antigen, co-stimulatory polypeptide, adjuvant, chemokine, or cytokine, or any combination thereof. Any antigen of interest may be employed, including, e.g., any wildtype antigen described in U.S. Pat. No. 6,541,011 or any chimeric or shuffled antigen produced by a method described U.S. Pat. No. 6,541,011, which is incorporated herein by reference in its entirety for all purposes.

In one aspect, the at least one antigen comprises at least one viral antigen, such as a flavivirus virus antigen or hepatitis A, B or C antigen or a variant or mutant. The antigen may be a wild-type antigen or a shuffled antigen. The antigen may induce an immune response against at least one serotype of a dengue virus selected from dengue-1, dengue-2, dengue-3, and dengue-4. A chimeric or shuffled dengue virus antigen, such as any such antigen described in copending, commonly assigned International patent application PCT Ser. No. 03/05,918, filed Feb. 26, 2003, may be included in a nucleic acid or vector of the invention.

In another aspect, the antigen comprises at least one cancer antigen, such as comprises against wild-type human epithelial cell adhesion molecule (EpCAM)/KSA or a mutant or variant thereof, including a shuffled or chimeric antigen that induces an immune response against EpCAM/KSA. The immune response induced by such antigens, as expressed from a vector of the invention, includes production of antibodies against human EpCAM and/or proliferation or activation of T cells.

In another aspect, the invention provides a nucleic acid vector that further comprises at least one polynucleotide sequence encoding at least one co-stimulatory polypeptide. Each polynucleotide sequence encoding at least one co-stimulatory polypeptide is operably linked to a promoter sequence present in the vector. In one aspect, the co-stimulatory polypeptide binds a mammalian CD28 receptor. The co-stimulatory polypeptide may comprise a wild-type B7-1 or B7-2 polypeptide or a variant or mutant thereof. Alternatively, the co-stimulatory polypeptide may comprise a shuffled B7-1 polypeptide that binds human CD28 and/or CTLA-4 receptor, including any shuffled or chimeric CD28BP or CTLA-4BP polypeptide described in copending, commonly assigned PCT application Ser. No. 01/19,973 (WO 02/00717). Exemplary monocistronic and bicistronic expression vectors, each encoding a CD28BP, are shown in FIGS. 3 and 4.

In another aspect, the invention provides an isolated, recombinant or synthetic nucleic acid vector comprising at least one nucleic acid of the invention described herein. Some nucleic acids of the invention comprise expression vectors that are capable of expressing at least one exogenous polypeptide. Such vectors may comprise a DNA plasmid vector. Exemplary vectors are shown in FIGS. 1-5. The expression vector comprises a promoter, and a terminator signal sequence, wherein the vector further comprises a heterologous nucleic acid coding sequence that encodes at least one polypeptide, the heterologous nucleic acid coding sequence operably linked to the promoter.

In one aspect, the invention provides a nucleic acid expression vector comprising a polynucleotide sequence having at least about 85, 90, 91, 92, 93, 94, 95, 96, 96, 98, 99, 99.5, or 100% sequence identity to a polynucleotide sequence selected from the group consisting of SEQ ID NOS:1-5 or to a complementary sequence thereof. Some such vectors comprise a polynucleotide sequence having at least about 85, 90, 95, 96, 96, 98, 99, 99.5, or 100% sequence identity to a polynucleotide sequence selected from the group consisting of SEQ ID NOS:1, 2, and 5 or a complementary sequence thereof.

A nucleic acid vector of the invention as described herein may further comprise at least a first heterologous or exogenous polynucleotide sequence encoding at least one antigen and at least a second heterologous or exogenous polynucleotide sequence encoding at least one co-stimulatory polypeptide. Each such polynucleotide sequence is operably linked to a promoter sequence present in the nucleic acid. The vector comprises two promoters; typically, the promoter is a promoter that directs synthesis of the heterologous polynucleotide sequence in a mammalian cells (e.g., CMV promoter or variant thereof). In one embodiment, the antigen is EpCAM or a variant thereof and the at least one co-stimulatory polypeptide binds human CD28 receptor. The co-stimulatory polypeptide may comprise a B7-1 variant, including any such variant described in commonly assigned PCT application Ser. No. 01/19,973 (WO 02/00717).

In another aspect, the invention provides an isolated, recombinant or synthetic nucleic acid vector comprising a polynucleotide sequence that hybridizes under at least stringent conditions over substantially the entire length of a polynucleotide sequence selected from the group of SEQ ID NOS:1-5, or a complementary polynucleotide sequence thereof.

Also provided is an isolated expression vector construct for the expression of a polypeptide in a mammalian cell, the expression vector comprising: (a) a first polynucleotide sequence having at least 85, 90, 91, 92, 93, 94, 95, 96, 97, 98, 99, 99.5, or 100% nucleic acid sequence identity to a polynucleotide sequence selected from the group of SEQ ID NOS:1, 2, and 5, wherein said first polynucleotide comprises a promoter for expression of the polypeptide in a mammalian cell and a terminator signal sequence; and (b) a second polynucleotide sequence encoding the polypeptide, wherein said second nucleic acid sequence is operably linked to the promoter.

Also provided is an isolated, synthetic or recombinant vector comprising the vector plasmid map shown in FIGS. 1, 2, 3, 4, or 5.

As noted above, a vector of the invention may comprise a bicistronic vector, comprising in addition to a polynucleotide sequence encoding at least a first polypeptide (e.g., antigen, marker, co-stimulatory molecule, adjuvant, chemokine, or cytokine (e.g., GM-CSF, IL-12, or IL-2)), a polynucleotide sequence encoding at least a second polypeptide (e.g., antigen, marker, co-stimulatory molecule, chemokine, or cytokine (e.g., GM-CSF, IL-12, or IL-2)). Such vector may also be tricistronic or of higher order, comprising at least one further (e.g., third, fourth, etc.) nucleotide acid sequence that encodes a polypeptide of interest. In one embodiment, the expression vector comprises a first polynucleotide coding sequence that encodes an antigen, such as a cancer antigen (such as, e.g., EpCAM (or mutant or variant polypeptide thereof)) or viral antigen. The second polynucleotide coding sequence may encode a co-stimulatory polypeptide, chemokine, or cytokine. Each polynucleotide coding sequence is operably linked to a promoter; the two promoters may be the same or different. The vector typically further comprises a terminator nucleotide sequence, such as a BGH polyA or SV40 polyA sequence.

Exemplary monocistronic expression vectors are shown in FIGS. 1, 2, and 5. An exemplary monocistronic expression vector encoding a polypeptide that binds human CD28 receptor is shown in FIG. 3. An exemplary expression bicistronic vector encoding a polypeptide that binds human CD28 receptor (CD28BP) and an EpCAM/KSA antigen is in FIG. 4. Alternatively, the two different polypeptide-encoding heterologous polynucleotide sequences can be substituted in the vector in FIG. 4 for the CD28BP and EpCAM/KSA-encoding polynucleotide sequences. The expression vector components shown in the vectors of FIGS. 1-5 may be used with any nucleic acid sequence inserted into the cloning site. Other expression vector elements that can be employed and other vector types and formats are described in detail below. An exemplary expression vector that includes a CD28BP polypeptide-encoding nucleotide sequence operably linked to a CMV promoter is shown in FIG. 3. An exemplary expression vector that comprises a CD28BP polypeptide-encoding nucleotide sequence operably linked to a first CMV promoter and an EpCAM/KSA antigen-encoding nucleotide sequence operably linked to a first CMV promoter is shown in FIG. 4.

In another aspect, the invention includes a DNA vaccine vector comprising at least one nucleic acid vector of the invention, wherein said nucleic acid vector further comprises at least one polynucleotide sequence encoding at least one antigen, antigenic polypeptide, or epitope of interest, wherein such at least one polynucleotide coding sequence is operably linked to a regulatory or promoter nucleotide sequence. The DNA vaccine vector may be a bicistronic vector that further comprises at least one polynucleotide sequence encoding an adjuvant, immunomodulator, cytokine, chemokine, or co-stimulator that enhances the immune response induced by the at least one antigen, antigenic polypeptide, or epitope. The antigen or antigenic polypeptide may, upon expression, form a virus-like particle (VLP).

In one aspect, some such nucleic acids or nucleic acid vectors of the invention further comprises at least one exogenous polynucleotide sequence encoding at least one antigen, co-stimulatory polypeptide, adjuvant, and/or cytokine, or any combination thereof. In some such aspects, the at least one exogenous polynucleotide sequence is operably linked to a promoter.

In some such aspects, the at least one antigen comprises at least one viral antigen, such as a flavivirus antigen. In a particular aspect, the at least one flavivirus antigen induces an immune response against at least serotype of a dengue virus selected from dengue-1, dengue-2, dengue-3, and dengue-4. A wild-type dengue virus envelope protein antigen or dengue virus antigen comprising a wild-type (wt) dengue virus premembrane (prM)/envelope protein or chimeric or shuffled dengue virus antigen. Exemplary nucleic acid sequences (including codon-optimized wt nucleotide sequences encoding four wt dengue virus envelope and four prM/envelope antigens) and protein sequences, including chimeric or shuffled dengue virus antigens that are capable of inducing an immune response against two or more dengue virus serotypes are set forth in Int'l patent application PCT Ser. No. 03/05,918, filed Feb. 26, 2003 incorporated herein by reference in its entirety for all purposes.

In another aspect, the at least one antigen comprises at least one cancer antigen, such as, e.g., epithelial cell adhesion molecule (EpCAM) (also known as KSA and EGP40) or a mutant or variant thereof. The cancer antigen may comprise an antigen that induces an immune response against human EpCAM. The cancer antigen may be a recombinant, shuffled, non-naturally occurring, or mutant antigen, or polypeptide variant of a known cancer antigen in which one or more amino acids of the known cancer antigen polypeptide sequence have been deleted and/or substituted with another amino acid, thereby resulting in a polypeptide variant or mutant of the known cancer antigen polypeptide, wherein the polypeptide variant or mutant induces an immune response (e.g., antibody or T cell response) against the known cancer antigen. For example, in one aspect of the invention, the cancer antigen induces production of antibodies against human EpCAM and/or a T cell activation or proliferation in a mammalian host.

In another aspect, a nucleic acid or vector of the invention further comprises at least one exogenous polynucleotide sequence encoding at least one co-stimulatory polypeptide. In one aspect, the at least one co-stimulatory polypeptide binds a mammalian CD28 receptor. In another aspect, the at least one co-stimulatory molecule binds a mammalian CTLA-4 receptor. In yet another aspect, the at least one co-stimulatory polypeptide comprises a B7-1 variant. The at least one polynucleotide sequence encoding the at least one co-stimulatory molecule is operably linked to a promoter, such as a CMV promoter (e.g., human or mammalian CMV promoter, Towne CMV promoter, AD169 CMV promoter, or the like) or a recombinant or shuffled promoter (e.g., variant of a CMV promoter).

In another aspect, the invention provides a nucleic acid comprising the polynucleotide sequence of each of SEQ ID NOS:1-5, or a complementary polynucleotide sequence thereof. Polynucleotide sequences that hybridize under at least stringent conditions over substantially the entire length of each such nucleic acid (e.g., any of SEQ ID NOS:1-5 or a complementary sequence thereof) are also included.

In another embodiment, the invention provides an expression vector comprising a polynucleotide sequence which comprises the nucleotide sequence of SEQ ID NO:5 in which the following additional 26-nucleotide residue segment is inserted after the nucleotide residue at position 1571 of SEQ ID NO:5: atgcagtggaattcggtacctgatca (SEQ ID NO:13). This stuffer nucleotide sequence includes EcoRI and KpnI recognition sites and, when included in the expression vector, is particularly useful as a cloning site in the vector for insertion of at least one heterologous gene(s) or protein-encoding polynucleotide(s) into the vector. The complete sequence of this expression vector, which includes the stuffer nucleotide sequence segment, is set forth in SEQ ID NO:4. However, the invention also includes a vector without the stuffer sequence.

If desired, the polynucleotide sequence of SEQ ID NO:4 can be modified by substituting one or more particular nucleic acid residues upstream of the initiator ATG (located at the 5′ end of the stuffer sequence) with a Kozak consensus sequence for initiation of translation in vertebrates as described in M. Kozak, Nucleic Acids Res. 15(20):8125-48 (1987). Alternatively, the polynucleotide sequence of SEQ ID NO:5 can be similarly modified at the nucleotide residue positions that correspond to those upstream of the initiator ATG in the polynucleotide sequence of SEQ ID NO:4 (that were substituted with a Kozak consensus sequence).

In another aspect, the invention provides an isolated or recombinant nucleic acid comprising a polynucleotide sequence that has at least about 85, 90, 91, 92, 93, 94, 95, 96, 97, 98, 99, 99.5 or 100% nucleic acid sequence identity to the polynucleotide sequence of SEQ ID NO:3 or 4, or a complementary polynucleotide sequence thereof. For some such isolated or recombinant nucleic acids, the polynucleotide sequence has at least about 90 or 95% nucleic acid sequence identity to the polynucleotide sequence of SEQ ID NO:3 or 4, or a complementary polynucleotide sequence thereof. For some such nucleic acids, the polynucleotide sequence comprises a polynucleotide sequence selected from the group of SEQ ID NOS:3 and 4, or a complementary polynucleotide sequence thereof. For some such nucleic acids, the isolated or recombinant nucleic acid comprises a polynucleotide sequence that hybridizes under at least stringent conditions over substantially the entire length of the polynucleotide sequence of SEQ ID NO:3 or 4, or a complementary polynucleotide sequence thereof. Some such nucleic acids comprise DNA or RNA. Some such nucleic acids comprise a promoter and terminator signal sequence, and optionally further comprise an origin of replication, such as, e.g., a ColE1 origin of replication. The terminator signal sequence may be a BGH polyadenylation sequence. The promoter may comprise a CMV promoter, such as human CMV (Towne strain), optionally with an enhancer and/or intron A, or a variant thereof. Alternatively, the promoter is a chimeric CMV promoter. Some such nucleic acids further comprise a polynucleotide sequence encoding a kanamycin resistance marker.

Some such isolated or recombinant nucleic acids further comprise at least one polylinker to permit insertion of a heterologous gene. Some such nucleic acids further comprise at least one restriction site for insertion of a polynucleotide sequence encoding a polypeptide. In some instances, the nucleic acid is an expression vector capable of expressing at least one exogenous polypeptide upon incorporation into the expression vector of a polynucleotide encoding the at least one exogenous polypeptide. The at least one exogenous polynucleotide sequence is typically operably linked to a promoter polynucleotide sequence present in the nucleic acid. In some embodiments, the isolated or recombinant nucleic acid further comprises at least one polynucleotide sequence encoding at least one antigen, co-stimulatory polypeptide, adjuvant, chemokine, or cytokine, or any combination thereof. In one embodiment, the at least one antigen comprises at least one viral antigen, such as a flavivirus virus antigen or hepatitis antigen (e.g., hepatitis surface antigen or envelope protein).

Any polypeptide described herein may further include a secretion signal or localization signal sequence, e.g., a signal sequence, an organelle targeting sequence, a membrane localization sequence, and the like. Any polypeptide described herein may further include a sequence that facilitates purification, e.g., an epitope tag (such as, e.g., a FLAG epitope), a polyhistidine tag, a GST fusion, and the like. The polypeptide optionally includes a methionine at the N-terminus. Any polypeptide described herein optionally includes one or more modified amino acids, such as a glycosylated amino acid, a PEG-ylated amino acid, a farnesylated amino acid, an acetylated amino acid, a biotinylated amino acid, a carboxylated amino acid, a phosphorylated amino acid, an acylated amino acid, or the like. Any polypeptide described herein further may be incorporated into a fusion protein, e.g., a fusion with an immunoglobulin (Ig) sequence. Accordingly, the nucleic acids and vectors of the invention may further include any nucleotide sequence(s) encoding any such polypeptide sequence, e.g., secretion signal or signal sequence, purification sequence, tag, or fusion protein.

The invention also includes RNA nucleotide sequences that correspond to each of the DNA nucleotide sequences (including expression vector sequences) of the invention. For example, included is an RNA nucleotide sequence comprising the DNA nucleotide sequence any of SEQ ID NOS:1-5 or the complementary sequence thereof, wherein a uracil residue is substituted for each thymidine residue in said DNA sequence, and a complementary sequence of each such RNA sequence. The invention further provides a virus or viral vector comprising a nucleic acid or polynucleotide (RNA or DNA) of the invention.

Making Nucleic Acids

Nucleic acids, polynucleotides, oligonucleotides, nucleic acid fragments, and vectors of the invention can be prepared by standard solid-phase methods, according to known synthetic methods. Typically, fragments of up to about 100 bases are individually synthesized, then joined (e.g., by enzymatic or chemical ligation methods, or polymerase mediated recombination methods) to form essentially any desired continuous sequence. For example, the polynucleotides and oligonucleotides of the invention can be prepared by chemical synthesis using, e.g., classical phosphoramidite method described by, e.g., Beaucage et al. (1981) Tetrahedron Letters 22:1859-69, or the method described by Matthes et al. (1984) EMBO J 3:801-05, e.g., as is typically practiced in automated synthetic methods. According to the phosphoramidite method, oligonucleotides are synthesized, e.g., in an automatic DNA synthesizer, purified, annealed, ligated and cloned into appropriate vectors.

In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, e.g., PeptidoGenic (pkim@ccnet.com), HTI Bio-products, Inc. (http://www.htibio.com), BMA Biomedicals Ltd. (U.K.), Bio.Synthesis, Inc., and many others.

Certain polynucleotides of the invention may also be obtained by screening cDNA libraries (e.g., libraries generated by recombining nucleic acids, such as homologous nucleic acids,, as in typical recursive sequence recombination methods) using oligonucleotide probes that can hybridize to or PCR-amplify polynucleotides, which encode polypeptides of the invention and/or fragments of those polypeptides. Procedures for screening and isolating cDNA clones are well known to those of skill in the art. Such techniques are described in, e.g., Berger and Kimmel, Guide to Molecular Cloning Techniques, Methods in Enzymol. Vol. 152, Acad. Press, Inc., San Diego, Calif. (“Berger”); Sambrook, Goeddel, and Ausubel, all supra. Some polynucleotides of the invention can be obtained by altering a naturally occurring backbone, e.g., by mutagenesis, recursive sequence recombination (e.g., shuffling), or oligonucleotide recombination. In other cases, such polynucleotides can be made in silico or through oligonucleotide recombination methods as described in the references cited herein.

As described in more detail herein, the nucleic acids of the invention include polynucleotide sequences that encode polypeptide sequences and fragments thereof, polynucleotide sequences complementary to these polynucleotide sequences and fragments thereof, polynucleotides that hybridize under at least stringent conditions to nucleotide sequences defined herein, novel fragments of coding sequences and complementary sequences thereof, and variants, analogs, and homologue derivatives of all of the above. A nucleotide coding sequence may encodes a particular polypeptide or domain, region, or fragment of the polypeptide. A coding sequence may code for a polypeptide or fragment thereof having a functional property, such as a an ability to bind a receptor, induce or suppress T cell proliferation in conjunction with stimulation of T cell receptor (by, e.g., an antigen or anti-CD3 antibodies (Ab), or induce or stimulate a cytokine response as described herein. The polynucleotides of the invention can be in the form of RNA or in the form of DNA, and include mRNA, cRNA, synthetic RNA and DNA, and cDNA. The polynucleotides can be double-stranded or single-stranded, and if single-stranded, can be the coding strand or the non-coding (anti-sense, complementary) strand. The polynucleotides optionally include the coding sequence of a polypeptide (i) in isolation, (ii) in combination with one or more additional coding sequences, so as to encode, e.g., a fusion protein, a pre-protein, a prepro-protein, or the like, (iii) in combination with non-coding sequences, such as introns, control elements, such as a promoter (e.g., naturally occurring or recombinant or shuffled promoter), a terminator element, or 5′ and/or 3′ untranslated regions effective for expression of the coding sequence in a suitable host, and/or (iv) in a vector, cell, or host environment in which coding sequence is a heterologous gene. Polynucleotide sequences can also be found in combination with typical compositional formulations of nucleic acids, including in the presence of carriers, buffers, adjuvants, excipients, and the like, as are known to those of ordinary skill in the art. Nucleotide fragments typically comprise at least about 500 nucleotide bases, usually at least about 600, 650, or 700 bases, and often 750 or more bases. The nucleotide fragments, variants, analogs, and homologue derivatives of polynucleotides of the invention may have hybridize under highly stringent conditions to a polynucleotide or homologue sequence described herein and/or encode amino acid sequences having at least one of the properties of receptor binding, ability to alter an immune response via, e.g., T cell activation /proliferation, and cytokine production of polypeptides described herein.

Using Nucleic Acids and Vectors

The nucleic acids, vectors, and fragments, variants, and homologues thereof of the invention have a variety of uses in, for example, recombinant production or expression of one or more polypeptides. For example, a nucleic acid of the invention typically serves as an expression vector or component or fragment thereof for expression of a polypeptide whose polynucleotide sequence has been incorporated into a cloning site of the vector. Nucleic acids, vectors, and fragments, variants, and homologues thereof of the invention comprising exogenous polynucleotide sequences which encode one or more exogenous polypeptides or proteins, fragments, variants or homologues thereof, related fusion polypeptides or proteins, or functional equivalents thereof (i.e., components), direct the expression of such components in appropriate host cells.

Such nucleic acids, vectors, and fragments, variants, and homologues thereof are also useful in methods of the invention, including therapeutic methods for inducing or enhancing an immune response in a subject to whom the nucleic acid, vector or fragment, variant, and homologue thereof of the invention is administered, and methods of treating disorders and diseases in subjects, including mammals, as described in more detail below. In particular, such nucleic acids, vectors and fragments, variants, and homologues thereof are useful in DNA vaccine applications, gene therapy application and therapeutic or prophylactic applications wherein in vivo or ex vivo delivery of a protein of interest is desired. Such vectors, nucleic acids and fragments, variants, and homologues thereof of the invention can be administered to a subject by any one of the delivery routes described below (including, but not limited to, e.g., intramuscularly, intradermally, subdermally, subcutaneously, orally, intraperitoneally, intrathecally, intravenously, mucosally, systemically, parenterally, via inhalation, or placed within a cavity of the body (including, e.g., during surgery)). Vectors encoding exogenous polypeptides are optionally administered to a cell, tissue or subject to accomplish a therapeutically or prophylactically useful process or to express or introduce therapeutically and/or prophylactically useful polypeptides.

Modified Coding Sequences

As will be understood by those of ordinary skill in the art, it can be advantageous to modify a coding nucleotide sequence to enhance its expression in a particular host. The genetic code is redundant with 64 possible codons, but most organisms preferentially use a subset of these codons. The codons that are utilized most often in a species are called optimal codons, and those not utilized very often are classified as rare or low-usage codons (see, e.g., Zhang, S. P. et al. (1991) Gene 105:61-72). Codons can be substituted to reflect the preferred codon usage of the host, a process called “codon optimization” or “controlling for species codon bias.” Optimized coding sequence containing codons preferred by a particular prokaryotic or eukaryotic host (see, e.g., Murray, E. et al. (1989) Nuc Acids Res 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (Dalphin, M. E. et al. (1996) Nuc Acids Res 24:216-218).

Nucleic acids and polynucleotide sequences of the present invention can be engineered in order to alter a coding sequence described herein for a variety of reasons, including but not limited to, alterations which modify the cloning, processing and/or expression of the gene product. For example, alterations may be introduced using techniques which are well known in the art, e.g., site-directed mutagenesis, to insert new restriction sites, to alter glycosylation and/or pegylation patterns, to change codon preference, to introduce splice sites, etc. Further details regarding silent and conservative substitutions are provided below.

The present invention includes recombinant or synthetic nucleic acid constructs comprising one or more of the nucleic acid sequences as broadly described above. The constructs may comprise a vector, such as, a plasmid, a cosmid, cloning vector, expression vector, a virus, a virus-like particle, or the like, into which a nucleic acid sequence (e.g., one which encodes a polypeptide or fragment thereof of interest) has been inserted, in a forward or reverse orientation. In one aspect, the construct further comprises regulatory sequences, including, for example, a promoter, operably linked to the nucleic acid sequence, and optionally a termination sequence. Large numbers of suitable promoters, regulatory sequences, and termination sequences are known to those of skill in the art, and are commercially available and can be substituted for a respective sequence in one of the vectors of the invention.

General texts that describe molecular biological techniques useful herein, including the use of vectors, promoters and many other relevant topics, include Berger, Sambrook (2001), Goeddel, and Ausubel, all supra. Examples of techniques sufficient to direct persons of skill through in vitro amplification methods, including the polymerase chain reaction (PCR) the ligase chain reaction (LCR), Q∃-replicase amplification and other RNA polymerase mediated techniques (e.g., NASBA), e.g., for the production of the homologous nucleic acids of the invention are found in Berger, Sambrook, Goeddel, and Ausubel, all supra, as well as Mullis et al. (1987) U.S. Pat. No. 4,683,202; PCR Protocols: A Guide to Methods and Applications (Innis et al., eds.) Academic Press Inc. San Diego, Calif. (1990) (“Innis”); Arnheim & Levinson (Oct. 1, 1990) C&EN 36-47; The Journal Of NIH Research (1991) 3:81-94; (Kwoh et al. (1989) Proc Natl Acad Sci USA 86:1173-1177; Guatelli et al. (1990) Proc Natl Acad Sci USA 87:1874-1878; Lomeli et al. (1989) J Clin Chem 35:1826-1831; Landegren et al. (1988) Science 241:1077-1080; Van Brunt (1990) Biotechnology 8:291-294; Wu and Wallace (1989) Gene 4:560-569; Barringer et al. (1990) Gene 89:117-122, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kilobases (kb) are generated. One of skill will appreciate that essentially any RNA can be converted into a double stranded DNA suitable for restriction digestion, PCR expansion and sequencing using reverse transcriptase and a polymerase. See Ausubel, Sambrook, Goeddel, METH. IN ENZYMOL., and Berger, all supra.

Host Cells and Regulatory Sequences

The invention also provides host cells comprising any of the vectors or nucleic acids described herein. In one aspect, the invention provides a cell or population of cells comprising at least one nucleic acid or nucleic acid vector of the invention described herein. In one embodiment, the cell expresses a polypeptide encoded by the nucleic acids herein. The invention includes a host cell comprising at least one nucleic acid comprising at least one polynucleotide sequence having at least about 90, 91, 92, 93, 94, 95, 96, 97, 98, 99, 99.5% or more sequence identity to a polynucleotide sequence selected from the group consisting of SEQ ID NOS:1-5. The host cell typically comprises a eukaryotic cell. Cells and transgenic animals that include any polypeptide or nucleic acid herein, e.g., produced by transduction of the vector, are also a feature of the invention. Also included is a mammalian cell transformed or transfected with at least one nucleic acid or vector of the invention. The invention also provides compositions comprising at least one host cell comprising at least one nucleic acid or vector of the invention and an excipient. Preferably, the composition is a pharmaceutical composition and the excipient is pharmaceutically acceptable excipient carrier.

The present invention also provides host cells that are transduced with vectors of the invention, and the production of polypeptides of the invention by recombinant techniques. Host cells are genetically engineered (e.g., transduced, transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying a polynucleotide or gene on interest. The culture conditions, such as temperature, pH, and the like, are those previously used with the host cell selected for expression, and will be apparent to those skilled in the art and in the references cited- herein, including, e.g., Freshney (1994) Culture of Animal Cells, a Manual of Basic Technique, third edition, Wiley-Liss, New York and the references cited therein.

Polypeptides of interest can also be produced in non-animal cells such as plants, yeast, fungi, bacteria and the like. In addition to Sambrook, Goeddel, Berger and Ausubel, details regarding cell culture are found in, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.); Atlas & Parks (eds.) The Handbook of Microbiological Media (1993) CRC Press, Boca Raton, Fla.

The nucleic acid sequence in the expression vector is operatively linked to an appropriate transcription control sequence (promoter) to direct mRNA synthesis. Examples of such promoters include: LTR or SV40 promoter, CMV promoter, E. coli lac or trp promoter, phage lambda P_{L} promoter and other promoters known to control expression of genes in eukaryotic cells or prokaryotic cells or their viruses. The expression vector also contains a ribosome binding site for translation initiation, and a transcription terminator. The vector optionally includes appropriate sequences for amplifying expression, e.g., an enhancer. In addition, the expression vectors optionally comprise one or more selectable marker genes to provide a phenotypic trait for selection of transformed host cells, such as dihydrofolate reductase, hygromycin, blasticidine or neomycin resistance for eukaryotic cell culture, or such as kanamycin, tetracycline or ampicillin resistance in E. coli.

The vector containing the appropriate DNA sequence encoding an exogenous polypeptide, as well as an appropriate promoter or control sequence, may be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 or Bowes melanoma; plant cells, etc. It is understood that not all cells or cell lines need to be capable of producing fully functional polypeptides of interest or fragments thereof (e.g., exogenous therapeutic or prophylactic polypeptide(s) whose corresponding nucleic acid sequence(s) has been incorporated into a nucleic acid vector of the invention); for example, fragments of a polypeptide of interest may be produced in a bacterial or other expression system. The invention is not limited by the host cells employed.

In bacterial systems, the expression vector can be designed depending upon the use intended for the incorporated exogenous nucleic acid or the encoded exogenous polypeptide or fragment thereof. For example, when large quantities of an exogenous polypeptide or fragment thereof is needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which exogenous nucleotide coding sequence may be ligated into the vector in-frame with sequences for the amino-terminal Met and the subsequent 7 residues of beta-galactosidase so that a hybrid protein is produced; pIN vectors (Van Heeke & Schuster (1989) J Biol Chem 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH may be used for production of the polypeptides of interest. For reviews, see Ausubel, supra, Berger, supra, and Grant et al. (1987) Methods in Enzymology 153:516-544.

In mammalian host-cells, expression vectors and expression systems of the invention may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome results in a viable virus capable of expressing polypeptide of interest in infected host cells (Logan and Shenk (1984) Proc Natl Acad Sci USA 81:3655-3659). In addition, transcription enhancers, such as the Rous sarcoma virus (RSV) enhancer, are used to increase expression in mammalian host cells. Host cells, media, expression systems, and methods of production include those known for cloning and expression of one or more of a variety of antigens and/or mammalian B7-1 polypeptides or variants thereof.

Promoters for use in vectors and nucleic acids of the invention and with exogenous polynucleotide sequences of interest include recombinant, mutated, or recursively recombined (e.g., shuffled) promoters, including optimized recombinant CMV promoters, as described in copending, commonly assigned PCT application Ser. No. 01/20,123, entitled “Novel Chimeric Promoters,” filed Jun. 21, 2001, which published with International Publication No. WO 02/00897, incorporated herein by reference in its entirety for all purposes. In some embodiments, a recombinant or shuffled promoter having an optimized expression for a particular use with an exogenous polynucleotide incorporated into the vector. For example, in some therapeutic and/or prophylactic methods or applications, where a lower level expression of an exogenous polypeptide (e.g., antigen, co-stimulatory molecule, etc.) is desired (than is typically obtained with a CMV promoter, such as a WT human CMV promoter), at least one recombinant or chimeric CMV promoter nucleotide sequence that is optimized to provide for reduced or suppressed expression levels of the exogenous polypeptide is used.

Such promoter(s) is operably linked in the expression vector to either or both the exogenous polynucleotide and/or one or more associated antigens (e.g., any antigen, e.g., viral antigen (e.g., flavivirus antigen, such as a dengue antigen, malaria antigen, hepatitis A,B,C antigen, or HIV antigen, etc. or a chimeric, shuffled, mutant, or variant antigen thereof), cancer antigen (e.g., EpCAM/KSA or chimeric, shuffled, mutant, variant of EpCAM/KSA), etc. In other embodiments, one or more recombinant, mutant, or chimeric CMV promoters optimized for the particular application can be used, where differential expression between a first exogenous polypeptide of interest and at least one additional exogenous polypeptide of interest in one or more vectors of the invention is desired (e.g., where it is desirable to express varying amounts of various exogenous polypeptides, since their respective concentrations influence or affect one another, and/or where it is desirable to express a comparably higher level of at least one exogenous polypeptide (e.g., antigen) for effective treatment). For example, in some applications, a low expression level of an exogenous polypeptide (e.g., co-stimulatory polypeptide) and a relatively higher expression level of antigen (e.g., cancer antigen) is desired, since it may be particularly useful for successful therapeutic or prophylactic treatment of a particular condition or disease (e.g., cancer, such as colon, colorectal, or rectal cancer).

Specific initiation signals in the vector can aid in efficient translation of an exogenous polynucleotide coding sequence and/or fragments thereof. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous nucleic acid transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (see, e.g., Scharf D. et al. (1994) Results Probl Cell Differ 20:125-62; and Bittner et al. (1987) Methods in Enzymol 153:516-544).

In a nucleic acid or vector of the invention, a polynucleotides encoding an exogenous polypeptide or fragment thereof can also be fused, for example, in-frame to nucleic acid encoding a secretion/localization sequence, to target polypeptide expression to a desired cellular compartment, membrane, or organelle, or to direct polypeptide secretion to the periplasmic space or into the cell culture media. Such sequences are known to those of skill, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

In a further embodiment, the present invention relates to host cells containing any of the above-described nucleic acids, vectors, or other constructs of the invention. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, gene or vaccine gun, injection, or other common techniques (see, e.g., Davis, L., Dibner, M., and Battey, I. (1986) Basic Methods in Molecular Biology) for in vivo, ex vivo, or in vitro methods.

A host cell strain is optionally chosen for its ability to modulate the expression of the inserted sequences or to process the expressed protein in the desired fashion. Such modifications of the protein include, but are not limited to, acetylation, carboxylation, pegylation, glycosylation, phosphorylation, lipidation and acylation. Post-translational processing, which cleaves a “pre” or a “prepro” form of the protein, may also be important for correct insertion, folding and/or function. Different host cells such as E. coli, Bacillus sp., yeast or mammalian cells such as CHO, HeLa, BHK, MDCK, HEK 293, W138, etc. have specific cellular machinery and characteristic mechanisms for such post-translational activities and may be chosen to ensure the correct modification and processing of the introduced foreign protein.

For long-term, high-yield production of recombinant proteins, stable expression can be used. For example, cell lines that stably express a polypeptide of the invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells can be proliferated using tissue culture techniques appropriate to the cell type.

Host cells transformed with a vector of the invention comprising a nucleotide sequence encoding an exogenous polypeptide or fragment thereof are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding exogenous polypeptides can be designed with signal sequences, which direct secretion of the mature polypeptides through a prokaryotic or eukaryotic cell membrane.

The vector of the present invention comprising an exogenous polypeptide-encoding polynucleotide optionally comprises a coding sequence or fragment thereof fused in-frame to a marker sequence that, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I. et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the exogenous sequence is useful to facilitate purification.

For example, one expression vector possible to use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281) while the enterokinase cleavage site provides a method for separating the exogenous polypeptide of interest from the fusion protein. pGEX vectors (Promega; Madison, Wis.) are optionally used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Polypeptide Production and Recovery

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Eukaryotic or microbial cells employed in expression of the exogenous proteins expressed by vectors of the invention can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian) and archebacterial origin. See, e.g., Sambrook, Ausubel, Goeddel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential, Techniques John Wiley and Sons, NY; Humason (1979) Animal Tissue Techniques, fourth edition W.H. Freeman and Company; and Ricciardelli et al. (1989) In vitro Cell Dev Biol 25:1016-1024. For plant cell culture and regeneration see, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.) and Plant Molecular Biology (1993) R. R. D. Croy (ed.) Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (1997) also from Sigma-Aldrich, Inc (St Louis, Mo.) (“Sigma-PCCS”).

Exogenous polypeptides expressed from nucleic acids and vectors of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the exogenous protein or fragments thereof. Finally, high performance liquid chromatography (HPLC) can be employed in the final purification steps. In addition to the references noted, supra, a variety of purification methods are well known in the art, including, e.g., those set forth in Sandana (1997) Bioseparation of Proteins, Academic Press, Inc.; Bollag et al. (1996) Protein Methods, 2nd Edition Wiley-Liss, N.Y.; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, N.Y.; Janson and Ryden (1998) Protein Purification: Principles, High Resolution Methods and Applications, Second Edition Wiley-VCH, NY; and Walker (1998) Protein Protocols on CD-ROM Humana Press, NJ.

In vitro Expression Systems

Cell-free transcription/translation systems can also be employed to produce exogenous polypeptides or fragments thereof using DNAs or RNAs of the present invention or fragments thereof. Several such systems are commercially available. A general guide to in vitro transcription and translation protocols is found in Tymms (1995) In vitro Transcription and Translation Protocols: Methods in Molecular Biology Volume 37, Garland Publishing, NY.

In vivo Polypeptide Expression

Vectors of the invention comprising one or more exogenous polynucleotide sequences encoding one or more exogenous therapeutic polypeptides (each polynucleotide sequence cloned into a cloning site(s) of a vector using standard techniques) are particularly useful for in vivo therapeutic applications, using techniques well known to those skilled in the art. For example, cultured cells are engineered ex vivo with at least one exogenous polynucleotide (DNA or RNA) and/or other polynucleotide sequences encoding, e.g., at least one of an antigen, cytokine, other co-stimulatory molecule, adjuvant, etc., and the like, with the engineered cells then being returned to the patient. Cells may also be engineered in vivo for expression of one or more polypeptides in vivo.

Gene therapy and genetic vaccines provide methods for combating chronic infectious diseases (e.g., HIV infection, viral hepatitis), or preventing infectious disease (e.g., viral infection (dengue, malaria, HIV infection, hepatitis A, B, C, etc.) or bacterial infection, as well as non-infectious diseases, including cancer, allergies, autoimmune disorders and some forms of congenital defects such as enzyme deficiencies, and such methods can be employed with vectors of the invention, wherein such vectors include exogenous polynucleotide sequence(s) encoding a polypeptide useful in treating or preventing such disease or in enhancing the immune response of the subject. Several approaches for introducing nucleic acids and vectors into cells in vivo, ex vivo and in vitro have been used and can be employed with vectors of the invention. These approaches include liposome based gene delivery (Debs and Zhu (1993) WO 93/24640 and U.S. Pat. No. 5,641,662; Mannino and Gould-Fogerite (1988) BioTechniques 6(7):682-691; Rose, U.S. Pat. No. 5,279,833; Brigham (1991) WO 91/06309; and Felgner et al. (1987) Proc Natl Acad Sci USA 84:7413-7414; Brigham et al. (1989) Am J Med Sci 298:278-281; Nabel et al. (1990) Science 249:1285-1288; Hazinski et al. (1991) Am J Resp Cell Molec Biol 4:206-209; and Wang and Huang (1987) Proc Natl Acad Sci USA 84:7851-7855); adenoviral vector mediated gene delivery, e.g., to treat cancer (see, e.g., Chen et al. (1994) Proc Natl Acad Sci USA 91:3054-3057; Tong et al. (1996) Gynecol Oncol 61:175-179; Clayman et al. (1995) Cancer Res. 5:1-6; O'Malley et al. (1995) Cancer Res 55:1080-1085; Hwang et al. (1995) Am J Respir Cell Mol Biol 13:7-16; Haddada et al. (1995) Curr Top Microbiol Immunol. 1995 (Pt. 3):297-306; Addison et al. (1995) Proc Natl Acad Sci USA 92:8522-8526; Colak et al. (1995) Brain Res 691:76-82; Crystal (1995) Science 270:404-410; Elshami et al. (1996) Human Gene Ther 7:141-148; Vincent et al. (1996) J Neurosurg 85:648-654), and many others. Replication-defective retroviral vectors harboring therapeutic polynucleotide sequence as part of the retroviral genome have also been used, particularly with regard to simple MuLV vectors. See, e.g., Miller et al. (1990) Mol Cell Biol 10:4239 (1990); Kolberg (1992) J NIH Res 4:43, and Cornetta et al. (1991) Hum Gene Ther 2:215). Nucleic acid transport coupled to ligand-specific, cation-based transport systems (Wu and Wu (1988) J Biol Chem, 263:14621-14624) has also been used. Naked DNA expression vectors have also been described (Nabel et al. (1990), supra); Wolff et al. (1990) Science, 247:1465-1468). In general, these approaches can be adapted to the invention by incorporating nucleic acids of interest into the appropriate vector(s) described herein. Additional approaches are discussed below.

General texts, which describe gene therapy protocols, which can be adapted to the present invention by introducing the nucleic acids of the invention into patients, include, e.g., Robbins (1996) Gene Therapy Protocols, Humana Press, NJ, and Joyner (1993) Gene Targeting: A Practical Approach,

IRL Press, Oxford, England.

Sequence Variations

Silent Variations

Because of the degeneracy of the genetic code, a large number of functionally identical nucleic acids encode any given polypeptide. For instance, inspection of the codon table (Table 1) shows that codons AGA, AGG, CGA, CGC, CGG, and CGU all encode the amino acid arginine. Thus, at every position in a nucleic acid sequence where an arginine is specified by a codon, the codon can be altered to any of the corresponding codons described above without altering the encoded polypeptide. Such nucleic acid variations are “silent variations” are one species of “conservatively modified variations.” It is understood that U in an RNA sequence corresponds to T in a DNA sequence.

TABLE 1
Codon Table

| | |
|---|---|
| Amino acids | Codon |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will thus be appreciated by those skilled in the art that due to the degeneracy of the genetic code, a multitude of nucleic acid sequences encoding a polypeptide may be produced, some of which may bear minimal sequence homology to the nucleic acid sequences explicitly disclosed herein. One of ordinary skill in the art will recognize that each codon in a nucleic acid (except AUG and UGC, which are ordinarily the only codon for methionine and tryptophan, respectively) can be modified by standard techniques to encode a functionally identical polypeptide. Accordingly, each silent variation of a nucleic acid that encodes a polypeptide is implicit in any described sequence. The invention also provides each and every possible variation of a nucleic acid sequence encoding a polypeptide that can be made by selecting combinations based on possible codon choices. These combinations are made in accordance with the standard triplet genetic code (codon) (e.g., as set forth in Table 1), as applied to the nucleic acid sequence encoding a polypeptide of the invention or fragment thereof. All such variations of every nucleic acid herein are specifically provided and described by consideration of the sequence in combination with the genetic code. One of skill is fully able to generate any silent substitution of the sequences listed herein. For example, the invention includes polynucleotides comprising one or more silent variations of any polynucleotide sequence selected from SEQ ID NO:1 or the complementary polynucleotide sequence thereof. Also included are polynucleotides comprising one or more silent variations of a nucleotide segment or fragment of SEQ ID NO:1, or complementary polynucleotide sequence thereof. Also provided are polypeptides encoded by all such polynucleotides of the invention comprising one or more silent variations.

Conservative Variations

“Conservatively modified variations,” or simply “conservative variations,” of a particular nucleic acid sequence refer to those nucleic acid sequences that encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence of the invention are “conservatively modified variations” where the alterations result in the deletion, addition, and/or substitution of an amino acid with a chemically similar amino acid.

Conservative substitution tables providing functionally similar amino acids are well known in the art. Table 2 sets forth six exemplary groups that contain amino acids that are “conservative substitutions” for one another.

TABLE 2
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Additional groups of amino acids can also be formulated. For example, amino acids can be grouped by similar function or chemical structure or composition (e.g., acidic, basic, aliphatic, aromatic, sulfur-containing). For example, an aliphatic grouping may comprise: Glycine (G), Alanine, Valine, Leucine, and Isoleucine. Other groups containing amino acids that are conservative substitutions for one another include: Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also Creighton (1984) Proteins, W.H. Freeman and Company, for additional groupings of amino acids.

Thus, “conservatively substituted variations” of a polypeptide sequence of the present invention include substitutions of a small percentage, typically less than 5%, more typically less than 4%, 3%, 2%, or 1%, of the amino acids of the sequence, with a conservatively selected amino acid of the same conservative substitution group.

For example, a conservatively substituted variation of the polyp eptide identified herein may contain “conservative substitutions,” according to the six groups defined above, in up to 15 amino acid residues (i.e., 5% of the amino acids) in the polypeptide. Listing of a polypeptide or protein sequence herein, in conjunction with the above substitution table, provides an express listing of all conservatively substituted polypeptide or protein sequences.

The addition of one or more nucleic acids or sequences that do not alter the encoded activity of a nucleic acid molecule of the invention, such as the addition of a non-functional sequence, is a conservative variation of the basic nucleic acid molecule, and the addition of one or more amino acid residues that do not alter the activity of a polypeptide of the invention is a conservative variation of the basic polypeptide. Both such types of additions are features of the invention.

One of skill will appreciate that many conservative variations of the nucleic acid sequence constructs that are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence that encodes an-amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention. The invention includes polynucleotides of the invention comprising one or more such conservative variations.

Nucleic Acid Hybridization

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, N.Y.) (hereinafter “Tjissen”), as well as in Ausubel, supra, Hames and Higgins (1995) Gene Probes 1, IRL Press at Oxford University Press, Oxford, England (Hames and Higgins 1) and Hames and Higgins (1995) Gene Probes 2, IRL Press at Oxford University Press, Oxford, England (Hames and Higgins 2) provide details on the synthesis, labeling, detection and quantification of DNA and RNA, including oligonucleotides.

An indication that two nucleic acid sequences are substantially identical is that the two molecules hybridize to each other under at least stringent conditions. The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization wash conditions” and “stringent hybridization conditions” in the context of nucleic acid hybridization experiments, such as Southern and northern hybridizations, are sequence dependent, and are different under different environmental parameters. An extensive guide to hybridization of nucleic acids is found in Tijssen (1993), supra, and in Hames and Higgins 1 and Hames and Higgins 2, supra.

For purposes of the present invention, generally, “highly stringent” hybridization and wash conditions are selected to be about 5° C. (or less) lower than the thermal melting point (Tₘ) for the specific sequence at a defined ionic strength and pH (as noted below, highly stringent conditions can also be referred to in comparative terms). The Tₘ is the temperature (under defined ionic strength and pH) at which 50% of the test sequence hybridizes to a perfectly matched probe. In other words, the Tₘ indicates the temperature at which the nucleic acid duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides. Typically, under “stringent conditions,” a probe will hybridize to its target subsequence, but to no other sequences. “Very stringent conditions” are selected to be equal to the Tₘ for a particular probe.

After hybridization, unhybridized nucleic acid material can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lower the background signal, typically with only the specific signal remaining. See, Rapley, R. and Walker, J. M. eds., Molecular Biomethods Handbook (Humana Press, Inc. 1998) (hereinafter “Rapley and Walker”), which is incorporated herein by reference in its entirety for all purposes.

The Tₘ of a DNA-DNA duplex can be estimated using equation (1):

Tₘ(° C.)=81.5° C.+16.6 (log₁₀M)+0.41(% G+C)−0.72(% f)−500/n,

where M is the molarity of the monovalent cations (usually Na+), (% G+C) is the percentage of guanosine (G) and cystosine (C ) nucleotides, (% f) is the percentage of formalize and n is the number of nucleotide bases (i.e., length) of the hybrid. See, Rapley and Walker, supra.

The Tₘ of an RNA-DNA duplex can be estimated using equation (2):

Tₘ(° C.)=79.8° C.+18.5 (log₁₀M)+0.58 (% G+C)−11.8(% G+C)²−0.56(% f)−820/n,

where M is the molarity of the monovalent cations (usually Na+), (% G+C)is the percentage of guanosine (G ) and cystosine (C ) nucleotides, (% f) is the percentage of formamide and n is the number of nucleotide bases (i.e., length) of the hybrid. Id. Equations 1 and 2 above are typically accurate only for hybrid duplexes longer than about 100-200 nucleotides. Id.

The Tm of nucleic acid sequences shorter than 50 nucleotides can be calculated as follows:

Tₘ(° C.)=4(G+C)+2(A+T), where A (adenine), C, T (thymine), and G are the numbers of the corresponding nucleotides.

An example of stringent hybridization conditions for hybridization of complementary nucleic acids which have more than 100 complementary residues on a filter in a Southern or Northern blot is 50% formalin (or formamide) with 1 mg of heparin at 42° C., with the hybridization being carried out overnight. An example of stringent wash conditions is a 0.2×SSC wash at 65° C. for 15 minutes (see Sambrook, supra, for a description of SSC buffer). Often, the high stringency wash is preceded by a low stringency wash to remove background probe signal. An example low stringency wash is 2×SSC at 40° C. for 15 minutes. An example of highly stringent wash conditions is 0.15M NaCl at 72° C. for about 15 minutes. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na⁺ ion, typically about 0.01 to 1.0 M Na⁺ ion concentration (or other salts) at pH 7.0 to 8.3, and the temperature is typically at least about 30° C. Stringent conditions can also be achieved with the addition of destabilizing agents such as formamide.

In general, a signal to noise ratio of 2× or 2.5×-5× (or higher) than that observed for an unrelated probe in the particular hybridization assay indicates detection of a specific hybridization. Detection of at least stringent hybridization between two sequences in the context of the present invention indicates relatively strong structural similarity or homology to, e.g., the nucleic acids of the present invention provided in the sequence listings herein.

As noted, “highly stringent” conditions are selected to be about 5° C. or less lower than the thermal melting point (Tₘ) for the specific sequence at a defined ionic strength and pH. Target sequences that are closely related or identical to the nucleotide sequence of interest (e.g., “probe”) can be identified under highly stringency conditions. Lower stringency conditions are appropriate for sequences that are less complementary. See, e.g., Rapley and Walker; Sambrook, Goeddel, all supra.

Comparative hybridization can be used to identify nucleic acids of the invention, and this comparative hybridization method is a preferred method of distinguishing nucleic acids of the invention. Detection of highly stringent hybridization between two nucleotide sequences in the context of the present invention indicates relatively strong structural similarity/homology to, e.g., the nucleic acids provided in the sequence listing herein. Highly stringent hybridization between two nucleotide sequences demonstrates a degree of similarity or homology of structure, nucleotide base composition, arrangement or order that is greater than that detected by stringent hybridization conditions. In particular, detection of highly stringent hybridization in the context of the present invention indicates strong structural similarity or structural homology (e.g., nucleotide structure, base composition, arrangement or order) to, e.g., the nucleic acids provided in the sequence listings herein. For example, it is desirable to identify test nucleic acids, which hybridize to the exemplar nucleic acids herein under stringent conditions.

Thus, one measure of stringent hybridization is the ability to hybridize to one of the listed nucleic acids of the invention (e.g., nucleic acid sequences of any of SEQ ID NOS:1-5, and complementary polynucleotide sequences thereof) under highly stringent conditions (or very stringent conditions, or ultra-high stringency hybridization conditions, or ultra-ultra high stringency hybridization conditions). Stringent hybridization (including, e.g., highly stringent, ultra-high stringency, or ultra-ultra high stringency hybridization conditions) and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria is met. For example, the hybridization and wash conditions are gradually increased until a probe comprising the polynucleotide sequence of SEQ ID NO:1, and complementary polynucleotide sequence thereof, binds to a perfectly matched complementary target (again, a nucleic acid comprising the polynucleotide sequence of SEQ ID NO:1, and complementary polynucleotide sequence thereof), with a signal to noise ratio that is at least 2.5×, and optionally 5× or more as high as that observed for hybridization of the probe to an unmatched target. Higher signal to noise ratios can be selected, e.g., about 10×, about 20×, about 30×, about 50× or more. The particular signal depends on the label used in the relevant assay, e.g., a fluorescent label, colorimetric label, radio active label, or the like.

A test nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 2.5×-10×, typically 5×-10× as high as that observed for hybridization to any of the unmatched target nucleic acids.

In one aspect, the invention provides a target nucleic acid that hybridizes under at least stringent or highly stringent conditions to a unique coding polynucleotide that is unique compared to a known polynucleotide, e.g., as shown in GenBank. For some such nucleic acids, the stringent conditions are selected such that a perfectly complementary polynucleotide to the coding polynucleotide hybridizes to the coding polynucleotide with at least about a 5× higher signal to noise ratio than for hybridization of the perfectly complementary oligonucleotide to a control nucleic acid, where the control nucleic acid is a known nucleic, e.g., as shown in GenBank.

Ultra high-stringency hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Target nucleic acids, which hybridize to the nucleic acid represented by any of SEQ ID NOS:1-5, or any complement thereof, under high, ultra-high and ultra-ultra high stringency conditions are a feature of the invention. Examples of such nucleic acids include those with one or a few silent or conservative nucleic acid substitutions as compared to a given nucleic acid sequence.

Nucleic acids that do not hybridize to each other under stringent conditions are still substantially identical if the polypeptides that they encode are substantially identical. This occurs, e.g., when a copy of a nucleic acid is created using the maximum codon degeneracy permitted by the genetic code, or when antisera generated against, e.g., one or more of SEQ ID NOS:3 and 4, which has been subtracted using the polypeptides encoded by known or existing polypeptide sequences or the like.

Additionally, for distinguishing between duplexes with sequences of less than about 100 nucleotides, a TMAC1 hybridization procedure known to those of skill in the art can be used. See, e.g., Sorg, U. et al. 1 Nucleic Acids Res. (Sep. 11, 1991) 19(17), incorporated herein by reference in its entirety for all purposes.

In one aspect, the invention provides a nucleic acid, which comprises a unique subsequence in any of SEQ ID NOS:1-5, wherein the unique subsequence is unique as compared to a known nucleic acid (see, e.g., sequences provided GenBank). Such unique subsequences can be determined by aligning SEQ ID NO:1 against the complete set of nucleic acids, or other sequences available, e.g., in a public database, at the filing date of the subject application. Alignment can be performed using the BLAST algorithm set to default parameters. Any unique subsequence is useful, e.g., as a probe to identify the nucleic acids of the invention.

Note that where the sequence corresponds to a non-translated sequence such as a pseudo-gene, the corresponding polypeptide is generated simply by in silico translation of the nucleic acid sequence into an amino acid sequence, where the reading frame is selected to correspond to the reading frame of homologous exogenous nucleic acids. Such polypeptides are optionally made by synthetic or recombinant approaches, or can even be ordered from companies specializing in polypeptide production.

Percent Sequence Identity—Sequence Similarity

The degree to which one nucleic acid is similar to another provides an indication of whether there is an evolutionary relationship between the two or more nucleic acids. In particular, where a high level of sequence identity is observed, it is inferred that the nucleic acids are derived from a common ancestor (i.e., that the nucleic acids are homologous). In addition, sequence similarity implies similar structural and functional properties for the two or more nucleic acids and the sequences they encode. Accordingly, in the context of the present invention, sequences that have a similar sequence to any given exemplar sequence are a feature of the present invention. In particular, sequences that have share percent sequence identities as defined below are a feature of the invention.

A variety of methods of determining sequence relationships can be used, including manual alignment and computer assisted sequence alignment and analysis. This later approach is a preferred approach in the present invention, due to the increased throughput afforded by computer-assisted methods. A variety of computer programs for performing sequence alignment are available, or can be produced by one of skill.

As noted above, the sequences of the nucleic acids and polypeptides (and fragments thereof) employed in the subject invention need not be identical, but can be substantially identical (or substantially similar), to the corresponding sequence of an exogenous polypeptide or nucleic acid molecule (or fragment thereof) or related molecule. For example, the polynucleotides or polypeptides can be subject to various changes, such as one or more amino acid or nucleic acid insertions, deletions, and substitutions, either conservative or non-conservative, including where, e.g., such changes might provide for certain advantages in their use, e.g., in their therapeutic or prophylactic use or administration or diagnostic application. The nucleic acids can also be subject to various changes, such as one or more substitutions of one or more nucleic acids in one or more codons such that a particular codon encodes the same or a different amino acid, resulting in either a conservative or non-conservative substitution, or one or more deletions of one or more nucleic acids in the sequence. The nucleic acids can also be modified to include one or more codons that provide for optimum expression in an expression system (e.g., mammalian cell or mammalian expression system), while, if desired, said one or more codons still encode the same amino acid(s). Such nucleic acid changes might provide for certain advantages in their therapeutic or prophylactic use or administration, or diagnostic application. The nucleic acids and polypeptides can be modified in a number of ways so long as they comprise a sequence substantially identical (as defined below) to a sequence in a respective exogenous nucleic acid or polypeptide molecule.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv Appl Math 2:482, by the homology alignment algorithm of Needleman and Wunsch (1970) J Mol Biol 48:443, by the search for similarity method of Pearson and Lipman (1988) Proc Natl Acad Sci USA 85:2444, by computerized implementations of these algorithms (GAP, BESTFIT, FASTA and TFASTA in the Wisconsin Genetics Software Package Release 7.0, Genetics Computer Group, 575 Science Dr., Madison, Wis.; and BLAST, see, e.g., Altschul et al. (1977) Nuc Acids Res 25:3389-3402 and Altschul et al. (1990) J Mol Biol 215:403-410), or by inspection, with the best alignment (i.e., resulting in the highest percentage of sequence similarity or sequence identity over the comparison window) generated by the various methods being selected.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence, as measured using one of the following sequence comparison algorithms or by visual inspection.

The term “sequence identity” or “percent identity” (“% identity”) means that two polynucleotide or polypeptide sequences are identical (i.e., on a nucleotide-by-nucleotide basis or amino acid-by-amino acid basis, respectively) over a window of comparison. The term “percentage of sequence identity” (or “percent sequence identity” or simply “percent identity” or “% identity”) or “percentage of sequence similarity” (or “percent sequence similarity” or simply “percent similarity”) is calculated by comparing two optimally aligned polynucleotide or polypeptide sequences over the window of comparison, determining the number of positions at which the identical residues occur in both sequences to yield the number of matched positions, dividing the number of matched positions by the total number of positions in the window of comparison (i.e., the window size), and multiplying the result by 100 to yield the percentage of sequence identity (or percentage of sequence similarity). Thus, for example, with regard to polypeptide sequences, the term sequence identity means that two polypeptide sequences are identical (on an amino acid-by-amino acid basis) over a window of comparison, and a percentage of amino acid residue sequence identity (or percentage of amino acid residue sequence similarity), can be calculated. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Maximum correspondence can be determined by using one of the sequence algorithms described herein (or other algorithms available to those of ordinary skill in the art) or by visual inspection.

The phrase “substantially identical” or “substantial identity” in the context of two nucleic acids or polypeptides, refers to two or more sequences or subsequences that have at least about 50%, 60%, 70%, 75%, preferably 80% or 85%, more preferably 90%, 91%, 92%, 93%, 94%, 95%, 96%, 97%, 98%, 99%, 99.5%, or more nucleotide or amino acid residue % identity, respectively, when compared and aligned for maximum correspondence, as measured using one of the following sequence comparison algorithms or by visual inspection. In certain embodiments, the substantial identity exists over a region of amino acid sequences that is at least about 50 residues in length, preferably over a region of at least about 100 residues in length, and more preferably the sequences are substantially identical over at least about 150, 200, or 250 amino acid residues. In certain aspects, substantial identity exists over a region of nucleic acid sequences of at least about 500 residues, preferably over a region of at least about 600 residues in length, and more preferably the sequences are substantially identical over at least about 700, 800, or 850 nucleic acid residues. In some aspects, the amino acid or nucleic acid sequences are substantially identical over the entire length of the corresponding coding region.

As applied to polypeptides and peptides, the term “substantial identity” typically means that two polypeptide or peptide sequences, when optimally aligned, such as by the programs GAP or BESTFIT using default gap weights (described in detail below) or by visual inspection, share at least about 60% or 70%, often at least 75%, preferably at least about 80% or 85%, more preferably at least about 90%, 91%, 92%, 93%, 94%, 95%, 96%, 97%, 98%, 99%, or 99.5% or more percent amino acid residue sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share at least about 60 percent, 70 percent, or 80 percent sequence identity or sequence similarity, preferably at least about 90 percent amino acid residue sequence identity or sequence similarity, more preferably at least about 95 percent sequence identity or sequence similarity, or more (including, e.g., about 90, 91, 92, 93, 94, 95, 96, 97, 98, 98.5, 99, 99.5,or more percent nucleotide sequence identity or sequence similarity).

In one aspect, the present invention provides nucleic acids having at least about 60%, 70%, 75%, 80%, 85%, 90%, 91%, 92%, 93%, 94%, 95%, 96%, 97%, 98%, 98.5%, 99%, 99.5% or more percent sequence identity or sequence similarity with the nucleic acid corresponding to any of SEQ ID NOS:1-5 or the complementary sequence thereof

Alternatively, parameters are set such that one or more sequences of the invention are identified by alignment to a query sequence, while sequences corresponding to unrelated polypeptides, e.g., those encoded by known nucleic acid sequences represented by GenBank accession numbers are not identified.

Preferably, residue positions that are not identical differ by conservative amino acid substitutions. Conservative amino acid substitution refers to the interchange-ability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having anide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, arginine-lysine-histidine, lysine-arginine, alanine-valine, and asparagine-glutamine.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv Appl Math 2:482, by the homology alignment algorithm of Needleman and Wunsch (1970) J Mol Biol 48:443, by the search for similarity method of Pearson and Lipman (1988) Proc Natl Acad Sci USA 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, with the best alignment (i.e., -resulting in the highest percentage of sequence similarity over the comparison window) generated by the various methods being selected.

A preferred example of an algorithm that is suitable for determining percent sequence identity (percent identity) and sequence similarity is the FASTA algorithm, which is described in Pearson, W. R. & Lipman, D. J. (1988) Proc Natl Acad Sci USA 85:2444. See also, W. R. Pearson (1996) Methods Enzymology 266:227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Other preferred examples of algorithm that are suitable for determining percent sequence identity and sequence similarity are the BLAST and BLAST 2.0 algorithms, which are described in Altschul et al. (1997) Nuc Acids Res 25:3389-3402 and Altschul et al. (1990) J Mol Biol 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program (e.g., BLASTP 2.0.14; Jun.-29-2000) uses as defaults a wordlength of 3, and expectation (E) of 10, and the BLOSUM62 scoring matrix (see, Henikoff & Henikoff (1989) Proc Natl Acad Sci USA 89:10915) uses alignments (B) of 50, expectation (E) of 10, M=5, N=−4, and a comparison of both strands. Again, as with other suitable algorithms, the stringency of comparison can be increased until the program identifies only sequences that are more closely related to those in the sequence listings herein (i.e., SEQ ID NOS:1-5, rather than sequences that are more closely related to other similar sequences such as, e.g., those nucleic acid sequences represented by GenBank accession numbers set forth herein, and or other similar molecules found in, e.g., GenBank. In other words, the stringency of comparison of the algorithms can be increased so that all known prior art (e.g., those represented by GenBank accession numbers shown herein, or other similar molecules found in, e.g., GenBank) is excluded.

The BLAST algorithm also performs a statistical analysis of the similarity or identity between two sequences (see, e.g., Karlin & Altschul (1993) Proc Natl Acad Sci USA 90:5873-5787). One measure of similarity provided by this algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle (1987) J Mol Evol 35:351-360. The method used is similar to the method described by Higgins & Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc Acids Res 12:387-395).

Another preferred example of an algorithm that is suitable for multiple DNA and amino acid sequence alignments is the CLUSTALW program (Thompson, J. D. et al. (1994) Nuc Acids Res 22:4673-4680). CLUSTALW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff (1992) Proc Natl Acad Sci USA 89:10915-10919). Another example of an algorithm suitable for multiple DNA and amino acid sequence alignments is the Jotun Hein method, Hein (1990), from within the MegaLine™ DNASTAR package (MegaLine™ Version 4.03, manufactured by DNASTAR, Inc.) used according to the manufacturer's instructions and default values specified in the program.

It will be understood by one of ordinary skill in the art, that the above discussion of search and alignment algorithms also applies to identification and evaluation of polynucleotide sequences, with the substitution of query sequences comprising nucleotide sequences, and where appropriate, selection of nucleic acid databases.

Diversity Generation

In general, nucleic acids and proteins derived by mutation, recursive sequence recombination (RSR) or other alterations of the nucleic acid and protein sequences described herein, respectively, are a feature of the invention. Similarly, those produced by recombination, including RSR, are a feature of the invention. Mutation and recombination methods using the nucleic acids described herein are a feature of the invention. For example, one method of the invention includes recombining one or more nucleic acids described herein with one or more additional nucleic acids (including, but not limited to those noted herein). The recombining steps are optionally performed in vivo, ex vivo, or in vitro. Also included in the invention are a recombinant nucleic acid produced by this method, a cell containing the recombinant nucleic acid, a nucleic acid library produced by this method comprising recombinant polynucleotides, and a population of cells containing the library comprising recombinant polynucleotides.

A variety of diversity generating protocols for generating and identifying molecules having one of more of the properties described herein are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics. While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are generated or produced can be selected for a desired activity or property. This can include identifying any activity that can be detected, for example, in an automated or automatable format, by any of the assays in the art and/or the assays of the invention discussed here and/or in the Example section below. A variety of related (or even unrelated) properties can be evaluated, in serial or in parallel, at the discretion of the practitioner.

Descriptions of a variety of diversity generating procedures for generating modified nucleic acid sequences, including sequences that represent modifications of nucleic acid vector sequences described herein and fragments thereof (including, e.g., nucleic acid vectors of the invention that further comprise one or more exogenous polynucleotide sequences that encode one or more therapeutic or prophylactic polypeptides of interest), as described herein are found in the following publications and the references cited therein: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438;.Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor “headpiece dimer” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

The term “shuffling” is used herein to indicate recombination between non-identical sequences; in some embodiments shuffling may include crossover via homologous recombination or via non-homologous recombination, such as via cre/lox and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using Ml 3 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” PCT/US00/26708 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

Several different general classes of sequence modification methods, such as mutation, recombination, etc. are applicable to the present invention and set forth, e.g., in the references above and below. Nucleic acids of the invention, including, e.g., components or fragments of the vectors of the invention, can be diversified by any of the methods described herein, e.g., including various mutation and recombination methods, individually or in combination, to generate nucleic acids with a desired activity or property, including, e.g., those described herein, such as an ability to enhance an immune response, such as by inducing T cell activation or proliferation, an ability to down-regulate or inhibit an immune response, such as by inhibiting T cell activation or proliferation, an ability to preferentially bind and/or signal through either or both CD28 and CTLA-4 receptors, an ability to induce production of antibodies to a self-antigen (such as, e.g., EpCAM).

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library of enriched sequences that hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutational methods that result in the alteration of individual nucleotides or groups of contiguous or non-contiguous nucleotides can be favorably employed to introduce nucleotide diversity. For example, mutagenesis procedures resulting in changes of one or more nucleotides can be used to generate any number of nucleic acids encoding polypeptides of the present invention. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11 -15 and Caldwell et al. (1992) PCR Methods Applic. 2:28-33. Similarly, assembly PCR can be used, in a process that involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above. “Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods, may be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes,” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above-described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides. A recombinant nucleic acid produced by recombining one or more polynucleotide sequences of the invention with one or more additional nucleic acids using any of the above-described formats alone or in combination also forms a part of the invention. The one or more additional nucleic acids may include another nucleic acid of the invention.

Methods of the Invention

Methods of Production and Expression

Methods for expressing or producing the heterologous polypeptides described herein using the nucleic acids or expression vectors of the invention are also a feature of the invention. One such method comprises introducing into or contacting a population of cells any nucleic acid or vector described herein, which nucleic acid or vector includes at least one polynucleotide sequence encoding a polypeptide that is operatively linked to a regulatory sequence effective to express or produce the encoded polypeptide (or introducing or contacting a population of cells with a vector described herein), and culturing the cells in a culture medium under condition suitable for expression or production of the polypeptide. Optionally, the expressed polypeptide can be isolated from the cells or from the culture medium using techniques well known in the art.

Another such method comprises introducing into a population of cells a recombinant or synthetic nucleic acid or expression vector of the invention; administering the nucleic acid or expression vector into a mammal; and isolating the polypeptide from the mammal or from a byproduct of the mammal.

The invention also includes methods for expression of at least one polypeptide of interest, where the nucleotide coding sequence encoding at least one polypeptide of interest is incorporated into the vector. Some such methods comprise introduction of at least one vector of the invention into a host cell or population of such cells. Typically, such vector comprises a promoter, terminator, and a heterologous nucleotide coding sequence encoding the polypeptide of interest that is operably linked to the promoter. The promoter directs the synthesis of encoded polypeptide, and the host cell is cultured under conditions suitable for expression of the polypeptide. Methods for expression or production of bicistronic and/or monocistronic expression vectors of the invention are a feature of the invention.

In one aspect, the invention provides a method for expressing a polypeptide, comprising: (a) providing a cell comprising at least one vector of the invention, the at least one vector further comprising a polynucleotide coding sequence that encodes the polypeptide, wherein the polynucleotide coding sequence is operably linked to a regulatory or promoter sequence that directs synthesis or expression of the polypeptide; and (b) culturing said cell under conditions suitable for expression of the polypeptide. For example, the vector may comprise a polynucleotide sequence having at least about 90, 91, 92, 93, 94, 95, 96, 97, 98, 99, 99.5% or more sequence identity to a polynucleotide sequence selected from the group consisting of SEQ ID NOS:1-5.

In another aspect, the invention includes a method for transfecting a cell or population of cells, the method comprising contacting the cell or population of cells with at least one vector of the invention under conditions for transfection of the cell with said vector.

Also included is method of expressing a polypeptide, the method comprising: (a) introducing into a population of cells at least one nucleic acid of the invention that further comprises a polynucleotide sequence that encodes the polypeptide, said polynucleotide sequence operatively linked to a regulatory sequence effective to produce the encoded polypeptide; and (b) culturing the cells in a culture medium to express the polypeptide. Some such methods further comprise isolating the polypeptide from the cells or from the culture medium.

In another aspect, the invention provides a method of producing a polypeptide, the method comprising: (a) introducing into a population of cells at least one expression vector of the invention that further comprises a polynucleotide sequence that encodes the polypeptide, wherein the polynucleotide sequence operatively linked to a promoter sequence within the nucleic acid to produce the encoded polypeptide; (b) administering the expression vector into a mammal; and (c) isolating the polypeptide from the mammal or from a byproduct of the mammal.

Therapeutics and Prophylactic Applications

The nucleic acids and vectors of the invention have properties that are of beneficial use in a variety of applications, including, e.g., but not limited to, as genetic vaccines (e.g., DNA-based vaccinations), in gene therapies, and in prophylactic and therapeutic therapies or treatments where manipulation or modulation of an immune response (e.g., inducing, enhancing or suppressing an immune response in a subject), such as manipulation or modulation of T cell activation or proliferation, antibody production, and/or cytokine production, is desirable.

The vectors and nucleic acids of invention are useful in a method of treating a disease, disorder or medical condition, wherein an effective amount of the agent (e.g., therapeutic, prophylactic, or pharmaceutical agent, protein, nucleic acid, etc.) to treat the disease, disorder or condition is delivered to a subject directly or indirectly by using the vector. Diseases, disorders, and/or medical condition treatable by administration of nucleic acids vectors of the invention which further comprise at least one appropriate polynucleotide sequence that encodes a therapeutic or prophylactically relevant polypeptide (relevant to treatment or prevention of the disease) include, but are not limited to, e.g., chronic disease, autoimmune disorder, multiple sclerosis, rheumatoid arthritis, lupus erythematosus, type I diabetes, psoriasis, diseases associated with human respiratory syncytial virus (HRSV), AIDS or AIDS-related complexes, allogeneic or xenogeneic grafts or transplants, a variety of tumor-associated diseases and cancers and (e.g., colorectal, breast, lung, prostate, and cancers associated with expression of EpCAM antigen, and other cancer diseases described herein) viral infections (e.g., hepatitis A, B, or C infection, dengue virus infection, flaviviral infection, e.g., dengue virus infection, alphavirus infection, e.g., Japanese encephalitis (JEV), Western equine encephalitis (WEE), Eastern equine encephalitis (EEE) or Venezuelan encephalitis (VEE) infection), parasitic infections, malaria, HIV, and/or bacterial infections, allergic responses (e.g., responses to dust mite or grass pollen allergens), and the like.

In one aspect, the invention also provides methods in which a nucleic acid vector of the invention is administered to a subject, such as an animal or human. In some such methods, when the nucleotide coding sequence encodes an immunogenic or antigenic polypeptide, an immune response may be induced in the subject following administration of the vector. The type of immune response that is generated in the subject will depend upon the nature of the polypeptide that is expressed. Immune responses include humoral and cellular responses against infectious agents, such as viruses, bacteria, and against tumor cells.

The invention also includes a method for inducing an immune response in a subject, comprising: administering to the subject at least one nucleic acid of the invention, wherein said nucleic acid comprises a mammalian promoter nucleotide sequence and further comprises a polynucleotide sequence encoding an antigenic polypeptide that is operatively linked to the mammalian promoter sequence, said nucleic acid being administered in an amount sufficient to induce an immune response by expression of the polypeptide.

Also provided is a method for enhancing an immune response to an antigen in a subject, comprising administering to the subject at least one of any vector of the invention, wherein each at least one vector further comprises at least one polynucleotide sequence encoding at least one immunomodulatory or co-stimulatory polypeptide, such that the immune response induced in the subject by the antigen is enhanced by the at lest one expressed immunomodulatory or c-stimulatory polypeptide, wherein the at least one immunomodulatory or co-stimulatory polypeptide is expressed and enhanced the immune response in the subject induced by an antigen. In some such methods, an expression vector encoding the antigen is administered to the subject.

In another aspect, the invention provides a method of treating a disorder or disease in a mammal in need of such treatment, comprising administering to-the subject at least one nucleic acid or nucleic acid vector of the invention, wherein the at least one nucleic acid or vector further comprise a polynucleotide sequence that encodes a polypeptide useful in treating said disorder or disease, wherein the polypeptide-encoding polynucleotide sequence e is operatively linked to a mammalian promoter nucleotide sequence effective to produce the encoded polypeptide, wherein the mammalian promoter nucleotide sequence comprises a portion of the polynucleotide sequence of the nucleic acid or vector, and wherein nucleic acid or vector is administered in an amount sufficient to produce an effective amount of the polypeptide to treat said disorder or disease.

In one aspect, the invention provides a method of therapeutic or prophylactic treatment of a disease or disorder in a subject in need of such treatment, comprising administering to the subject any vector described herein comprising a nucleotide sequence encoding a polypeptide or immunogen specific for said disease or disorder, wherein the amount of polypeptide or immunogen is effective to prophylactically or therapeutically treat said disease or disorder.

In another embodiment, the invention provides a method of modulating (enhancing or suppressing) an immune response in a subject in need of such treatment, comprising administering to the subject any vector described herein comprising a nucleotide sequence encoding a co-stimulatory polypeptide, wherein the amount of polypeptide is an effective amount such that the immune response is modulated. A co-stimulatory polypeptide typically acts in association or conjunction with, or is involved with, a second molecule or with respect to an immune response in a co-stimulatory pathway. In one aspect, a co-stimulatory polypeptide may be an immunomodulatory molecule that acts in association or conjunction with, or is involved with, another molecule to stimulate or enhance an immune response. In another aspect, a co-stimulatory molecule is immunomodulatory molecule that acts in association or conjunction with, or is involved with, another molecule to inhibit or suppress an immune response. A co-stimulatory molecule need not act simultaneously with or by the same mechanism as the second molecule.

In another aspect is provide a method of modulating an immune response in a subject, comprising administering to the subject any vector described herein, wherein the vector further comprises a nucleotide sequence encoding a co-stimulatory polypeptide. The amount of expressed co-stimulatory polypeptide is an effective amount such that the immune response is modulated. In one embodiment, a nucleotide sequence encoding a co-stimulatory polypeptide that enhances an immune response, such as by inducing T cell activation or proliferation (e.g., agonists) is incorporated into a vector of the invention; alternatively, a nucleotide sequence encoding a co-stimulatory polypeptide that down-regulates or inhibits an immune response, such as by inhibiting T cell activation or proliferation (e.g., antagonists) is incorporated into a vector of the invention.

A nucleotide sequence that encodes a polypeptide that preferentially binds and/or signals through either or both the CD28 and CTLA-4 receptors may be incorporated into a vector of the invention. For example, variants, mutants, derivatives, and fragments of: 1) B7-1 and B7-2 polypeptides and nucleic acids, and 2) B7-1 and B7-2 polypeptides and nucleic acids of the Artiodactyla family (including, e.g., bovine B7-1 and 137-2), including all such polypeptide variants (and nucleic acids encoding such polypeptide variants) that exhibit properties similar or equivalent to the properties of a polypeptide that binds a CD28 receptor (e.g., a CD28 binding protein) or a polypeptide that binds a CTLA-4 receptor (e.g., a CTLA-4 binding protein (“CTLA-4BP”).

In another aspect, the invention includes a method of inducing an immune response against a pathogen, such as, e.g., a viral agent, bacterial agent, allergen, or cancer agent, which comprises administering to a subject in need of such treatment a genetic vaccine vector of the invention in an amount effective to induce a detectable immune response against the agent. In one aspect, the genetic vaccine vector comprises a DNA vaccine vector of the invention (e.g., any of SEQ ID NOS:1-5), which further comprises at least one polynucleotide sequence encoding at least one antigen. Any antigen of interest may be employed. If desired, the vaccine vector may also include at least one polynucleotide sequence encoding at least one additional polypeptide that enhances the immune response induced by the antigen (e.g., adjuvant, co-stimulator, cytokine, immunomodulator, chemokine, or the like).

In another aspect, the present invention includes methods of therapeutically or prophylactically treating a disease or disorder by administering, in vivo or ex vivo, one or more nucleic acids of the invention described above (or compositions, vectors, or transduced cells comprising a pharmaceutically acceptable excipient and one or more such nucleic acids or polypeptides) to a subject or to a population of cells of the subject, including, e.g., a mammal, including, e.g., a human, primate, monkey, orangutan, baboon, mouse, pig, cow, cat, goat, rabbit, rat, guinea pig, hamster, horse, sheep; or a non-mammalian vertebrate such as a bird (e.g., a chicken or duck) or a fish, or invertebrate.

In one aspect of the invention, in ex vivo methods, one or more cells or a population of cells of interest of the subject (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) are obtained or removed from the subject and contacted with an amount of a polypeptide of the invention that is effective in prophylactically or therapeutically treating a disease, disorder, or other condition. The contacted cells are then returned or delivered to the subject to the site from which they were obtained or to another site (e.g., including those defined above) of interest in the subject to be treated. If desired, the contacted cells may be grafted onto a tissue, organ, or system site (including all described above) of interest in the subject using standard and well-known grafting techniques or, e.g., delivered to the blood or lymph system using standard delivery or transfusion techniques.

The invention also provides in vivo methods in which at least one cell or a population of cells of interest of the subject are contacted directly or indirectly with a sufficient amount of a nucleic acid of the invention (which optionally comprises at least one exogenous polynucleotide sequence encoding a polypeptide of interest (e.g., antigen, co-stimulatory polypeptide, adjuvant, and/or cytokine, etc.) effective in prophylactically or therapeutically treating a disease, disorder, or other condition. In direct (e.g., local) contact or administration formats, the polypeptide is typically administered or transferred directly (e.g., locally) to the cells to be treated or to the tissue site of interest (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) by any of a variety of formats, including topical administration, injection (e.g., using a needle or syringe), or vaccine or gene gun delivery, or pushing into a tissue, organ, or skin site.

The nucleic acids of the invention can be delivered by a variety of routes, e.g., intramuscularly, intradermally, subdermally, subcutaneously, orally, intraperitoneally, intrathecally, intravenously, mucosally, systemically, parenterally, via inhalation, or placed within a cavity of the body (including, e.g., during surgery), or by inhalation or vaginal or rectal administration.

In in vivo and ex vivo indirect contact/administration formats, the nucleotide acid (or polypeptide encoded therefrom) is typically administered or transferred indirectly to the cells to be treated or to the tissue site of interest, including those described above (such as, e.g., skin cells, organ systems, lymphatic system, or blood cell system, etc.), by contacting or administering the nucleic acid of the invention (or polypeptide encoded therefrom) directly to one or more cells or population of cells from which treatment can be facilitated. For example, tumor cells within the body of the subject can be treated by contacting cells of the blood or lymphatic system, skin, or an organ with a sufficient amount of the polypeptide such that delivery of the polypeptide to the site of interest (e.g., tissue, organ, or cells of interest or blood or lymphatic system within the body) occurs and effective prophylactic or therapeutic treatment results. Such contact, administration, or transfer is typically made by using one or more of the routes or modes of administration described above.

In another aspect, the invention provides ex vivo methods in which one or more cells of interest or a population of cells of interest of the subject (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) are obtained or removed from the subject and transformed by contacting said one or more cells or population of cells with a polynucleotide construct comprising a target nucleic acid sequence of the invention or fragments thereof, that encodes a biologically active polypeptide of interest (e.g., a polypeptide of the invention) that is effective in prophylactically or therapeutically treating the disease, disorder, or other condition. The one or more cells or population of cells is contacted with a sufficient amount of the polynucleotide construct and a promoter controlling expression of said nucleic acid sequence such that uptake of the polynucleotide construct (and promoter) into the cell(s) occurs and sufficient expression of the target nucleic acid sequence of the invention results to produce an amount of the biologically active polypeptide effective to prophylactically or therapeutically treat the disease, disorder, or condition. The polynucleotide construct may include a promoter sequence (e.g., WT, recombinant, or chimeric CMV promoter sequence) that controls expression of a component of a nucleic acid vector of the invention (e.g., exogenous polynucleotide) and/or, if desired, one or more additional exogenous nucleotide sequences encoding at least one additional exogenous polypeptide (e.g., cytokine, adjuvant, antigen, or a co-stimulatory polypeptide, or other polypeptide of interest).

Following transfection, the transformed cells are returned, delivered, or transferred to the subject to the tissue site or system from which they were obtained or to another site (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) to be treated in the subject. If desired, the cells may be grafted onto a tissue, skin, organ, or body system of interest in the subject using standard and well-known grafting techniques or delivered to the blood or lymphatic system using standard delivery or transfusion techniques. Such delivery, administration, or transfer of transformed cells is typically performed or made by using one or more of the routes or modes of administration described above. Expression of the target nucleic acid occurs naturally or can be induced (as described in greater detail below) and an amount of the encoded polypeptide is expressed sufficient and effective to treat the disease or condition at the site or tissue system.

In another aspect, the invention provides in vivo methods in which one or more cells of interest or a population of cells of the subject (e.g., including those cells and cell(s) systems and subjects described above) are transformed in the body of the subject by contacting the cell(s) or population of cells with (or administering or transferring to the cell(s) or population of cells using one or more of the routes or modes of administration described above) a polynucleotide construct comprising a nucleic acid sequence of the invention that encodes a biologically active polypeptide of interest (e.g., a polypeptide of the invention) that is effective in prophylactically or therapeutically treating the disease, disorder, or other condition.

The polynucleotide construct can be directly administered or transferred to cell(s) exhibiting or having the disease or disorder (e.g., by direct contact using one or more of the routes or modes of administration described above). Alternatively, the polynucleotide construct can be indirectly administered or transferred to cell(s) exhibiting or having the disease or disorder by first directly contacting non-diseased cell(s) or other diseased cells using one or more of the routes or modes of administration described above with a sufficient amount of the polynucleotide construct comprising the nucleic acid sequence encoding the biologically active polypeptide, and a promoter controlling expression of the nucleic acid sequence, such that uptake of the polynucleotide construct (and promoter) into the cell(s) occurs and sufficient expression of the nucleic acid sequence of the invention results to produce an amount of the biologically active polypeptide effective to prophylactically or therapeutically treat the disease or disorder, and whereby the polynucleotide construct or the resulting expressed polypeptide is transferred naturally or automatically from the initial delivery site, system, tissue or organ of the subject's body to the diseased site, tissue, organ or system of the subject's body (e.g., via the blood or lymphatic system). Expression of the target nucleic acid occurs naturally or can be induced (as described in greater detail below) such that an amount of the encoded polypeptide expressed is sufficient and effective to treat the disease or condition at the site or tissue system. The polynucleotide construct may include a promoter sequence (e.g., wild-type, recombinant or chimeric CMV promoter sequence) that controls expression of the nucleic acid sequence and/or, if desired, one or more additional nucleotide sequences encoding at least one additional exogenous polypeptide of interest.

In one aspect, tumor cells of a patient are transfected with a DNA plasmid vector encoding a polypeptide of interest (e.g., CD28BP) to facilitate an improved immune response, (e.g., enhanced T cell response or increased antibody titer). The tumor cells may be removed from the patient and transfected ex vivo, and then re-delivered to the patient, preferably at the tumor site. Alternatively, the tumor cells of a tumor are transfected in vivo, by delivering a DNA plasmid encoding a CD28BP polypeptide of interest. In either case, the immune response can be measured by measuring T cell proliferation using methods described herein or antibody levels using standard protocols. In another aspect, a DNA plasmid encoding a soluble CD28BP or soluble CD28BP-Ig is administered to a patient by any means described herein, including systemically, subcutaneously, intramuscularly (i.m.), intradermally (i.d.), etc. and the like, via a needle or gene gun or other introduction mechanism described herein; if desired, the plasmid is introduced directly into cells of a tumor or tumor-related cells of the patient.

The nucleic acids of the invention are also useful as vaccine adjuvants in vaccine applications as discussed herein and for diagnostic purposes, as for in vitro applications for testing and diagnosing such diseases.

In one embodiment, the invention provides an expression vector comprising a polynucleotide encoding a CD28 receptor binding protein (e.g., CD28BP-15 as described herein) (or fragment thereof, such as the extracellular domain, or fusion protein including CD28BP-15) to enhance the properties of a DNA vaccine. For example, the CD28BP-encoding sequence may serve to non-specifically enhance the immune response of the subject to the antigen of interest, which is also administered to the subject. The expression vector can further include a polynucleotide sequence encoding an antigen of interest for which the immune response is to be enhanced by the CD28BP polypeptide.

In each of the in vivo and ex vivo treatment methods as described above, a composition comprising an excipient and the nucleic acid of the invention can be administered or delivered. In one aspect, a composition comprising a pharmaceutically acceptable excipient (e.g., PBS) and a nucleic acid of the invention, which further comprises a polynucleotide sequence that encodes a therapeutic or prophylactic polypeptide of interest (e.g., antigen, co-stimulatory polypeptide, cytokine, adjuvant etc.) is administered or delivered to the subject as described above in an amount effective to treat the disease or disorder.

In another aspect, in each in vivo and ex vivo treatment method described above, the amount of polynucleotide administered to the cell(s) or subject can be an amount sufficient that uptake of said polynucleotide into one or more cells of the subject occurs and sufficient expression of said nucleic acid sequence results to produce an amount of a biologically active polypeptide effective to enhance an immune response in the subject, including an immune response induced by an immunogen (e.g., antigen). In another aspect, for each such method, the amount of polypeptide administered to cell(s) or subject can be an amount sufficient to enhance an immune response in the subject, including that induced by an immunogen (e.g., antigen).

In yet another aspect, in each in vivo and ex vivo treatment method described above, the amount of polynucleotide administered to the cell(s) or subject can be an amount sufficient that uptake of said polynucleotide into one or more cells of the subject occurs and sufficient expression of said nucleic acid sequence results to produce an amount of a biologically active polypeptide effective to produce a tolerance or anergy response in the subject. In another aspect, for each such method, the amount of polypeptide administered to cell(s) or subject can be an amount sufficient to produce a tolerance or anergy response in the subject.

The amount of DNA plasmid for use in such methods where administration is by injection is from about 50 micrograms (ug) to 5 mg, usually about 100 ug to about 2.5 mg, typically about 500 ug to 2 mg or about 800 ug to about 1.5 mg, and often about 1 mg. The amount of DNA plasmid for use in these methods where administration is via a gene gun, e.g., is from about 100 to 1000 times less; thus, for each range given above for DNA plasmid administration via injection, the range for DNA plasmid administration via gene gun is about 100 to 1000 times less. For example, for gene gun delivery, the amount of DNA plasmid corresponding to the first range above is from about 50×10⁻⁸ g to 5×10⁻⁵ g (100 times less) or from about 50×10⁻⁹ to about 5×10⁻⁶ g. DNA plasmid amounts can be readily adjusted by those of ordinary skill in the art based upon responses in animal models obtained using the DNA plasmid vector encoding WT hB7-1 and/or antigen or based upon known DNA vaccination studies using plasmid vectors encoding a mammalian B7-1, such as WT hB7-1. Such amounts of DNA plasmid can be used, if desired, in the method in Example VI.

In yet another aspect, in an in vivo or in vivo treatment method in which a polynucleotide construct (or composition comprising a polynucleotide construct) is used to deliver a physiologically active polypeptide to a subject, the expression of the polynucleotide construct can be induced by using an inducible on- and off-gene expression system. Examples of such on- and off-gene expression systems include the Tet-On™ Gene Expression System and Tet-Off™ Gene Expression System (see, e.g., Clontech Catalog 2000, pg. 110-111 for a detailed description of each such system), respectively. Other controllable or inducible on- and off-gene expression systems are known to those of ordinary skill in the art. With such system, expression of the target nucleic of the polynucleotide construct can be regulated in a precise, reversible, and quantitative manner. Gene expression of the target nucleic acid can be induced, for example, after the stable transfected cells containing the polynucleotide construct comprising the target nucleic acid are delivered or transferred to or made to contact the tissue site, organ or system of interest. Such systems are of particular benefit in treatment methods and formats in which it is advantageous to delay or precisely control expression of the target nucleic acid (e.g., to allow time for completion of surgery and/or healing following surgery; to allow time for the polynucleotide construct comprising the target nucleic acid to reach the site, cells, system, or tissue to be treated; to allow time for the graft containing cells transformed with the construct to become incorporated into the tissue or organ onto or into which it has been spliced or attached, etc.).

Genetic Vectors

Gene therapy and genetic vaccine vectors are useful for treating and/or preventing various diseases and other conditions. The following discussion focuses on the on the use of vectors because gene therapy and genetic vaccine method typically employ vectors, but persons of skill in the art appreciate that the nucleic acids of the invention can, depending on the particular application, be employed in the absence of vector sequences. Accordingly, references in the following discussion to vectors should be understood as also relating to nucleic acids of the invention that lack vector sequences. The invention includes vectors comprising one or more nucleic acids of the invention, including nucleic acids encoding exogenous polypeptides of interest.

Vectors can be delivered to a subject to induce an immune response or other therapeutic or prophylactic response. Suitable subjects include, but are not limited to, a mammal, including, e.g., a human, primate, monkey, orangutan, baboon, mouse, pig, cow, cat, goat, rabbit, rat, guinea pig, hamster, horse, sheep; or a non-mammalian vertebrate such as a bird (e.g., a chicken or duck) or a fish, or invertebrate.

Vectors can be delivered in vivo by administration to an individual patient, typically by local (direct) administration or by systemic administration (e.g., intravenous, intraperitoneal, intramuscular, subdermal, intracranial, anal, vaginal, oral, mucosal, inhalation, systemic, parenteral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In local (direct) administration formats, the nucleic acid or vector is typically administered or transferred directly to the cells to be treated or to the tissue site of interest (e.g., tumor cells, tumor tissue sample, organ cells, blood cells, cells of the skin, lung, heart, muscle, brain, mucosac, liver, intestine, spleen, stomach, lymphatic system, cervix, vagina, prostate, mouth, tongue, etc.) by any of a variety of formats, including topical administration, injection (e.g., by using a needle or syringe), or vaccine or gene gun delivery, pushing into a tissue, organ, or skin site. For standard gene gun administration, the vector or nucleic acid of interest is precipitated onto the surface of microscopic metal beads. The microprojectiles are accelerated with a shock wave or expanding helium gas, and penetrate tissues to a depth of several cell layers. For example, the Accel™ Gene Delivery Device manufactured by Agacetus, Inc. Middleton Wis. is suitable for use in this embodiment. The nucleic acid or vector can be delivered, for example, intramuscularly, intradermally, subdermally, subcutaneously, orally, intraperitoneally, intrathecally, intravenously, mucosally, systemically, parenterally, via inhalation, or placed within a cavity of the body (including, e.g., during surgery), or by inhalation or vaginal or rectal administration.

In in vivo indirect contact/administration formats, the nucleic acid or vector is typically administered or transferred indirectly to the cells to be treated or to the tissue site of interest, including those described above (such as, e.g., skin cells, organ systems, lymphatic system, or blood cell system, etc.), by contacting or administering the nucleic acid or vector of the invention directly to one or more cells or population of cells from which treatment can be facilitated. For example, tumor cells within the body of the subject can be treated by contacting cells of the blood or lymphatic system, skin, or an organ with a sufficient amount of the polypeptide such that delivery of the nucleic acid or vector to the site of interest (e.g., tissue, organ, or cells of interest or blood or lymphatic system within the body) occurs and effective prophylactic or therapeutic treatment results. Such contact, administration, or transfer is typically performed or made by using one or more of the routes or modes of administration described above.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7):682-691; Rose U.S. Pat. No. 5,279,833; Brigham (1991) WO 91/06309; and Felgner et al. (1987) Proc. Natl Acad. Sci. USA 84:7413-7414), as well as use of viral vectors (e.g., adenoviral (see, e.g., Berns et al. (1995) Ann. NY Acad. Sci. 772:95-104; Ali et al. (1994) Gene Ther. 1:367-384; and Haddada et al. (1995) Curr. Top. Microbiol. Immunol. 199 (Pt 3):297-306 for review), papillomaviral, retroviral (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin et al. (1984) Mol. Cell. Biol., 4:2072-2081; Hermonat and Muzyczka (1984) Proc. Natl Acad. Sci. USA, 81:6466-6470; McLaughlin et al. (1988) and Samulski et al. (1989) J. Virol., 63:03822-3828), and the like.

“Naked” DNA and/or RNA that comprises a genetic vaccine can also be introduced directly into a tissue, such as muscle, by injection using a needle or other similar device. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of genetic vaccines into cells of a mammal according to the invention. These methods are useful not only for in vivo introduction of DNA into a subject, such as a mammal, but also for ex vivo modification of cells for reintroduction into a mammal. DNA is conveniently introduced directly into the cells of a mammal or other subject using, e.g., injection, such as via a needle, or a “gene gun.” As for other methods of delivering genetic vaccines, if necessary, vaccine administration is repeated in order to maintain the desired level of immunomodulation, such as the level or response of T cell activation or T cell proliferation, or antibody titer level. Alternatively, nucleotides can be impressed into the skin of the subject.

Gene therapy and genetic vaccine vectors (e.g., DNA, plasmids, expression vectors, adenoviruses, liposomes, papillomaviruses, retroviruses, etc.) of the invention comprising at least one exogenous polynucleotide sequence of interest which encodes an exogenous polypeptide (e.g., therapeutic or prophylactic polypeptide) can be administered directly to the subject (usually a mammal) for transduction of cells in vivo or ex vivo. The vectors can be formulated as pharmaceutical compositions for administration in any suitable manner, including parenteral (e.g., subcutaneous, intramuscular, intradermal, or intravenous), inhalation, mucosal, topical, oral, rectal, vaginal, intrathecal, buccal (e.g., sublingual), or local administration, such as by aerosol or transdermally, for immunotherapeutic or other prophylactic and/or therapeutic treatment. Pretreatment of skin, for example, by use of hair-removing agents, may be useful in transdermal delivery. Suitable methods of administering such packaged nucleic acids are available and well known to those of skill in the art, and, although more than one route can be used to administer a particular composition, a particular route can often provide a more immediate and more effective reaction than another route.

Further, the vectors of this invention comprising at least one nucleotide sequence encoding at least one exogenous nucleotide sequence encoding, e.g., an antigen or co-stimulatory molecule co-expressed on the same vector can be used to prophylactically or therapeutically treat or supplement such treatment of other immunological disorders and diseases or enhance protection against disorders, diseases, and antigens (including WT and recombinant antigens), e.g., in protein vaccines and DNA vaccines, including, but not limited to, e.g., allergy/asthma, neurological, organ transplantation (e.g., graft versus host disease, and autoimmune diseases), malignant diseases, chronic infectious diseases, including, but not limited to, e.g., viral infectious diseases, such as those associated with, but not limited to, e.g., alpha viruses, hepatitis viruses, e.g., hepatitis B virus (HBV), herpes simplex virus (HSV), hepatitis C virus (HCV), HIV, human papilloma virus (HPV), malaria, Venezuelan equine encephalitis (VEE), Western equine encephalitis (WEE), Japanese encephalitis virus (JEV), Eastern equine encephalitis (EEE), and the like, and bacterial infectious diseases, such as, e.g., but not limited to, e.g., Lyme disease, tuberculosis, and chlamydia infections; and other diseases and disorders described herein.

If desired, a separate vector comprising a first exogenous polynucleotide sequence encoding an exogenous polypeptide of interest (including, e.g., an antigen or co-stimulatory polypeptide) can be delivered simultaneously with a vector comprising a second exogenous polynucleotide sequence of the invention.

Compositions and Formulations

The invention also includes compositions comprising one or more nucleic acids, vectors or cells (or a population of cells) of the invention. In one aspect, the invention provides compositions comprising at least one nucleic acid or nucleic acid vector of the invention described herein and an excipient or carrier. Such composition may be a pharmaceutical composition, and the excipient or carrier may be a pharmaceutically acceptable excipient or carrier.

In a particular aspect, the invention provides compositions comprising an isolated, synthetic or recombinant nucleic acid comprising at least one polynucleotide sequence having at least about 90, 91, 92, 93, 94, 95, 96, 97, 98, 99, 99.5% or more sequence identity to a polynucleotide sequence selected from the group consisting of SEQ ID NOS:1-5, and a carrier or excipient. Preferably, the composition is a pharmaceutical composition and the excipient is pharmaceutically acceptable excipient carrier.

The invention also includes compositions produced by digesting one or more of the nucleic acids described herein with a restriction endonuclease, an RNAse, or a DNAse; and, compositions produced by incubating one or more nucleic acids described herein in the presence of deoxyribonucleotide triphosphates and a nucleic acid polymerase, e.g., a thermostable polymerase.

The invention also includes compositions comprising two or more nucleic acids of the invention described herein. The composition may comprise a library of nucleic acids, where the library contains at least 5, 10, 20, 50, 100, 500 or more nucleic acids.

The invention also includes compositions comprising at least two nucleic acids or at least two vectors of the invention and an excipient or carrier. The nucleic acid vectors of the invention thereof may be employed for therapeutic or prophylactic uses in combination with a suitable carrier, such as a pharmaceutical carrier. Such vectors typically include a heterologous coding sequence that encodes a therapeutic polypeptide of interest. Compositions comprising such vectors typically comprise a pharmaceutically acceptable carrier or excipient and amount of the vector such that a therapeutically and/or prophylactically effective amount of the polypeptide will generally be expressed in vivo in the subject to whom the vector is administered. Such a carrier or excipient includes, but is not limited to, saline, buffered saline, dextrose, water, glycerol, ethanol, and combinations thereof The formulation should suit the mode of administration. Methods of administering nucleic acids, polypeptides, and proteins are well known in the art, and are further discussed below.

The invention also includes compositions produced by digesting one or more of any of the nucleic acids or vectors described above with a restriction endonuclease, an RNAse, or a DNAse (e.g., as is performed in certain of the recombination formats noted above); and compositions produced by fragmenting or shearing one or more nucleic acid of the invention by mechanical means (e.g., sonication, vortexing, and the like), which can also be used to provide substrates for recombination in the methods described herein. The invention also provides compositions produced by cleaving at least one of any of the nucleic acids described above. The cleaving may comprise mechanical, chemical, or enzymatic cleavage, and the enzymatic cleavage may comprise cleavage with a restriction endonuclease, an RNAse, or a DNAse.

The invention also provides compositions produced by a process comprising incubating one or more of the fragmented nucleic acid sets in the presence of ribonucleotide or deoxyribonucleotide triphosphates and a nucleic acid polymerase. This resulting composition forms a recombination mixture for many of the recombination formats noted above. The nucleic acid polymerase may be an RNA polymerase, a DNA polymerase, or an RNA-directed DNA polymerase (e.g., a “reverse transcriptase”); the polymerase can be, e.g., a thermostable DNA polymerase (e.g., VENT, TAQ, or the like).

In one aspect, the invention provides therapeutic and/or prophylactic compositions comprising at least one nucleic acid of the invention or fragment thereof, vector, plasmid, or expression vector of the invention, transduced cells comprising any of nucleic acid of the invention, or vaccines comprising at least one nucleic acid (or fragment thereof) of the invention. Compositions of the invention may also include one or more additional nucleic acid sequences or segments incorporated into the nucleic acid of the invention (such as an expression vector) or combined or delivered with such nucleic acid of the invention, including, e.g., at least one nucleic acid sequence or segment encoding at least one exogenous polypeptide of interest (e.g., co-stimulatory molecule (such as, e.g., a B7-1, B7-2, a CD28 binding protein (CD28BP-15 as described herein), CTLA-4 binding protein, and the like), cytokine (e.g., GM-CSF, IL-12, IL-15, IL-18, etc. and the like), at adjuvant (CT/LT enterotoxin) and/or at least one antigen (e.g., viral antigen, such as a hepatitis B antigen, flavivirus antigen (e.g., dengue virus antigen or an antigen that protects against infection by one or more dengue viruses); bacterial antigen; cancer antigen (e.g., such as EpCAM/KSA or a variant thereof). Such compositions optionally are tested in appropriate in vitro and in vivo animal models of disease, to confirm efficacy, tissue metabolism, and to estimate dosages, according to methods well known in the art. The amount of DNA administered via a DNA vaccine (which amount includes the nucleic acid vector) depends upon the manner of delivery (e.g., via biolistic methods, injection, transdermal administration, etc.) and may range from about 0.001 mg, 0.05 mg, 0.01 mg, 0.1 mg, 0.25 mg, 0.5 mg, 1 mg, 2 mg, 2.5 mg, 5 mg, 10, 20 mg, 25 mg, 50 mg total DNA or more. One of skill can readily ascertain the total amount of nucleic acid to be administered depending upon whether the DNA is administered via biolistic methods, injection, transdermal administration, etc., and other known methods for administration of nucleic acid vectors in therapeutic and prophylactic treatment regimes and in gene therapy methods. In particular, dosages for therapeutic and prophylactic methods for treating or preventing a disease or condition can be determined by activity comparison of the molecules encoded by the nucleic acid vector to other known therapeutics using similar compositions in a relevant assay and mammalian model, including as described below.

Administration optionally is by any of the routes normally used for introducing a molecule into ultimate contact with blood or tissue cells. See, supra. The polypeptides and polynucleotides, and vectors, cells, and compositions comprising such molecules, are administered in any suitable manner, preferably with pharmaceutically acceptable carriers. Suitable methods of administering such molecules, in the context of the present invention, to a patient are available, and, although more than one route can be used to administer a particular composition, a particular route can often provide a more immediate and more effective reaction than another route. Preferred routes are readily ascertained by those of skill in the art.

Compositions comprising cells expressing at least one full length form of a pMaxVax10.1 nucleic acid or a fragment thereof are also a feature of the invention. Such cells may also express one or more antigens specific for the intended application (e.g., cancer antigen). Such cells are readily prepared as described herein by transfection with DNA plasmid vector encoding; such DNA plasmid may include at least one exogenous nucleic acid sequence encoding at least one of co-stimulatory molecule, antigen, adjuvant, cytokine and/or other exogenous polypeptide. Separate vectors encoding each such exogenous polypeptide sequence may be used to transfect the cells, or a bicistronic or multicistronic vector of the invention comprising a pMaxVax10.1 vector which further comprises two or more exogenous nucleotide sequences encoding two or more exogenous polypeptides can be used. Compositions of such cells may be pharmaceutically compositions further comprising a pharmaceutically acceptable carrier or excipient.

Pharmaceutical compositions of the invention can, but need not, include a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention. A variety of aqueous carriers can be used, e.g., buffered saline, such as PBS, and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of gene therapy or genetic vaccine vector in these formulations can vary widely, and will be selected primarily based on fluid volumes, viscosities, body weight and the like in accordance with the particular mode of administration selected and the patient's needs.

Compositions comprising polypeptides and polynucleotides, vectors, plasmids, cells, and other formulations comprising these and other components of the invention, can be administered by a number of routes including, but not limited to oral, intranasal, intravenous, intraperitoneal, intramuscular, transdermal, subcutaneous, intradermal, topical, systemic, mucosal, inhalation, parenteral, sublingual, vaginal, or rectal means. Polypeptide and nucleic acid compositions can also be administered via liposomes. Such administration routes and appropriate formulations are generally known to those of skill in the art.

The polynucleotide, nucleic acid vector, or fragment thereof of the invention, alone or in combination with other suitable components, can also be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the gene therapy vectors and genetic vaccines, when administered orally, must be protected from digestion. This is typically accomplished either by complexing the vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient.

The packaged nucleic acids, alone or in combination with other suitable -components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules that consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, subdermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, mucosally, topically, intraperitoneally, intravesically or intrathecally. The formulations of packaged nucleic acids or polypeptides of the invention can be presented in unit-dose or multi-dose sealed containers, such as ampules and vials.

Parenteral administration and intravenous administration are preferred methods of administration. In particular, any routes of administration already in use for existing co-stimulatory therapeutics and prophylactic treatment protocols, including those currently employed with e.g., other nucleic acids and nucleic acid vectors known by those of skill in the art, along with pharmaceutical compositions and formulations in current use, are also routes of administration and formulation for the nucleic acids and nucleic acid vectors (and fragments thereof) of the invention.

Injection solutions and suspensions can be prepared from sterile powders, granules, and tablets of the kind previously described. Cells transduced by the packaged nucleic acid can also be administered intravenously or parenterally.

Cells transduced with the nucleic acids as described herein in the context of ex vivo or in vivo therapy can also be administered intravenously or parenterally. It will be appreciated that the delivery of cells to patients is routine, e.g., delivery of cells to the blood via intravenous, intramuscular, or intraperitoneal administration or other common route.

The dose administered to a patient, in the context of the present invention is sufficient to effect a beneficial effect, such as an altered immune response or other therapeutic and/or prophylactic response in the patient over time, or to, e.g., inhibit infection by a pathogen, depending on the application. The dose will be determined by the efficacy of the particular nucleic acid, polypeptide, vector, composition or formulation, transduced cell, cell type, and/or the activity of the polypeptide and/or polynucleotide included therein or employed, and the condition of the patient, as well as the body weight, surface area, or vascular surface area, of the patient to be treated. The size of the dose also will be determined by the existence, nature, and extent of any adverse side-effects that accompany the administration of any such particular polypeptide, nucleic acid, vector, formulation, composition, transduced cell, cell type, or the like in a particular patient. Dosages to be used for therapeutic or prophylactic treatment of a particular disease or disorder can be determined by one of skill by comparison to those dosages used for existing therapeutic or prophylactic treatment protocols for the same disease or disorder.

Injection solutions and suspensions can be prepared from sterile powders, granules, and tablets of the kind previously described. Cells transduced by the packaged nucleic acid can also be administered intravenously or parenterally.

In determining the effective amount of the vector, cell type, composition, or formulation to be administered to a subject for the treatment or prophylaxis of the medical condition or disease state (e.g., cancers (colon, colorectal) or viral diseases (e.g., dengue virus infection and related disorders), a physician evaluates the subject for, e.g., circulating plasma levels, nucleic acid vector/cell/formulation/encoded polypeptide molecule toxicities, progression of the disease or condition, and the production of anti-vector/anti-nucleic acid/polypeptide antibodies, and depending on the subject other factors that would be known to one of skill in the art.

In one aspect, for example, in determining the effective amount of the vector to be administered in the treatment or prophylaxis of an infection or other condition, wherein the vector comprises any nucleic acid sequence described herein or encodes any polypeptide described herein, the physician evaluates vector toxicities, progression of the disease, and the production of anti-vector antibodies, if any. In one aspect, the dose equivalent of a naked nucleic acid from a vector for a typical 70 kilogram patient can range from about 10 ng to about 1 g, about 100 ng to about 100 mg, about 1 μg to about 10 mg, about 10 μg to about 1 mg, or from about 30-300 μg. Doses of vectors used to deliver the nucleic acid are calculated to yield an equivalent amount of therapeutic nucleic acid. Administration can be accomplished via single or divided doses.

In another aspect, the dose administered, e.g., to a 70 kilogram patient can be in the range equivalent to any dosages of currently-used co-stimulatory or therapeutic or prophylactic proteins or the like, and doses of vectors or cells which produce exogenous sequences optionally are calculated to yield an equivalent amount of exogenous nucleic acid or expressed polypeptide or protein. The vectors of this invention comprising at least one nucleotide sequence encoding at least one exogenous (and, if desired, further comprising at least one nucleotide sequence encoding at least one antigen, co-stimulatory molecule, cytokine, and/or adjuvant, or other exogenous polypeptide, on the same vector or on separate vectors) can be used to prophylactically or therapeutically treat or supplement such treatment of a variety of viral diseases (including, e.g., but not limited to, hepatitis A, B, and C viruses, human respiratory syncytial virus, dengue virus, Japanese encephalitis virus, Eastern equine encephalitis virus (EEE), Venezuelan equine encephalitis virus (VEE)), HIV, parasitic diseases, malaria, allergic diseases, cancers, including e.g., colorectal cancer, colon cancer, rectal cancer, breast cancer, pancreatic cancer, lung cancer, prostate cancer, naso-pharyngeal cancer, cancer, brain cancer, leukemia, melanoma, head- and/or neck cancer, stomach cancer, cervical cancer, ovarian cancer, lymphomas, colon cancer, colorectal, and virally-mediated conditions by any known conventional therapy, including cytotoxic agents, nucleotide analogues (e.g., when used for treatment of HIV infection), biologic response modifiers, and the like.

In therapeutic applications, compositions are administered to a patient suffering from a disease (e.g., an infectious disease, cancer, or autoimmune disorder) in an amount sufficient to cure or at least partially arrest or ameliorate the disease or at least one of its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of protein to effectively treat the patient.

In prophylactic applications, compositions are administered to a human or other mammal to induce an immune or other prophylactic response that can help protect against the establishment of an infectious disease, cancer, autoimmune disorder, or other condition.

In some applications, an amount of exogenous polypeptide that is administered to a subject for a particular therapeutic or prophylactic treatment protocol or vaccination ranges from about 1 to about 50 mg/kg weight of the subject. Such amount of polypeptide can be administered 1 time/week or up to 3 times/week, as desired. Such exogenous polypeptide can be administered as a soluble molecule comprising, e.g., an extracellular domain of an antigen or co-stimulatory molecule or fragment thereof. Alternatively, such exogenous polypeptide can be administered in the form of a polypeptide-encoding polynucleotide, which is operably linked to a promoter, such that the polynucleotide expresses in the subject such an exogenous polypeptide of from about 1 to about 50 mg/kg weight of the subject (e.g., on the surface of targeted cells) or as an expressed soluble exogenous polypeptide. The exogenous polypeptide (or nucleic acid encoding the polypeptide) can be administered to a population of cells of a subject in vivo, or to a population of cells of the subject ex vivo as described herein. Compositions comprising soluble exogenous polypeptides in such range amounts or comprising nucleic acids, plasmids, or expression vectors that can express such amounts in the subject are also contemplated.

In cancer immunotherapy or prophylactic applications (e.g., multiple mycloma, breast cancer, lymphoma, and the like), it is advantageous to administer at least one nucleic acid or vector of the invention in combination with at least one nucleic acid encoding a co-stimulatory molecule (including, e.g., B7-1, B7-1 variant, CD28 binding protein (e.g., CD28BP-15 as described in commonly assigned PCT application Ser. No. 01/19,973, published with International Publication No. WO 02/00717, filed Jun. 22, 2001, entitled “Novel Co-Stimulatory Molecules” (see nucleic acid sequence SEQ ID NO:19, and corresponding protein sequence SEQ ID NO:66, as set forth in WO 02/00717), and immune-enhancing or immune-stimulating fragments thereof, such as the polypeptide sequence comprising the extracellular domain of CD28BP-15 (SEQ ID NO:66) as described in PCT Appn. WO 02/00717, which application is incorporated herein by reference in its entirety for all purposes) and at least one cancer antigen (e.g., an antigen that induces antibodies against human EpCAM, an EpCAM mammalian variant, or human EpCAM or other cancer antigen such as described above), and if, desired with at least one other molecule of interest, such as,-e.g., a cytokine (IL-12, IL-15, IL-2, or variant thereof; etc.) and/or colony stimulating factor (e.g., GM-CSF). Such combination can serve to enhance a desired response, e.g., to enhance lymphocyte proliferation and/or gamma-interferon release. Included are recombinant, variant and mutant forms of IL-12, including recombinant IL-12p-40 and IL-12p35 polypeptides and nucleic acids described in PCT application Ser. No. 00/32,664 (International Publ. No. WO 01/40257), which application is incorporated herein by reference in its entirety for all purposes. In one format, a bicistronic vector of the invention comprising nucleotide sequences encoding an exogenous co-stimulatory polypeptide, exogenous cancer antigen, and other exogenous polypeptide(s) of interest is administered to the subject (e.g., by intramuscular or intradermal injection). In another format, a vector comprising a nucleotide sequence encoding the molecule of interest can be administered separately to the patient, at the same time or following administration of the one or more vectors comprising sequences encoding the antigen and/or additional exogenous polypeptide (such as a CD28 binding protein). Typically, a dose of at least about 1 mg nucleic acid (e.g., DNA) of GM-CSF and/or IL-2, IL-12 or other cytokine is administered at the time of immunization with the antigen-encoding and co-stimulatory-encoding nucleic acids. Alternatively, the additional molecule of interest (GM-CSF, IL-12, IL-2, or other cytokine) is administered to the subject as a polypeptide (e.g., by i.m. or i.d. injection). The initial dose of this polypeptide is administered at about the same time as the vector encoding the exogenous co-stimulatory polypeptide and antigen, and typically comprises at least about 75 ug. Subsequent additional “boost” doses of at least about 75 ug are usually delivered once/day for at least four days following the initial immunization. In another format, one or more vectors encoding either or both an exogenous polypeptide of interest (co-stimulatory molecule, cytokine, GM-CSF) are administered (via, e.g., i.d. or i.m. injection) in vivo into the tumor of a subject where the tumor is inoperable, or into tumor cells removed from a patient (ex vivo administration). Additional vector formats can also be used (adenoviral, retroviral, bicistronic, tricistronic). The toxicity and therapeutic efficacy of the vectors that include recombinant molecules provided by the invention are determined using standard pharmaceutical procedures in cell cultures or experimental animals. One can determine the LD₅₀ (the dose lethal to 50% of the population) and the ED₅₀ (the dose therapeutically effective in 50% of the population) using procedures presented herein and those otherwise known to those of skill in the art. Nucleic acids, polypeptides, proteins, fusion proteins, transduced cells and other formulations of the present invention can be administered at a rate determined, e.g., by the LD₅₀ of the formulation, and the side-effects thereof at various concentrations, as applied to the mass and overall health of the patient. Again, administration can be accomplished via single or divided doses.

A typical pharmaceutical composition for intravenous administration is about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. For recombinant promoters of the invention that express the linked transgene at high levels, it may be possible to achieve the desired effect using lower doses, e.g., on the order of about 1 μg or 10 μg per patient per day. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

For introduction of transduced cells comprising a nucleic acid vector of the invention (which comprises, e.g., an exogenous nucleic acid encoding at least one exogenous antigen, co-stimulatory molecule, cytokine, and/or adjuvant, or the like) into a patient, an illustrative, but not limiting, example includes taking blood samples, obtained prior to infusion, and saved for analysis. Between, e.g., 1×10⁶ and 1×10¹² transduced cells are infused intravenously over, e.g., 60-200 minutes. Vital signs and oxygen saturation by pulse oximetry are closely monitored. Blood samples are obtained, e.g., 5 minutes and, e.g., 1 hour following infusion and saved for subsequent analysis. Leukopheresis, transduction and reinfusion are optionally repeated every, e.g., 2 to 3 months for a total of, e.g., 4 to 6 treatments in a one year period. After the first treatment, infusions can be performed, e.g., on a outpatient basis at the discretion of the clinician. If the reinfusion is given as an outpatient, the participant is monitored for, e.g., at least 4, and preferably, e.g., 8 hours following the therapy. Transduced cells are prepared for reinfusion according to established methods. See, Abrahamsen et al. (1991) J Clin Apheresis 6:48-53; Carter et al. (1988) J Clin Arpheresis 4:113-117; Aebersold et al. (1988), J Immunol Methods 112:1-7; Muul et al. (1987) J Immunol Methods 101:171-181 and Carter et al. (1987) Transfusion 27:362-365. After a period of, e.g., about 2-4 weeks in culture, the cells should number between, e.g., 1×10⁶ and 1×10¹². In this regard, the growth characteristics of cells vary from patient to patient and from cell type to cell type. About, e.g., 72 hours prior to reinfusion of the transduced cells, an aliquot is taken for analysis of phenotype, and percentage of cells expressing the therapeutic agent.

If a patient undergoing infusion of a vector or transduced cell or protein formulation develops, e.g., fevers, chills, or muscle aches, he/she receives the appropriate dose of, e.g., aspirin, ibuprofen, acetaminophen or other pain/fever controlling drug. Patients who experience reactions to the infusion such as fever, muscle aches, and chills are premedicated, e.g., 30 minutes prior to the future infusions with, e.g., either aspirin, acetaminophen, or, e.g., diphenhydramine, etc. Meperidine is used for more severe chills and muscle aches that do not quickly respond to antipyretics and antihistamines. Cell infusion is, e.g., slowed or discontinued depending upon the severity of the reaction.

The nucleic acids, vectors, expression vectors, cells, transgenic animals, and compositions that include the nucleic acids of the invention (or the polypeptides encoded by any such nucleic acids or vectors) can be packaged in packs, dispenser devices, and kits for administration to a subject, such as a mammal. For example, packs or dispenser devices that contain one or more unit dosage forms are provided. Typically, instructions for administration of the compounds will be provided with the packaging, along with a suitable indication on the label that the compound is suitable for treatment of an indicated condition. For example, the label may state that the active compound within the packaging is useful for treating a particular infectious disease, autoimmune disorder, tumor, or for preventing or treating other diseases or conditions that are mediated by, or potentially susceptible to, a subject's or mammalian immune response.

Any nucleic acid, vector, plasmid, or cell of the invention described herein, and any composition comprising at least one such nucleic acid, vector, plasmid, or cell can be used in any of the methods and applications described herein. In one aspect, the invention provides for the use of any nucleic acid or vector (or cell comprising such nucleic acid or vector) or composition thereof as a medicament or vaccine, when administered in conjunction with an exogenous nucleic acid encoding a therapeutic or prophylactic polypeptide, antigen, co-stimulatory molecule, etc. for the treatment of one of the diseases described herein or for preventing one of the diseases described herein, or the like. In another aspect, the invention provides for the use of any nucleic acid or vector or cell comprising, either or composition thereof for the manufacture of a medicament, prophylactic, therapeutic, drug, or vaccine, including for any therapeutic or prophylactic application relating to treatment of a disease or disorder as described herein.

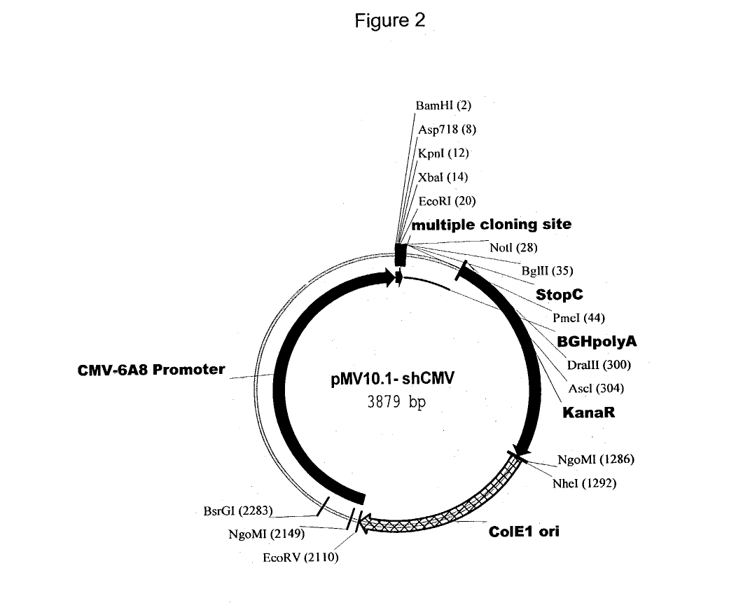

In one aspect, the invention provides methods for modulating or altering an immune response T-cell response specific to an antigen in a subject. Some such methods comprise administering to the subject at least one nucleic acid vector of the invention (e.g., SEQ ID NO:1 (FIG. 1) or SEQ ID NO:2 (FIG. 2)) that further comprises at least one exogenous polynucleotide encoding at least one exogenous co-stimulatory polypeptide (e.g., CD28 binding protein that enhances T cell activation and/or proliferation) (e.g., SEQ ID NO:3 (FIG. 3)) or a fragment thereof, and a polynucleotide sequence encoding the antigen or antigenic fragment thereof. For example, FIG. 4 shows an exemplary bicistronic expression vector that comprises two promoters, two polyA nucleotide sequences, and two transgenes, each of which transgene is operably linked to one of the promoters. The promoters can be the same or different. Similarly, the polyA nucleotide sequences can the same or different. In one embodiment, as specifically shown in FIG. 4, the two transgenes comprise two exogenous polynucleotide sequences encoding respective polypeptides of interest (e.g., a CD28BP-15 polypeptide and the cancer antigen, EpCAM) which are incorporated into the expression vector at the cloning sites using a variety of polylinkers suitable for cloning using the methods of vector construction described herein and those known by persons of ordinary skill in the art. Alternatively, a chimeric or shuffled cancer antigen can be used in place of the EpCAM/KSA antigen, including, e.g., a tumor-associated (TAg) as described in commonly assigned U.S. Provisional Patent Application Serial No. ______, entitled “Novel Tumor-Associated Antigens,” filed as Maxygen Attorney Docket No. 0334.11US on Apr. 22, 2003. Any antigen of interest can be incorporated into the vector in place of the EpCAM antigen shown in FIG. 4. For example, a viral antigen, such as a dengue virus antigen or shuffled or chimeric dengue virus antigen, parasitic antigen (e.g., malarial antigen), can be used.

For example, a polynucleotide sequence that encodes one or more viral antigens can be employed with nucleic acids and vectors of the invention. Such antigen-encoding polynucleotide sequence can be incorporated into the vector or nucleic acid sequence. Such antigens include, but are not limited to, influenza A virus N2 neuraminidase (Kilbourne et al. (1995) Vaccine 13: 1799-1803); Dengue virus envelope (E) and premembrane (prM) antigens (Feighny et al. (1994) Am. J Trop. Med. Hyg. 50: 322-328; Putnak et al. (1996) Am. J Trop. Med. Hyg. 55: 504-10); HIV antigens Gag, Pol, Vif and Nef (Vogt et al. (1995) Vaccine 13: 202-208); HIV antigens gp120 and gp160 (Achour et al. (1995) Cell. Mol. Biol. 41: 395-400; Hone et al. (1994) Dev. Biol. Stand. 82: 159-162); gp41 epitope of human immunodeficiency virus (Eckhart et al. (1996) J. Gen. Virol. 77: 2001-2008); rotavirus antigen VP4 (Mattion et al. (1995) J. Virol. 69: 5132-5137); the rotavirus protein VP7 or VP7sc (Emslie et al. (1995) J. Virol. 69: 1747-1754; Xu et al. (1995) J. Gen. Virol. 76: 1971-1980); herpes simplex virus (HSV) glycoproteins gB, gC, gD, gE, gG, gH, and gI (Fleck et al. (1994) Med. Microbiol. Immunol. (Berl) 183: 87-94 [Mattion, 1995]; Ghiasi et al. (1995) Invest. Ophthalmol. Vis. Sci. 36: 1352-1360; McLean et al. (1994) J. Infect. Dis. 170: 1100-1109); immediate-early protein ICP47 of herpes simplex virus-type 1 (HSV-1) (Banks et al. (1994) Virology 200: 236-245); immediate-early (IE) proteins ICP27, ICP0, and ICP4 of herpes simplex virus (Manickan et al. (1995) J. Virol. 69: 4711-4716); influenza virus nucleoprotein and hemagglutinin (Deck et al. (1997) Vaccine 15: 71-78; Fu et al. (1997) J. Virol. 71: 2715-2721); B19 parvovirus capsid proteins VP1 (Kawase et al. (1995) Virology 211: 359-366) or VP2 (Brown et al. (1994) Virology 198: 477-488); Hepatitis B virus core and e antigen (Schodel et al. (1996) Intervirology 39: 104-106); hepatitis B surface antigen (Shiau and Murray (1997) J. Med. Virol. 51: 159-166); hepatitis B surface antigen fused to the core antigen of the virus (Id.); Hepatitis B virus core-preS2 particles (Nemeckova et al. (1996) Acta Virol. 40: 273-279); HBV preS2-S protein (Kutinova et al. (1996) Vaccine 14: 1045-1052); VZV glycoprotein I (Kutinova et al. (1996) Vaccine 14: 1045-1052); rabies virus glycoproteins (Xiang et al. (1994) Virology 199: 132-140; Xuan et al. (1995) Virus Res. 36: 151-161) or ribonucleocapsid (Hooper et al. (1994) Proc. Nat'l. Acad. Sci. USA 91: 10908-10912); human cytomegalovirus (HCMV) glycoprotein B (UL55) (Britt et al. (1995) J. Infect. Dis. 171: 18-25); the hepatitis C virus (HCV) nucleocapsid protein in a secreted or a nonsecreted form, or as a fusion protein with the middle (pre-S2 and S) or major (S) surface antigens of hepatitis B virus (HBV) (Inchauspe et al. (1997) DNA Cell Biol. 16: 185-195; Major et al. (1995) J. Virol. 69: 5798-5805); the hepatitis C virus antigens: the core protein (pC); E1 (pE1) and E2 (pE2) alone or as fusion proteins (Saito et al. (1997) Gastroenterology 112: 1321-1330); the gene encoding respiratory syncytial virus fusion protein (PFP-2) (Falsey and Walsh (1996) Vaccine 14: 1214-1218; Piedra et al. (1996) Pediatr. Infect. Dis. J. 15: 23-31); the VP6 and VP7 genes of rotaviruses (Choi et al. (1997) Virology 232: 129-138; Jin et al. (1996) Arch. Virol. 141: 2057-2076); the E1, E2, E3, E4, E5, E6 and E7 protein of human papillomavirus (Brown et al. (1994) Virology 201: 46-54; Dillner et al. (1995) Cancer Detect. Prev. 19: 381-393; Krul et al. (1996) Cancer Immunol. Immunother. 43: 44-48; Nakagawa et al. (1997) J. Infect. Dis. 175: 927-931); a human T-lymphotropic virus type I gag protein (Porter et al. (1995) J. Med. Virol. 45: 469-474); Epstein-Barr virus (EBV) gp340 (Mackett et al. (1996) J. Med. Virol. 50: 263-271); the Epstein-Barr virus (EBV) latent membrane protein LMP2 (Lee et al. (1996) Eur. J. Immunol. 26: 1875-1883); Epstein-Barr virus nuclear antigens 1 and 2 (Chen and Cooper (1996) J. Virol. 70: 4849-4853; Khanna et al. (1995) Virology 214: 633-637); the measles virus nucleoprotein (N) (Fooks et al. (1995) Virology 210: 456-465); and cytomegalovirus glycoprotein gB (Marshall et al. (1994) J. Med. Virol. 43: 77-83) or glycoprotein gH (Rasmussen et al. (1994) J. Infect. Dis. 170: 673-677); an antigen of Japanese encephalitis virus; an antigen of arthropod-borne, encephalitic alphaviruses Venezuelan (VEEV), eastern (EEEV), and Western (WEEV) equine encephalitis viruses; or a variant, chimeric polypeptide, or derivative of any such viral antigen described herein.

Nucleotide sequences encoding one or more antigens from parasites can also be incorporated into a nucleic acid or vector of the invention. These include, but are not limited to, the schistosome gut-associated antigens CAA (circulating anodic antigen) and CCA (circulating cathodic antigen) in Schistosoma mansoni, S. haematobium or S. japonicum (Deelder et al. (1996) Parasitology 112: 21-35); a multiple antigen peptide (MAP) composed of two distinct protective antigens derived from the parasite Schistosoma mansoni (Ferru et al. (1997) Parasite Immunol. 19: 1-11); Leishmania parasite surface molecules (Lezama-Davila (1997) Arch. Med. Res. 28: 47-53); third-stage larval (L3) antigens of L. loa (Akue et al. (1997) J. Infect. Dis. 175: 158-63); the genes, Tams1-1 and Tams1-2, encoding the 30-and 32-kDa major merozoite surface antigens of Theileria annulata (Ta) (d'Oliveira et al. (1996) Gene 172: 33-39); Plasmodium falciparum merozoite surface antigen 1 or 2 (al-Yaman et al. (1995) Trans. R. Soc. Trop. Med. Hyg. 89: 555-559; Beck et al. (1997) J. Infect. Dis. 175: 921-926; Rzepczyk et al. (1997) Infect. Immun. 65: 1098-1100); circumsporozoite (CS) protein-based B-epitopes from Plasmodium berghei, (PPPPNPND)2 and Plasmodium yoelii, (QGPGAP)3QG, along with a P. berghei T-helper epitope KQIRDSITEEWS (Reed et al. (1997) Vaccine 15: 482-488); NYVAC-Pf7 encoded Plasmodium falciparum antigens derived from the sporozoite (circumsporozoite protein and sporozoite surface protein 2), liver (liver stage antigen 1), blood (merozoite surface protein 1, serine repeat antigen, and apical membrane antigen 1), and sexual (25-kDa sexual-stage antigen) stages of the parasite life cycle were inserted into a single NYVAC genome to generate NYVAC-Pf7 (Tine et al. (1996) Infect. Immun. 64: 3833-3844); Plasmodium falciparum antigen Pfs230 (Williamson et al. (1996) Mol. Biochem. Parasitol. 78: 161-169); Plasmodium falciparum apical membrane antigen (AMA-1) (Lal et al. (1996) Infect. Immun. 64: 1054-1059); Plasmodium falciparum proteins Pfs28 and Pfs25 (Duffy and Kaslow (1997) Infect. Immun. 65: 1109-1113); Plasmodium falciparum merozoite surface protein, MSP1 (Hui et al. (1996) Infect. Immun. 64: 1502-1509); the malaria antigen Pf332 (Ahlborg et al. (1996) Immunology 88: 630-635); Plasmodium falciparum erythrocyte membrane protein 1 (Baruch et al. (1995) Proc. Nat'l. Acad. Sci. USA 93: 3497-3502; Baruch et al. (1995) Cell 82: 77-87) and antigenic fragments thereof (see, e.g., WO 96/33736); Plasmodium falciparum merozoite surface antigen, PfMSP-1 (Egan et al. (1996) J. Infect. Dis. 173: 765-769); Plasmodium falciparum antigens SERA, EBA-175, RAP1 and RAP2 (Riley (1997) J. Pharm. Pharmacol. 49: 21-27); Schistosoma japonicum paramyosin (Sj97) or fragments thereof (Yang et al. (1995) Biochem. Biophys. Res. Commun. 212: 1029-1039); and Hsp70 in parasites (Maresca and Kobayashi (1994) Experientia 50: 1067-1074); or a variant, chimeric, or derivative of any such antigen described herein.

A nucleotide sequence encoding an allergen antigen can also included in a nucleic acid or vector of the invention. Examples of allergies that can be treated using a vector of the invention include, but are not limited to, allergies against house dust mite, grass pollen, birch pollen, ragweed pollen, hazel pollen, cockroach, rice, olive tree pollen, fungi, mustard, bee venom. Antigens of interest include those of animals, including the mite (e.g., Dermatophagoides pteronyssinus, Dermatophagoides farinae, Blomia tropicalis), such as the allergens der p1 (Scobie et al. (1994) Biochem. Soc. Trans. 22: 448S; Yssel et al. (1992) J. Immunol. 148: 738-745), der p2 (Chua et al. (1996) Clin. Exp. Allergy 26: 829-837), der p3 (Smith and Thomas (1996) Clin. Exp. Allergy 26: 571-579), der p5, der p V (Lin et al. (1994) J. Allergy Clin. Immunol. 94: 989-996), der p6 (Bennett and Thomas (1996) Clin. Exp. Allergy 26: 1150-1154), der p 7 (Shen et al. (1995) Clin. Exp. Allergy 25: 416-422), der f2 (Yuuki et al. (1997) Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Mag 3 (Fujikawa et al. (1996) Mol. Immunol. 33: 311-319). Also of interest as antigens are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 Da, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ c1 (Gregoire et al. (1996) J. Biol. Chem. 271: 32951-32959); Jumper ant M. pilosula allergen Myr p I and its homologous allergenic polypeptides Myr p2 (Donovan et al. (1996) Biochem. Mol. Biol. Int. 39: 877-885); 1-13, 14, 16 kD allergens of the mite Blomia tropicalis (Caraballo et al. (1996) J. Allergy Clin. Immunol. 98: 573-579); the cockroach allergens Bla g Bd90K (Helm et al. (1996) J. Allergy Clin. Immunol. 98: 172-80) and Bla g 2 (Arruda et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f1 or cat allergen Fel d 1 (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kD, 25 kD or 60 kD (Spitzauer et al. (1997) Clin. Exp. Allergy 27: 196-200); and the 20 kd major allergen of cow (Ylonen et al. (1994) J. Allergy Clin. Immunol. 93: 851-858).

Pollen and grass allergens are also useful in vaccines, particularly after optimization of the antigen by the methods of the invention. Such allergens include, for example, Hor v⁹ (Astwood and Hill (1996) Gene 182: 53-62, Lig v1 (Batanero et al. (1996) Clin. Exp. Allergy 26: 1401-1410); Lol p 1 (Muller et al. (1996) Int. Arch. Allergy Immunol. 109: 352-355), Lol p II (Tamborini et al. (1995) Mol. Immunol. 32: 505-513), Lol pVA, Lol pVB (Ong et al. (1995) Mol. Immunol. 32: 295-302), Lol p 9 (Blaher et al. (1996) J. Allergy Clin. Immunol. 98: 124-132); Par J. I (Costa et al. (1994) FEBS Lett. 341: 182-186; Sallusto et al. (1996) J. Allergy Clin. Immunol. 97: 627-637), Parj 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Phl p 1 (Petersen et al. (1995) J. Allergy Clin. Immunol. 95: 987-994), Phl p 5 (Muller et al. (1996) Int. Arch. Allergy Immunol. 109: 352-355), Phl p 6 (Petersen et al. (1995) Int. Arch. Allergy Immunol. 108: 55-59); Cryj I (Sone et al. (1994) Biochem. Biophys. Res. Commun. 199: 619-625), Cry j II (Namba et al. (1994) FEBS Lett. 353: 124-128); Cora 1 (Schenk et al. (1994) Eur. J. Biochem. 224: 717-722); cyn d1 (Smith et al. (1996) J. Allergy Clin. Immunol. 98: 331-343), cyn d7 (Suphioglu et al. (1997) FEBS Lett. 402: 167-172); Pha a 1 and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, e.g, from timothy grass or birch pollen (Valenta et al. (1994) Biochem. Biophys. Res. Commun. 199: 106-118); P0149 (Wu et al. (1996) Plant Mol. Biol. 32: 1037-1042); Ory s1 (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073);or a variant, chimeric, or derivative of any such antigen.

Fungal allergens useful in these vectors and vaccines include, but are not limited to, the allergen, Cla h III, of Cladosporium herbarum (Zhang et al. (1995) J. Immunol. 154: 710-717); the allergen Psi c 2, a fungal cyclophilin, from the basidiomycete Psilocybe cubensis (Horner et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kD allergen of Penicillium notatum (Shen et al. (1995) Clin. Exp. Allergy 26:.350-356); aldehyde dehydrogenase (ALDH) (Achatz et al. (1995) Mol Immunol. 32: 213-227); enolase (Achatz et al. (1995) Mol. Immunol. 32: 213-227); YCP4 (Id.); acidic ribosomal protein P2 (Id.). Other allergens that can be used in the methods of the invention include latex allergens, such as a major allergen (Hev b 5) from natural rubber latex (Akasawa et al. (1996) J. Biol. Chem. 271: 25389-25393; Slater et al. (1996) J. Biol. Chem. 271: 25394-25399); ;or a variant, chimeric, or derivative of any such antigen.

Among the tumor-specific antigens that can be used in vectors, nucleic acids and methods of the invention are: bullous pemphigoid antigen 2, prostate mucin antigen (PMA) (Beckett and Wright (1995) Int. J. Cancer 62: 703-710), tumor associated Thomsen-Friedenreich antigen (Dahlenborg et al. (1997) Int. J. Cancer 70: 63-71), prostate-specific antigen (PSA) (Dannull and Belldegrun (1997) Br. J. Urol. 1: 97-103), luminal epithelial antigen (LEA.135) of breast carcinoma and bladder transitional cell carcinoma (TCC) (Jones et al. (1997) Anticancer Res. 17: 685-687), cancer-associated serum antigen (CASA) and cancer antigen 125 (CA 125) (Kierkegaard et al. (1995) Gynecol. Oncol. 59: 251-254), the epithelial glycoprotein 40 (EGP40) (Kievit et al. (1997) Int. J. Cancer 71: 237-245), squamous cell carcinoma antigen (SCC) (Lozza et al. (1997) Anticancer Res. 17: 525-529), cathepsin E (Mota et al. (1997) Am. J. Pathol. 150: 1223-1229), tyrosinase in melanoma (Fishman et al. (1997) Cancer 79: 1461-1464), cell nuclear antigen (PCNA) of cerebral cavemomas (Notelet et al. (1997) Surg. Neurol. 47: 364-370), DF3/MUC1 breast cancer antigen (Apostolopoulos et al. (1996) Immunol. Cell. Biol. 74: 457-464; Pandey et al. (1995) Cancer Res. 55: 4000-4003), carcinoembryonic antigen (Paone et al. (1996) J Cancer Res. Clin. Oncol. 122: 499-503; Schlom et al. (1996) Breast Cancer Res. Treat. 38: 27-39), tumor-associated antigen CA 19-9 (Tolliver and O'Brien (1997) South Med. J. 90: 89-90; Tsuruta et al. (1997) Urol. Int. 58: 20-24), human melanoma antigens MART-1/Melan-A27-35 and gp100 (Kawakami and Rosenberg (1997) Int. Rev. Immunol. 14:173-192; Zajac et al. (1997) Int. J. Cancer 71: 491-496), the T and Tn pancarcinoma (CA) glycopeptide epitopes (Springer (1995) Crit. Rev. Oncog. 6: 57-85), a 35 kD tumor-associated autoantigen in papillary thyroid carcinoma (Lucas et al. (1996) Anticancer Res. 16: 2493-2496), KH-1 adenocarcinoma antigen (Deshpande and Danishefsky (1997) Nature 387: 164-166), the A60 mycobacterial antigen (Maes et al. (1996) J. Cancer Res. Clin. Oncol. 122: 296-300), heat shock proteins (HSPs) (Blachere and Srivastava (1995) Semin. Cancer Biol. 6: 349-355), and MAGE, tyrosinase, melan-A and gp75 and mutant oncogene products (e.g., p53, ras, and HER-2/neu (Bueler and Mulligan (1996) Mol. Med. 2: 545-555; Lewis and Houghton (1995) Semin. Cancer Biol. 6: 321-327; Theobald et al. (1995) Proc. Nat'l. Acad. Sci. USA 92: 11993-11997).

Nucleic acids that encode autoantigens that can be incorporated in the vectors and methods of the invention and used in vaccines for treating multiple sclerosis include, but are not limited to, myelin basic protein (Stinissen et al. (1996) J. Neurosci. Res. 45: 500-511) or a fusion protein of myelin basic protein and proteolipid protein (Elliott et al. (1996) J. Clin. Invest. 98: 1602-1612), proteolipid protein (PLP) (Rosener et al. (1997) J. Neuroimmunol. 75: 28-34), 2′,3′-cyclic nucleotide 3′-phosphodiesterase (CNPase) (Rosener et al. (1997) J. Neuroimmunol. 75: 28-34), the Epstein Barr virus nuclear antigen-1 (EBNA-1) (Vaughan et al. (1996) J. Neuroimmunol. 69: 95-102), HSP70 (Salvetti et al. (1996) J. Neuroimmunol. 65: 143-53; Feldmann et al. (1996) Cell 85: 307).

The vectors, nucleic acids and methods of the invention are also useful for treating insulin dependent diabetes mellitus, using one or more of antigens which include, but are not limited to, insulin, proinsulin, GAD65 and GAD67, heat-shock protein 65 (hsp65), and islet-cell antigen 69 (ICA69) (French et al. (1997) Diabetes 46: 34-39; Roep (1996) Diabetes 45: 1147-1156; Schloot et al. (1997) Diabetologia 40: 332-338), viral proteins homologous to GAD65 (Jones and Crosby (1996) Diabetologia 39: 1318-1324), islet cell antigen-related protein-tyrosine phosphatase (PTP) (Cui et al. (1996) J. Biol. Chem. 271: 24817-24823), GM2-1 ganglioside (Cavallo et al. (1996) J. Endocrinol. 150: 113-120; Dotta et al. (1996) Diabetes 45: 1193-1196), glutamic acid decarboxylase (GAD) (Nepom (1995) Curr. Opin. Immunol. 7: 825-830; Panina-Bordignon et al. (1995) J. Exp. Med. 181: 1923-1927), an islet cell antigen (ICA69) (Karges et al. (1997) Biochim. Biophys. Acta 1360: 97-101; Roep et al. (1996) Eur. J. Immunol. 26: 1285-1289), Tep69, the single T cell epitope recognized by T cells from diabetes patients (Karges et al. (1997) Biochim. Biophys. Acta 1360: 97-101), ICA 512, an autoantigen of type I diabetes (Solimena et al. (1996) EMBO J. 15: 2102-2114), an islet-cell protein tyrosine phosphatase and the 37-kDa autoantigen derived from it in type 1 diabetes (including IA-2, IA-2) (La Gasse et al. (1997) Mol. Med. 3: 163-173), the 64 kDa protein from In-111 cells or human thyroid follicular cells that is immunoprecipitated with sera from patients with islet cell surface antibodies (ICSA) (Igawa et al. (1996) Endocr. J. 43: 299-306), phogrin, a homologue of the human transmembrane protein tyrosine phosphatase, an autoantigen of type 1 diabetes (Kawasaki et al. (1996) Biochem. Biophys. Res. Commun. 227: 440-447), the 40 kDa and 37 kDa tryptic fragments and their precursors IA-2 and IA-2 in IDDM (Lampasona et al. (1996) J. Immunol. 157: 2707-2711; Notkins et al. (1996) J. Autoimmun. 9: 677-682), insulin or a cholera toxoid-insulin conjugate (Bergerot et al. (1997) Proc. Nat'l. Acad. Sci. USA 94: 4610-4614), carboxypeptidase H, the human homologue of gp330, which is a renal epithelial glycoprotein involved in inducing Heymann nephritis in rats, and the 38-kD islet mitochondrial autoantigen (Arden et al. (1996) J. Clin. Invest. 97: 551-561.

Rheumatoid arthritis is another condition that is treatable using nucleic acids and vectors of the invention with antigens for rheumatoid arthritis. Useful antigens for rheumatoid arthritis treatment include, but are not limited to, the 45 kDa DEK nuclear antigen, in particular onset juvenile rheumatoid arthritis and iridocyclitis (Murray et al. (1997) J. Rheumatol. 24: 560-567), human cartilage glycoprotein-39, an autoantigen in rheumatoid arthritis (Verheijden et al. (1997) Arthritis Rheum. 40: 1115-1125), a 68 k autoantigen in rheumatoid arthritis (Blass et al. (1997) Ann. Rheum. Dis. 56: 317-322), collagen (Rosloniec et al. (1995) J. Immunol. 155: 4504-4511), collagen type II (Cook et al. (1996) Arthritis Rheum. 39: 1720-1727; Trentham (1996) Ann. N. Y. Acad. Sci. 778: 306-314), cartilage link protein (Guerassimov et al. (1997) J. Rheumatol. 24: 959-964), ezrin, radixin and moesin, which are auto-immune antigens in rheumatoid arthritis (Wagatsuma et al. (1996) Mol. Immunol. 33: 1171-1176), and mycobacterial heat shock protein 65 (Ragno et al. (1997) Arthritis Rheum. 40: 277-283).

Also among the conditions for which one can obtain an improved antigen suitable for treatment are autoimmune thyroid disorders. Antigens that are useful for these applications include, for example, thyroid peroxidase and the thyroid stimulating hormone receptor (Tandon and Weetman (1994) J. R. Coll. Physicians Lond. 28: 10-18), thyroid peroxidase from human Graves' thyroid tissue (Gardas et al. (1997) Biochem. Biophys. Res. Commun. 234: 366-370; Zimmer et al. (1997) Histochem. Cell. Biol. 107: 115-120), a 64-kDa antigen associated with thyroid-associated ophthalmopathy (Zhang et al. (1996) Clin. Immunol. Immunopathol. 80: 236-244), the human TSH receptor (Nicholson et al. (1996) J. Mol. Endocrinol. 16: 159-170), and the 64 kDa protein from In-111 cells or human thyroid follicular cells that is immunoprecipitated with sera from patients with islet cell surface antibodies (ICSA) (Igawa et al. (1996) Endocr. J. 43: 299-306).

Other conditions and associated antigens include, but are not limited to, Sjogren's syndrome (-fodrin; Haneji et al. (1997) Science 276: 604-607), myastenia gravis (the human M2 acetylcholine receptor or fragments thereof, specifically the second extracellular loop of the human M2 acetylcholine receptor; Fu et al. (1996) Clin. Immunol. Immunopathol. 78: 203-207), vitiligo (tyrosinase; Fishman et al. (1997) Cancer 79: 1461-1464), a 450 kD human epidermal autoantigen recognized by serum from individual with blistering skin disease, and ulcerative colitis (chromosomal proteins HMG1 and HMG2; Sobajima et al. (1997) Clin. Exp. Immunol. 107: 135-140).

Polynucleotide sequences that encode co-stimulatory and/or immunomodulatory molecules that can be incorporated into nucleic acids, vectors and methods of the invention include those described in WO 99/41368 by Punnonen et al. “Optimization of Immunomodulatory Properties of Genetic Vaccines,” which is incorporated herein by reference in its entirety for all purposes.

Different promoters can be used in the expression vector to effectuate selectively different expression of exogenous therapeutic or prophylactic polypeptides that are encoded by exogenous polynucleotide of interests operably linked to the promoters. For example, a “strongly-expressing” promoter can be operably linked to an exogenous polynucleotide for enhanced, stronger expression of the corresponding polypeptide, while a weaker or less strongly-expressing promoter can be operably linked to a second exogenous polynucleotide for less strong expression of the corresponding polypeptide. In the method described above for treating cancer, for example, the vector comprises a strongly expressing promoter is operably linked to a polynucleotide sequence encoding a cancer antigen, and a less strongly expressing promoter is operably linked to a second polynucleotide sequence encoding a co-stimulatory polypeptide that preferentially binds CD28 receptor (e.g., CD28BP-15 as described herein).

In some such methods, the antigen or antigenic fragment thereof is an antigen or antigenic fragment thereof of an infectious agent (e.g., hepatitis A, B, C, dengue virus, HIV) or a cancer (e.g., colon, breast, rectal, colorectal cancer).

As described above, a nucleic acid vector of the invention may further comprise one or more exogenous polynucleotide sequences, each of which encodes an exogenous polypeptide or fragment of interest (e.g., therapeutic or prophylactic polypeptide). Each such exogenous polynucleotide sequence may be operably linked to a promoter in the vector. In one embodiment, two or more exogenous polynucleotide sequences are included in the same vector (e.g., bicistronic vector); the first exogenous polynucleotide sequence is operably linked to a first promoter, and the second exogenous polynucleotide sequence is operably linked to a second promoter in the same vector. Alternatively, polynucleotide sequences encoding exogenous polypeptides of interest are administered in separate vectors and administered separately, e.g., either simultaneously or consecutively.

The nucleic acids and vectors of the invention may function as multicomponent vaccines by incorporating one or more exogenous nucleotide sequences that useful as components in multi-component vaccines. A multicomponent vaccine may optionally comprise, e.g., a single vector with multiple components or multiple vectors, each encoding different vector components or a multi-component protein-based vaccines in which a polypeptide of interest is delivered with other proteins (e.g., protein vaccine). The vectors encoding one or more polypeptides of interest (e.g., antigen or co-stimulatory polypeptide) can be delivered simultaneously or at different times, and optionally with other vector(s) or protein(s) if desired. Vectors of the invention can also be administered to a subject following delivery of a protein vaccine or DNA vaccine to boost the immune response to the protein vaccine or DNA vaccine. A multi-component vaccine optionally comprises, e.g., a vector, such as a DNA plasmid vector, that comprises, for example, in addition to nucleotide sequences encoding one or more co-stimulatory polypeptides, one or more nucleotide sequences encoding at least of the following components: at least one antigen(s), cytokine(s), adjuvant(s), promoter (e.g., wild-type CMV promoter (such as human CMV promoter with or without an intron A sequence; or a recombinant, or chimeric CMV promoter with or without a recombinant or WT intron A sequence), and/or other co-stimulatory molecule(s) (each of which may have been optimized by recursive sequence recombination and selection/screening procedures, random mutagenesis, or other known mutagenesis procedures), and combinations of such various components. Such multi-component vector expresses two or more such components and includes appropriate expression elements for such expression. Such an arrangement permits co-delivery of various components, including recursively-recombined components, for a particular treatment regimen or therapeutic or prophylactic application. Such vectors are designed according to the specific treatment regimen or therapeutic or prophylactic application desired. One or more such single-component or multi-component vectors as described above may be used simultaneously or in sequential administration in a therapeutic or prophylactic treatment method of the invention.

The nucleic acids and vectors of the invention are useful in treatment methods requiring administration to a subject an exogenous polypeptide of interest. The nucleic acids and vectors of the invention may further incorporate polynucleotides encoding such polypeptides of interest.

For example, nucleic acids and/or vectors of the invention may be constructed to further include a polynucleotide sequence encoding an antigen. Such nucleic acids and/or vectors are useful as DNA vaccines against diseases associated with such antigen(s) and/or in therapeutic and/or prophylactic methods for treating or preventing diseases associated with such antigen(s). For example, the incorporation of an exogenous polynucleotide sequence encoding a viral antigen, such as a dengue virus antigen, into a “backbone” pMaxVax10.1 expression vector of the invention (e.g., as shown in SEQ ID NO:1 (FIG. 1)) produces an expression vector useful as a DNA vaccine against dengue virus infection.

Nucleic acids and vectors of the invention can be used to express, deliver, and/or administer to a subject a variety of exogenous polypeptides of interest useful in therapeutic and prophylactic treatment of diseases and conditions, including, e.g., allergy/asthma, neurological, organ transplantation (e.g., graft versus host disease, and autoimmune diseases), malignant diseases, chronic infectious diseases, including, but not limited to, e.g., viral infectious diseases, such as those associated with, but not limited to, e.g., hepatitis B virus (HBV), herpes simplex virus (HSV), hepatitis C virus (HCV), HIV, human papilloma virus (HPV), and the like, and bacterial infectious diseases, such as, but not limited to, e.g., Lyme disease, tuberculosis, and chlamydia infections, and the like. A polynucleotide sequence encoding the appropriate exogenous polypeptide of interest can be incorporated into the nucleic acid or vector of the invention using standard cloning techniques and the methods described herein. Polylinkers within a nucleic acid or vector of the invention as described herein can be changed to incorporate additional or different restrictions sites to permit incorporation of specific exogenous polynucleotide sequences of interest. The polylinker is selected depending upon the polynucleotide of interest, and the polylinker can be readily changed or modified to accommodate a different polynucleotide sequence to be incorporated into the nucleic acid vector using standard techniques.

In one aspect, the invention provides an expression vector (e.g., SEQ ID NO:1 or 2) further comprising a polynucleotide sequence encoding a CTLA4BP polypeptide, or fragment thereof as described in commonly assigned WO 02/00717. CTLA-4BP polypeptides modulate T cell proliferation and/or activation and inhibit the immune response in autoimmune diseases or, as soluble molecules, act as antagonists.

For example, in one embodiment a polynucleotide sequence encoding the polypeptide of SEQ ID NO:86 as shown in WO 02/00717 (CTLA-4BP clone 5x4-12C) is incorporated into a pMaxVax10.1 expression vector (e.g., SEQ ID NO:1 or 2). One such polynucleotide encoding SEQ ID NO:86 shown in WO 02/00717 is SEQ ID NO:39 as set forth in WO 02/00717. Such a CTLA-4BP polypeptide can be delivered in a treatment protocol as a component of a DNA vaccine vector, as a full-length polypeptide, as a soluble polypeptide subsequence of the full-length CTLA-4BP polypeptide (e.g., ECD) used, if desired, as a polypeptide or protein vaccine or “boosting” polypeptide, or as a soluble fusion protein comprising a full-length CTLA-4BP polypeptide or subsequence thereof, such as a soluble polypeptide subsequence (e.g., ECD); in such formats, the CTLA-4BP polypeptide may serve as an agonist.

As discussed above, genetic vaccine comprising a vector comprising a nucleic acid sequence encoding a CTLA4-BP polypeptide (SEQ ID NO:86 as shown in WO 02/00717 (CTLA-4BP clone 5×4-12C)) and at least one nucleic acid sequence encoding at least one additional polypeptide of interest is also a feature of the invention. For example, in a DNA vaccine, in combination with a specific allergen, the CTLA4BPs (or fragments thereof, or soluble and/or fusion proteins thereof) may inhibit the allergen specific T cell response in allergy. Similarly, in combination with a specific auto-antigen, such as myclin basic protein, the CTLA-4BPs (or fragments thereof, or soluble and/or fusion proteins thereof) may inhibit the auto-antigen-specific T cell response in autoimmunity, such as in multiple sclerosis.

Examples of useful pathogen antigens, cancer antigens, allergens, and auto-antigens whose polynucleotide sequence can be incorporated into nucleic acids or vectors of the invention and used in methods of the invention have been provided in the following commonly assigned patent applications: Punnonen et al. (1999) WO 99/41369; Punnonen et al. (1999) WO 99/41383; Punnonen et al. (1999) WO 99/41368; and Punnonen et al. (1999) WO 99/41402), each of which is incorporated herein by reference in its entirety for all purposes. Several other useful antigens have been described in the literature or can be discovered using genomics approaches. Since typical tumor antigens are self proteins and thus host tolerant, it is optionally necessary to generate “non-self” tumor antigens that induce cross-reactivity against self tumor antigens also. This is optionally accomplished through, e.g., recursive sequence recombination of existing tumor antigens from diverse species to produce chimeric tumor antigens. Such chimeric antigens are then screened for ones that activate antigen-specific T cells which also recognize the wild-type tumor antigen. Optional screenings test whether chimeric antigens activate patient T cells (e.g., T cell lines specific for wild-type antigens generated and activation induced by APCs expressing recursively recombined antigens analyzed) and whether the chimeric antigen induces T cells that recognize wild-type antigen (e.g., T cell lines specific for recursively recombined antigens generated and activation induced by APCs expressing WT antigen analyzed).

Examples of useful antigens whose polynucleotide sequences can be incorporated into nucleic acids or vectors of the invention and used in methods of the invention are provided in Punnonen et al. (1999) WO 99/41383, which is incorporated herein by reference in its entirety for all purposes.

In one embodiment, the invention provides an expression vector (e.g., SEQ ID NO:3), which comprises an exogenous CD28BP-encoding polynucleotide sequence or CTLA-4BP-encoding polynucleotide sequence. CD28BP-encoding polynucleotide sequences and CTLA-4BP polynucleotide sequences are set forth in WO 02/00717. Such vector is useful in therapeutic or prophylactic treatment methods for treating or preventing any of the above-mentioned diseases and disorders when administered to a subject as a polypeptide (e.g. administer at least one full-length or soluble CD28BP polypeptide or fragment thereof) or cell-based vaccine (e.g., cell expressing or secreting at least one CD28BP polypeptide) or a gene-based therapeutic polypeptide (i.e., polypeptide product expressed by a CD28BP encoding polynucleotide), wherein such CD28BP polypeptides are delivered alone or co-administered simultaneously or subsequently with one or more of an antigen, another co-stimulatory molecule, or adjuvant. A CD28BP polypeptide is useful for treating or preventing any of the above-mentioned diseases and disorders when administered to a subject as a genetic vaccine (e.g., DNA vaccine) in which at least one CD28BP-encoding polynucleotide (e.g., SEQ ID NO:19 in WO 02/0717 or an extracellular domain-encoding polynucleotide fragment thereof) is administered alone or in a plasmid vector or gene therapy format (i.e., a vector encoding at least one CD28BP or CTLA-4 polypeptide). Or, if desired, at least one CD28BP-encoding or CTLA-4BP-encoding polynucleotide is co-administered with a second DNA vector encoding at least one of an antigen, co-stimulatory molecule, and/or adjuvant. Alternatively, if desired, a vector comprising at least one CD28BP-encoding or CTLA-4BP-encoding polynucleotide sequence and at least one of an antigen, allergen, co-stimulatory polypeptide, and/or adjuvant can be prepared and administered to a subject in a treatment protocol; in this instance, the at least one CD28BP-encoding or CTLA-4BP-encoding polynucleotide is co-expressed with at least one antigen, co-stimulatory molecule, allergen and/or adjuvant.

Additional examples of cancer antigens whose polynucleotide sequences can be incorporated into nucleic acids or vectors of the invention for expression, administration, and/or delivery of such antigens to a subject and used in methods of the invention described herein include, e.g., EpCAM/KSA, bullous pemphigoid antigen 2, prostate mucin antigen (PMA) (Beckett and Wright (1995) Int. J. Cancer 62:703-710), tumor associated Thomsen-Friedenreich antigen (Dahlenborg et al. (1997) Int. J. Cancer 70:63-71), prostate-specific antigen (PSA) (Dannull and Belldegrun (1997) Br. J. Urol. 1:97-103), luminal epithelial antigen (LEA.135) of breast carcinoma and bladder transitional cell carcinoma (TCC) (Jones et al. (1997) Anticancer Res. 17:685-687), cancer-associated serum antigen (CASA) and cancer antigen 125 (CA 125) (Kierkegaard et al. (1995) Gynecol. Oncol. 59:251-254), the epithelial glycoprotein 40 (EGP40) (Kievit et al. (1997) Intl. J. Cancer 71:237-245), squamous cell carcinoma antigen (SCC) (Lozza et al. (1997) Anticancer Res. 17: 525-529), cathepsin E (Mota et al. (1997) Am. J. Pathol. 150:1223-1229), tyrosinase in melanoma (Fishman et al. (1997) Cancer 79: 1461-1464), cell nuclear antigen (PCNA) of cerebral cavemomas (Notelet et al. (1997) Sure. Neurol. 47: 364-370), DF3/MUC1 breast cancer antigen (Apostolopoulos et al. (1996) Immunol. Cell. Biol. 74: 457-464; Pandey et al. (1995) Cancer Res. 55: 4000-4003), carcinoembryonic antigen (Paone et al. (1996) J. Cancer Res. Clin. Oncol. 122:499-503; Schlom et al. (1996) Breast Cancer Res. Treat. 38:27-39), tumor-associated antigen CA 19-9 (Tolliver and O'Brien (1997) South Med. J. 90:89-90; Tsuruta et al. (1997) Urol. Intl. 58:20-24), human melanoma antigens MART-1/Melan-A27-35 and gp100 (Kawakami and Rosenberg (1997) Intl. Rev. Immunol. 14:173-192; Zajac et al. (1997) Intl. J. Cancer 71:491-496), the T and Tn pancarcinoma (CA) glycopeptide epitopes (Springer (1995) Crit. Rev. Oncog. 6:57-85), a 35 kD tumor-associated autoantigen in papillary thyroid carcinoma (Lucas et al. (1996) Anticancer Res. 16:2493-2496), KH-1 adenocarcinoma antigen (Deshpande and Danishefsky (1997) Nature 387:164-166), the A60 mycobacterial antigen (Maes et al. (1996) J. Cancer Res. Clin. Oncol. 122:296-300), heat shock proteins (HSPs) (Blachere and Srivastava (1995) Semin. Cancer Biol. 6:349-355), and MAGE, tyrosinase, melan-A and gp75 and mutant oncogene products (e.g., p53, ras, CDk4, and HER-2/neu (Bueler and Mulligan (1996) Mol. Med. 2:545-555; Lewis and Houghton (1995) Semin. Cancer Biol. 6: 321-327; Theobald et al. (1995) Proc. Nat'l. Acad. Sci. USA 92: 11993-11997), prostate specific membrane antigen (PSMA) Bangma C H et al. (2000) Microsc Res Tech 51:430-5, TAG-72, McGuinness R P et al. Hum Gene Ther (1999) 10:165-73, and variants, derivatives, and mutated, and recombinant forms (e.g., shuffled forms) thereof of these antigens. Cancers that can be treated by using nucleic acids and vectors of the invention that further comprise one or more polynucleotide sequences encoding one or more cancer antigens include, but are not limited to, e.g., colorectal cancer, breast cancer, pancreatic cancer, lung cancer, prostate cancer, naso-pharyngeal cancer, cancer, brain cancer, leukemia, melanoma, head- and neck cancer, stomach cancer, cervical cancer, ovarian cancer, and lymphomas.

The invention also provides for gene therapy vectors (e.g., adenovirus (AV), adeno-associated virus (AAV), retrovirus, poxvirus, or lentivirus vectors) comprising at least one nucleic acid sequence of the invention or fragment thereof, optionally including an exogenous polynucleotide encoding a therapeutic or prophylactic polypeptide of interest.

Kits

The present invention also provides kits including one or more of the nucleic acids, vectors, expression vectors, cells, vaccines, polypeptides, and compositions of the invention. Kits of the invention optionally comprise at least one of the following of the invention: (1) at least one kit component comprising a nucleic acid, polynucleotide vector, or fragment thereof; plasmid expression vector; cell comprising a nucleic acid or vector or fragment thereof; and/or a composition or vaccine composition comprising at least one of any such component; (2) instructions for practicing any method described herein, including a therapeutic or prophylactic methods, instructions for using any component identified in (1) or any vaccine or composition of any such component; and/or instructions for operating any apparatus, system or component described herein; (3) optionally a container for holding said at least one such component or composition, and (4) optionally-packaging materials.

In a further aspect, the invention provides for the use of any component, composition, or kit described herein, for the practice of any method described herein, and/or for the use of any component, composition, or kit to practice any method described herein.

### EXAMPLES

The following examples are offered to illustrate the present invention, but not to limit the spirit or scope of the present invention in any way.

### Example 1

Construction of a Nucleic Acid Vector

This example describes the construction of an exemplary mammalian vector for expression in mammalian cells; in some embodiments, the vector is termed “pMaxVax10.1.” The mammalian expression vector pMaxVax10.1 comprises, among other things: (1) a promoter for driving the expression of a transgene or other nucleotide sequence in mammalian cells (including, e.g., but not limited to, a CMV promoter or a variant thereof, and shuffled, synthetic, or recombinant promoters, including those described in PCT Appn. No. WO 02/00897; (2) a polylinker for cloning of one or more additional nucleotide sequences (e.g., exogenous sequences, such as an exogenous sequence encoding an antigen, co-stimulatory molecule, adjuvant, a transgene coding sequence, etc.); (3) a polyadenylation signal (polyA); and (4) a prokaryotic replication origin and antibiotic resistant gene for amplification in E. coli. The construction of the vector is briefly described herein, although several suitable alternative techniques are available to produce such a DNA vector (e.g., applying the principles described elsewhere herein).

In one aspect, the pMaxVax10.1 vector comprises the polynucleotide sequence set forth in SEQ ID NO:1. In another aspect, the pMaxVax10.1 vector comprises the polynucleotide sequence set forth in SEQ ID NO:2. Exemplary embodiments of expression vectors of the invention are shown, e.g., in FIGS. 1 and 2.

In one embodiment, the minimal plasmid Col/Kana comprises the replication origin ColE1 and the kanamycin resistance gene (Kana^{r}). The ColE1 origin of replication (ori) mediates high copy number plasmid amplification.

In one embodiment, the ColE1 ori was isolated from vector pUC19 (New England Biolabs, Inc.) by application of standard PCR techniques. To link the ColE1 origin to the Kana^{r} gene, NgoMIV (or “NgoMI”) and DraIII recognition sequences were added to the 5′ and 3′ PCR primers, respectively. NgoMIV and DraIII are unique cloning sites in pMaxVax10.1. For subsequent cloning of the mammalian transcription unit, the 5′ forward primer also was designed to include the additional restriction site NheI downstream of the NgoMIV site and EcoRV and BsrGI cloning sites upstream of the DraIII site the 3′ reverse primer. All of the primers were designed to include additional 6-8 base pairs overhang for optimal restriction digest. Specifically, the sequence for the 5′ forward primer (“pMaxVax primer 1”) is acacatagcgccggcgctagctgagcaaaaggccagcaaaaggcca (SEQ ID NO:6) and the sequence for the 3′ reverse primer (“pMaxVax primer 2”) aactctgtgagacaacagtcataaatgtacagatatcagaccaagtttactcatatatac (SEQ ID NO:7).

Typically, the ColE1 PCR reactions were performed with proof-reading polymerases, such as Tth (PE Applied Biosystems), Pfu, PfuTurbo and Herculase (Stratagene), or Pwo (Roche), under conditions in accordance with the manufacturer's recommendations. By way of illustration, a typical Herculase polymerase PCR reaction contains 1 μl template plasmid DNA (1-10 ng/μl), 5 μl 10× buffer, 1 μl dNTPs (deoxynucleotide triphosphates) at 10 mM each, 1 μl forward primer (20 μM), 1 μl reverse primer (20 μM), 40 μl deionized, sterile water and 0.5 μl Herculase polymerase in a 50 μl reaction. Such PCR reactions were performed at 94° C. for 30 seconds, 55° C. for 30 seconds, and 72° C. for 30 seconds per cycle, for a total of 25 cycles.

The ColE1 PCR product was purified with phenol/chloroform using Phase lock Gel™Tube (Eppendorf) followed by standard ethanol precipitation. The purified ColE1 PCR product was digested with the restriction enzymes NgoMIV and DraIII according to the manufacturer's recommendations (New England Biolabs, Inc.) and gel purified using the QiaExII gel extraction kit (Qiagen) according to the manufacturer's instructions.

In this embodiment, the Kanamnycin resistance gene (transposon Tn903) was isolated from plasmid pACYC177 (New England Biolabs, Inc.) using standard PCR techniques. Specifically, a 5′ PCR primer (“pMaxVax primer 3”), ggcttctcacagagtggcgcgccgtgtctcaaaatctct (SEQ ID NO:8), comprising sequences homologous to the 5′ kanamycin gene and an additional DraIII site upstream of an AscI site, and a 3′ primer (“pMaxVax primer 4”), ttgctcagctagcgccggcgccgtcccgtcaagtcagcgt (SEQ ID NO:9), comprising sequences homologous to the 3′ kanamycin gene and a NgoMIV cloning site, were used to amplify the Kana^{r} gene from pACYC177. The PCR reactions, product purification and digest with DraIII and NgoMIV were performed as described above. About 20 ng of each of the Kana^{r} PCR product and ColE1 PCR product were obtained and ligated in a 20 μl reaction, containing 2 μl 10× buffer and 1U ligase (Roche). Amplification in E. coli was performed using standard procedures as described in Sambrook, supra. Plasmids were purified with the QiaPrep-spin Miniprep kit (Qiagen) following the manufacturer's instructions and digested with BsrGl and DraIII for subsequent ligation of the mammalian transcription unit (promoter and polyA). The Kana^{R} gene is used typically for in vivo and/or in vitro studies. Alternative antibiotic resistant, genes, such as ampillicin, tetracycline, and blasticidin resistant genes, cans be used and incorporated into the vector of the invention for in vivo and/or in vitro studies in a variety of cell cultures.

In this example, the human CMV Towne promoter/enhancer was used for driving the expression of the exogenous nucleotide sequence or transgene in mammalian cells. Alternatively, other CMV promoters or non-naturally occurring recombinant or chimeric CMV promoters can be used; for example, a chimeric or recombinant promoter, including an optimized CMV promoter, as described in copending, commonly assigned PCT application Ser. No. 01/20,123 (WO 02/00897), supra, can be used, which is incorporated herein by reference in its entirety for all purposes. Different strains of CMV can be obtained from ATCC. Strains AD169 (VR-538; Rowe, W. (1956) Proc. Soc. Exp. Biol. Med. 145:794-801) and Towne (VR-977; Plotkin, S. A. (1975) Infect. Immun. 12:521-27) were isolated from human patients with CMV infections, while strains 68-1 (Asher, D. M. (1969) Bacteriol. Proc. 269:91) and CSG (Black, H. (1963) Proc. Soc. Exp. Biol. Med. 112:601) were isolated from Rhesus and Vervet monkeys, respectively. The polynucleotide sequences of Towne and AD169 CMV promoters are known in the art (see, e.g., WO 02/00897). Other viral promoters, e.g., from RSV and SV40 virus, and cellular promoters, such as the actin and SRα promoter, and the like, and other promoters known to those of skill in the art, confer ubiquitous transcription in mammalian cells as well. For cell type-specific transcription, the use of cell type-specific promoters, such as muscle specific, liver specific, keratinocyte specific, and the like, and others known to those of skill in the art can be used.

In one embodiment, the pMaxVax10.1 vector comprises a CMV immediate early enhancer promoter (CMV IE), which was isolated from DNA of the CMV virus, Towne strain, by standard PCR methods. The cloning sites EcoRI and BamHI were incorporated into the PCR forward and reverse primers. The EcoRI and BamHI digested CMV IE PCR fragment was cloned into pUC19 for amplification. The CMV promoter was isolated from the amplified pUC19 plasmid by restriction digest with BamHI and BsrGI. The BsrGI site is located 168 bp downstream of the 5′ end of the CMV promoter, resulting in a 1596 bp fragment, which was isolated by standard gel purification techniques for subsequent ligation.

In one embodiment, a polyadenylation signal from the bovine growth hormone (BGH) gene was used. Other poly A signals, which one of skill would understand may be employed, include, e.g., poly A signal sequences from, e.g., SV40 poly A sequences, Herpes simplex Tk, and rabbit beta globin, and the like, and others known to those of skill in the art.

In this instance, a BGH nucleotide sequence was isolated from the pCDNA3.1 vector (Invitrogen) by standard PCR techniques. Briefly, a 5′ PCR forward primer (“pMaxVax primer 5”), agatctgtttaaaccgctgatcagcctcgactgtgccttc (SEQ ID NO:10), which comprises recognition sites for the restriction enzymes PmeI and BglII to form part of the p.MaxVax10.1 vector polylinker, and a 3′ reverse primer (“pMaxVax primer 6”), acctctaaccactctgtgagaagccatagagcccaccgca (SEQ ID NO:11), which comprises a DraIII site for cloning to the minimal plasmid Col/Kana, were prepared by standard techniques and used to amplify a BGH polyA PCR product. The BGH polyA PCR product was diluted 1:100. 1 μl of the diluted BGH polyA PCR product was used as a template for a second PCR amplification using the same 3′ reverse primer and a second 5′ primer (“pMaxVax primer 7”), ggatccggtacctctagagaattcggcggccgcagatctgtttaaaccgctga (SEQ ID NO:12), which overlapped the 5′ end of the template by 20 bp, and contained another 40 bp 5′ sequence comprising BamHI, KpnI, XbaI, EcoRI, and NotI restriction sites for inclusion of these sites in the pMaxVax10.1 vector polylinker. One of skill in the art will understand that a variety of polylinkers can be integrated into the nucleotide sequence of pMaxVax10.1 vector and used to allow for incorporation of one or more additional (exogenous) polynucleotide sequences into the vector at the cloning site(s).

An alternative PCR product was generated with different 5′forward PCR primers to generate a vector with a modified polylinker to facilitate usage of BamHI and KpnI cloning sites (see, e.g., FIG. 3). The orientation of the restriction sites in this polylinker is 5′-3′: BamHI, XhaI, KpnI, EcoRI, NotI, BglII, and PmeI. The polylinker sequence is: ggatccactcatctagaacaatggtaccaatacgaattcggcggccgcagatctgtttaaacc. The PCR products were digested with BamHI and DraIII and gel purified.

The final ligation reaction to form pMaxVax10.1 was performed with about 20 ng each of the BsrG1 and BamHI digested CMV IE PCR product, BamHI and DraIII digested polylinker and BGH poly A PCR product, and the DraIII and BsrG1 digested minimal plasmid Col/Kana in a 50 μl reaction with 5 μl 10× ligase buffer and 2U ligase (Roche). Ligation, amplification and plasmid purification were performed as described above. The plasmid was transfected into E. coli using standard techniques for cloning.

### Example 2

Construction of Vector pMaxVax with an Exogenous Polynucleotide Sequence

An exogenous nucleotide sequence encoding an exogenous polypeptide of interest can be isolated by PCR with BamHI and KpnI restriction enzyme recognition sequences in the PCR forward and reverse primer as described above. In this example, a polynucleotide sequence encoding a CD28 receptor binding protein (“CD28BP”) polypeptide (e.g., CD28BP-15 polypeptide, which is polypeptide sequence SEQ ID NO:66 and is encoded by, e.g., nucleic acid sequence SEQ ID NO:19 as shown in PCT application Ser. No. 01/19,973, which published with International Publication No. WO 02/00717), is incorporated into the expression pMaxVax10.1 vector. PCT application Ser. No. 01/19,973 (WO 02/00717) is incorporated herein by reference in its entirety for all purposes. To verify the correct sequence of the PCR products, the fragments are cloned conveniently into the TOPO® cloning vectors (Invitrogen) for sequencing according to the manufacturer's protocols. After BamHI and KpnI digestion and gel purification, the genes are cloned into a mammalian expression vector to confirm the expression of the gene. To clone the genes into the polylinker of pMaxVax, the vector pMaxVax 10.1 with modified polylinker as described above was digested with BamHI and KpnI, gel purified and ligated to the respective genes, as described above. The expression construct (see FIG. 3), which comprises the nucleotide sequence encoding a CD28BP polypeptide. (in this example, nucleic acid sequence SEQ ID NO:19 as described in WO 02/00717), can be used for in vivo and in vitro expression in human and other mammalian cells and other cells in culture, including non-mammalian cells and the like.

The nucleotide sequence of an exemplary pMaxVax10.1 expression vector, which comprises an exogenous polynucleotide sequence that encodes CD28BP, is set forth in SEQ ID NO:3.

One of skill will also understand the above procedure can be readily adapted to construct an expression vector comprising different vector components, such as different promoters, signal sequences, termination sequences, replication origin sequences, resistant gene or marker sequences, and/or one or more additional exogenous nucleotide sequences of interest.

### Example 3

Bicistronic Expression Vector

The invention also includes a pMaxVax10.1 bicistronic expression vector that comprises at least two cloning sites with selected polylinkers for incorporating at least two exogenous nucleotides encoding at least two exogenous polypeptides of interest. See, e.g., FIG. 4.

Although a wide variety of exogenous nucleotides can be incorporated into the expression vector of the invention, in this example the incorporation of a first exogenous nucleotide sequence encoding a co-stimulatory polypeptide (e.g., CD28BP-15 as described above) and a second exogenous sequence encoding an antigen (e.g., a cancer antigen) into an expression vector of the invention is described.

For immunotherapy studies it is desirable to express the immunostimulatory molecule in the same cells as, for example, a cancer antigen. A nucleotide sequence encoding a cancer antigen, such as EpCAM/KSA or a mutant or variant thereof, can be cloned into an expression vector (FIGS. 1 or 2) to generate a pMaxVax10.1-EpCAM/KSA vector, using a procedure analogous to that described above for cloning the CD28BP polynucleotide sequence into the pMaxVax vector backbone. Two expression constructs, e.g., the pMaxVax10.1-CD28BP vector (or other pMaxVax vector) and the pMaxVax10.1-EpCAM/KSA vector (or other pMaxVax vector including a nucleotide sequence encoding an antigen), can then be co-transfected in cell culture or co-administered in vivo to a subject in need of such therapeutic or prophylactic treatment.

In an alternative format, which may be an optimal format for some therapeutic or prophylactic applications, both the EpCAM/KSA (or a EpCAM/KSA mutant or variant thereof) and CD28BP-encoding or other co-stimulatory polypeptide-encoding polynucleotides (or a different antigen gene and/or co-stimulatory polypeptide-encoding polynucleotide) can be expressed from the same vector. In one format, the resulting antigen and CD29BP polypeptides can be co-expressed from a single promoter linked by an internal ribosomal entry site (e.g., IRES bicistronic expression vectors, Clontec). This example describes the construction of an exemplary bicistronic vector for expression of at least one CD28BP-encoding polypeptide and at least one antigen or antigen fragment, such as an EpCAM/KSA antigen (or alternatively a polynucleotide encoding a second co-stimulatory polypeptide), in which the CD28BP-encoding polynucleotide (or polynucleotide encoding another co-stimulatory polypeptide) and the nucleotide sequence encoding the antigen or antigen fragment (or alternatively a polynucleotide encoding a second co-stimulatory polypeptide) form two separate expression units. In particular, this example describes the construction of a bicistronic vector for expression of CD28BP (e.g., CD28BP-15) and the cancer antigen EpCAM/KSA (or mutant or variant thereof) in which the CD28BP-15 encoding polynucleotide and the polynucleotide encoding the cancer antigen or antigen fragment form two separate expression units, each regulated by its own respective promoter and poly A signal. One of skill will understand that this procedure can also be readily adapted to construct a bicistronic vector comprising at least one CD28BP-encoding polynucleotide (or fragment or fusion protein thereof as described in WO 02/00717) and a different antigen or antigen fragment (or a different co-stimulatory polypeptide). The CD28BP-15-encoding polynucleotide is inserted into the polylinker of a pMaxVax10.1 vector as described above, forming the first expression unit. The nucleic acid sequence of the cancer antigen, here the polynucleotide encoding the extracellular domain of EpCAM/KSA (or mutant or variant thereof), is linked to a second mammalian expression promoter (exemplary promoters include those set forth in this Example above and elsewhere) and a second poly A signal (exemplary signals include those set forth in this Example above and elsewhere) to form the second expression unit.

The second expression unit can be cloned into 3 different sites in the construct pMaxVax-CD28BP, both in forward or reverse orientation: (i) downstream of the first expression unit (e.g., CMV promoter-CD28BP-SPA polyA, CMVpromoter-CD28BP-BGH polyA. or CMVpromoter-CD28BP-SV40 polyA) using the single cloning sites DraIII and AscI in pMaxVax10.1; (ii) between the ColE1 and Kana^{r} gene using the single restriction sites NgoMI and NheI; (iii) between the Kana^{r} gene and the CMV promoter into the single EcoRV and BsrGI restriction sites (see vector description above in this Example). Independent of the location of the second expression unit, it is advisable to add a terminator sequence downstream of the first expression unit. A consensus terminator sequence 5′-ATCAAAA/TTAGGAAGA3′ is described in Ming-Chei Maa et al. (1990) JBC 256 (21):12513-12519. In the construct pMaxVax10.1-CD28BP the sequence can be placed into the single DraIII site downstream of the poly A sequence (e.g., synthetic poly A (SPA) nucleotide sequence, BGH poly A sequence, or SV40 poly A sequence).

This example describes the cloning strategy of the second expression unit for location (ii). The second promoter (e.g., a WT CMV promoter, such as human CMV promoter or a recombinant CMV promoter or shuffled CMV promoter (as, e.g., described in PCT application Ser. No. 01/20,123, which published with International Publication No. WO 02/00897, which is incorporated herein by reference in its entirety for all purposes) with improved expression activity), the EpCAM/KSA cancer antigen (or mutant or variant thereof), and the second poly A are isolated from the respective template plasmids by PCR or assembled from oligonucleotides (as described above in this Example). The PCR primers are designed to contain single restriction sites, which allow for partial site-directed cloning of the three fragments into the final vector. The 5′forward PCR primer for isolation of the shuffled CMV promoter contains the single NgoMIV (also called NgoMI) cloning site. The 3′reverse primer contains the NgoMIV site and another restriction enzyme site, which does not cut in any of the other vector units (i.e., AccI, AgeI, AvrII, BsU361, MluI, RsrII, SalI) upstream of it separated by a spacer of at least 10 base pairs. In the example AccI is chosen as the additional cloning site. The PCR product is digested with NgoMIV followed by gel purification and cloned into the NgoMIV linearized and gel purified pMaxVax10.1-CD28BP. The correct orientation of the second CMV promoter after ligation is determined by PCR from bacterial colonies (as described in Sambrook, supra) using the 3′reverse primer and any forward primer of choice located about 500-600 bp upstream of the reverse primer in the CMV promoter sequence. The second promoter containing plasmid is then digested with AccI and NheI for cloning of the cancer antigen. The 5′primer for the EpCAM/KSA cancer antigen (or mutant or variant thereof) contains the single AccI site and the 3′primer the single NheI site and an additional single restriction site upstream, AgeI, separated by a spacer of at least 10 base pairs. The PCR product is digested with the enzymes AccI and NheI and cloned into the equally digested vector. The resulting construct is digested AgeI and NheI for cloning of the SV40 polyA/terminator sequence fragment or BGH polyA terminator sequence. The 5′ forward primer for this PCR product contains the single AgeI site and the 3′reverse primer the terminator sequence followed by the single NheI site. The 5′ cloning sequence and the NheI site are incorporated in the oligonucleotides. The resulting (e.g., double-stranded) AgeI/NheI poly A fragment is then cloned in the equally digested vector. The cloning strategy is outlined below.

1) NgoMIV<CMV promoter>AccI/NgoMIV

2) AccI<EpCAM/KSA>AgeI/NheI

3) AgeI<BGH polyA>NheI

One of skill will also understand the above procedure can be readily adapted to construct an expression vector comprising different vector components, such as different promoters, signal sequences, termination sequences, replication origin sequences, resistant gene or marker sequences, and/or one or more additional exogenous nucleotide sequences of interest.

### Example 4

DNA Plasmid Amplification in E. Coli

The DNA plasmids described in Examples 1-3 above and other nucleic acids of the invention may be amplified in E. coli as follows. The DNA plasmids are transformed into XL1-blue-mrf′ (Stratagene) electro-competent bacteria and plated over night on agar plates, containing Kanamycin at a final concentration of 40 μg/ml. Single colonies are grown as a starter culture in 2 ml LB media (10 g of Tryptone, 5 g of Yeast Extract, 10 g of NaCl per liter of DDH₂O), supplemented with Kanamycin at a final concentration of 40 μg/ml, for 5 hours in a shaker at 37° C. The starter cultures are diluted 1:1000 into new 200-500 ml cultures of such selective LB media and further grown for 14-16 hrs. The bacterial cultures are pelleted by centrifugation, and the plasmids are purified (Qiagen Endofree Plasmid purification kit) and dissolved in endotoxin free PBS (Sigma) at a final concentration of 1 μg/μl.

One of skill will understand that a similar procedure can be used to construct an expression vector comprising a nucleotide sequence encoding a CTLA-4 receptor binding protein (“CTLA-4BP”) in place of the sequence encoding CD28BP above. Nucleotide sequences encoding a variety of novel CTLA-4BP polypeptides are set forth in as described in commonly assigned PCT application Ser. No. 01/19,973, published with International Publication No. WO 02/00717, filed Jun. 22, 2001. Such a vector can comprise a bicistronic vector, if desired, with a second nucleotide sequence of interest (e.g., encoding an antigen or another co-stimulatory molecule) included in the position occupied above by the antigen (see also FIGS. 22A-22B in WO 02/00717).

### Example 5

Use of pMaxVax10.1 Vector for Protein Expression

The pMaxVax10.1 (“pMV10.1”) vector can be used for expression of a heterologous protein by incorporating the nucleotide sequence encoding such protein into the pMV10.1 vector at the cloning site (see, e.g., FIG. 1) as discussed above using well known cloning techniques. In this example, an antigenic polypeptide of a wild-type dengue virus is cloned into the pMV10.1 expression vector and the vector is used to express the antigen. Upon administration of the vector to a subject (e.g., mammal), an immune response is induced against the expressed antigen in the serum of the subject. This example demonstrates that the vectors of the invention are useful for expression of one or more heterologous protein(s), where the nucleotide sequence encoding each such protein is cloned into the expression vector. This example also illustrates that vectors of the invention are useful as DNA vaccines or in DNA vaccine or protein vaccine formats via the incorporation at least one polynucleotide encoding at least one antigen of interest in the vector. Optionally, at least one nucleotide sequence encoding an immunomodulatory polypeptide, adjuvant, and/or additional antigen can also be cloned into the expression vector to enhance or augment the in vivo cellular and/or humoral immune response.

Dengue (DEN) viruses are known among flaviviruses as agents of disease in humans. Dengue viruses comprise four known distinct, but antigenically related serotypes, named Dengue-1 (DEN-1 or Den-1), Dengue-2 (DEN-2 or Den-2), Dengue-3 (DEN-3 or Den-3), and Dengue-4 (DEN-4 or Den-4). Dengue virus particles are typically spherical and include a dense core surrounded by a lipid bilayer. FIELDS VIROLOGY, supra.

The genome of a dengue virus, like other flaviviruses, typically comprises a single-stranded positive RNA polynucleotide. FIELDS VIROLOGY, supra, at 997. The genomic RNA serves as the messenger RNA for translation of one long open reading frame (ORF) as a large polyprotein, which is processed co-translationally and post-translationally by cellular proteases and a virally encoded protease into a number of protein products. Id. Such products include structural proteins and non-structural proteins. A portion of the N-terminal of the genome encodes the structural proteins—the C protein, prM (pre-membrane) protein, and E protein—in the following order: C-prM-E. Id. at 998. The C-terminus of the C protein includes a hydrophobic domain that functions as a signal sequence for translocation of the prM protein into the lumen of the endoplasmic reticulum. Id. at 998-999. The prM protein is subsequently cleaved to form the structural M protein, a small structural protein derived from the C-terminal portion of prM, and the predominantly hydrophilic N-terminal “pr” segment, which is secreted into the extracellular medium. Id. at 999. The E protein is a membrane protein, the C-terminal portion of which includes transmembrane domains that anchor the E protein to the cell membrane and act as signal sequence for translocation of non-structural proteins. Id. The E protein is the major surface protein of the virus particle and is believed to be the most immunogenic component of the viral particle. The E protein likely interacts with viral receptors, and antibodies that neutralize infectivity of the virus usually recognize the E protein. Id. at 996. The M and E proteins have C-terminal membrane spanning segments that serve to anchor these proteins to the membrane. Id. at 998.

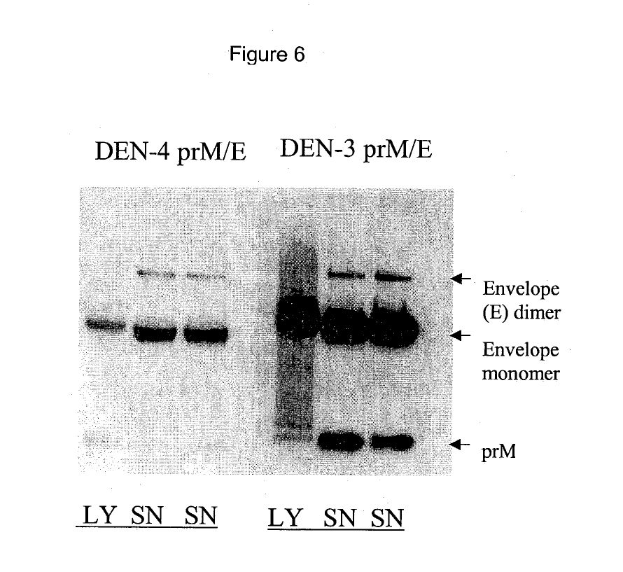

The polynucleotide sequence coding for each of the viral DEN-3 and DEN-4 membrane (prM) and envelope (E) antigens (DEN-3 prM/E and DEN-4 prM/E) was inserted into the pMV10.1 expression vector. Each resulting vector comprising the heterologous antigen-encoding polynucleotide sequence (e.g., pMV10.1_{DEN-3 prM/E} vector and pMV10.1_{DEN-4 prM/E} vector) was transfected into a population of human HEK 293 cells. See FIG. 6. As shown in the figure, a dengue virus antigen was each expressed from each respective vector in mammalian cells in vitro. The prM/E antigenic proteins expressed in the cell lysates (Ly) and the medium supernatants (SN) were separated by gel electrophoresis, blotted to nitrocellulose filters, and analyzed by Western Blot with DEN-3 and DEN-4 serotype specific antibodies. The results illustrate expression of each of the dengue virus antigens using the pMV10.1 vector and demonstrate that the vector is useful as an expression vector for expression of a heterologous protein following insertion of the nucleotide sequence encoding the heterologous protein into the pMV10.1 vector.

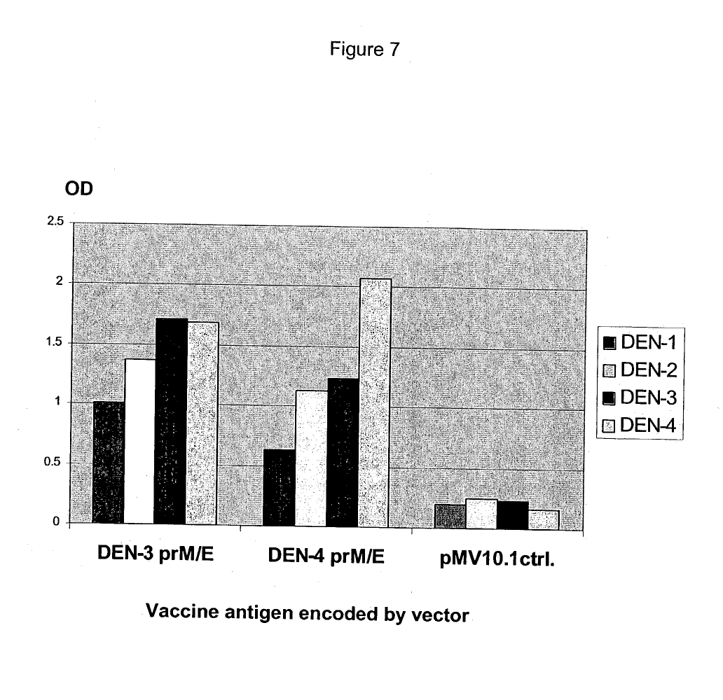

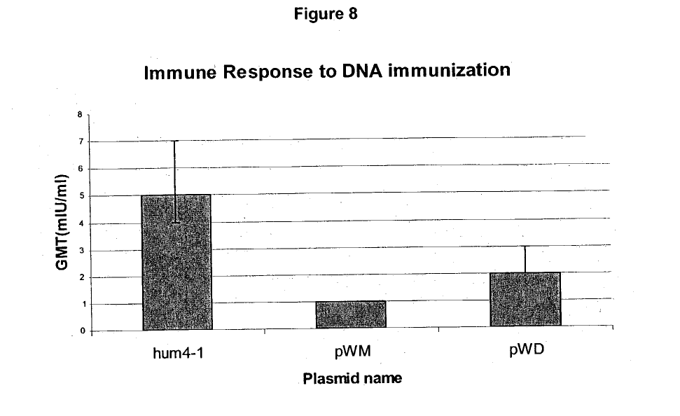

To test for the ability of the antigens expressed by these pMV 10.1 vectors to induce an in vivo immune response in a mammal, separate groups of mice were immunized by intramuscular injection with 100 micrograms (ug) of each of the following plasmid vectors at days 0, 14, 28, and 56:1) pMV10.1 expression vector encoding the DEN-3 prM/E antigen; 2) pMV10.1 expression vector encoding the DEN-4 prM/E antigen; or 3) pMV10.1 expression vector alone with no heterologous antigen-encoding polynucleotide sequence (pMV10.1 control), which served as the control vector. Serum was collected from the mice at day 90 and analyzed for DEN-specific antibody induction in ELISA plates coated with DEN-1, DEN-2, DEN-3 and DEN-4 serotype specific antigens. FIG. 7 illustrates optical density (OD) values (y-axis) obtained following DEN-specific antibody induction in mouse serum using ELISA plates coated with DEN-1, DEN-2, DEN-3 and DEN-4 serotype specific antigens. On the x-axis is shown the particular antigen expressed by the administered pMV10.1 vector (or no antigen as for the pMV10.1 control). These results confirm the expression of two wild-type dengue virus antigens, DEN-3 prM/E and DEN-4 prM/E, from the pMV10.1 vector in vivo, as determined by antibody induction in mice and serum analyses by ELISA assays.

### Example 6

Use of pCMV-Mkan Vector for Expression of Hepatitis Virus Surface Antigen and DNA Vaccination

This example illustrates the use of the pCMV-Mkan expression vector for in vitro and in vivo expression of various hepatitis surface antigens. This example also demonstrates the use of the vector as a DNA vaccine to induce an in vivo immune response in a mammal through in vivo expression and production of the antigen.

The following 3 vectors were constructed using the pCMV-Mkan expression vector for the plasmid backbone: (1) plasmid hum1-4; (2) plasmid pWM; (3) plasmid pWD. The plasmid hum4-1 is a pCMV-Mkan expression vector comprising a heterologous polynucleotide sequence that encodes the wild-type human Hepatitis B Virus Envelope (antigen). The plasmid pWM is a pCMV-Mkan expression vector comprising a heterologous polynucleotide sequence that encodes the Woolly Monkey (WM) Hepatitis Virus Envelope (antigen). The plasmid pWD is a pCMV-Mkan expression vector comprising a heterologous polynucleotide sequence that encodes the Woodchuck Hepatitis Virus Envelope (antigen). The polypeptide and polynucleotides sequences of human Hepatitis B surface antigen envelope, Woodchuck Hepatitis Virus Envelope antigen, and Woolly Monkey Hepatitis Virus Envelope antigen are well known in the art. For the Hepatitis virus sequences for Woolly monkey and Woodchuck Hepatitis Virus, see Genbank Accession Nos. AF046996 and J04514 (strain WHV8), respectively. See also commonly assigned U.S. Pat. No. 6,541,011, including FIGS. 17-18 therein.

For each vector, the heterologous antigen-encoding polynucleotide sequence was cloned into the pCMV-Mkan polynucleotide sequence (SEQ ID NO:4) at the stuffer nucleotide sequence segment cloning site (see FIG. 5) using standard cloning techniques well known in the art.

Expression of an envelope antigen from the vector encoding the antigen in mammalian cells in vitro was analyzed and confirmed by using standard Western Blot techniques (data not shown). For example, the plasmid hum4-1 (pCMV-Mkan vector further comprising the polynucleotide sequence encoding the wild-type human Hepatitis B surface antigen envelope) was transfected into Cos-7 cells (ATCC #CRL-1651) using SuperFect transfection reagent as described by the manufacturer (Qiagen). Supernatant from these cells was collected at 24 hrs and analyzed by Western Blot using stained a goat anti-HBs antibody (Dako #B0560) for detection. Significant Hepatitis B envelope protein was produced by pCMV-Mkan expression plasmid comprising the heterologous polynucleotide sequence encoding the human Hepatitis B surface antigen envelope.

Each plasmid vector was tested for its ability to induce an in vivo immune response in a mammal through in vivo expression and production of an amount of the heterologous Hepatitis antigen sufficient to induce a detectable immune response.

For each mammalian test group, 30 six-week old C57BL/6 mice were anesthetized and injected i.m. with 50 microliters DNA solution comprising one of the expression vectors described above (and shown in FIG. 8) in phosphate-buffered saline (PBS). I.m. injection was performed in the tibialis anterialis muscle of each leg muscle of each mouse (10 micrograms DNA total administered per mouse). Serum obtained from each mouse was analyzed 4 weeks after administration for the level of anti-Hepatitis B antibody(ies) as measured by Abbott AUZYME ELISA assays (expressed as the geometrical mean titer ±SEM for each group in International Units (IU) per milliliter (ml)). The results, which are shown in FIG. 8, demonstrate that the expression vector is capable of expressing a variety of heterologous polynucleotide sequences, each of which encodes a polypeptide of interest. Furthermore, the results demonstrate that a pCMV-Mkan vector that further comprise an antigenic polypeptide (such as a Hepatitis antigen) can be used effectively in mammals to induce an in vivo immune response against the antigen and thus can function successfully as a DNA vaccine.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. For example, all the techniques and apparatus described above may be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

What is claimed is:

1. An isolated or recombinant nucleic acid comprising a polynucleotide sequence that has at least about 90% nucleic acid sequence identity to a polynucleotide sequence selected from the group of SEQ ID NOS:1, 2 and 5, or a complementary polynucleotide sequence thereof.

2. The isolated or recombinant nucleic acid of claim 1, wherein the polynucleotide sequence has at least about 95% nucleic acid sequence identity to a polynucleotide sequence selected from the group of SEQ ID NOS:1, 2, and 5, or a complementary polynucleotide sequence thereof.

3. The isolated or recombinant nucleic acid of claim 1, wherein the polynucleotide sequence comprises a polynucleotide sequence selected from the group of SEQ ID NOS:1, 2, and 5, or a complementary polynucleotide sequence thereof.

4. The isolated or recombinant nucleic acid of claim 1, comprising a polynucleotide sequence which hybridizes under at least stringent conditions over substantially the entire length of the polynucleotide sequence of SEQ ID NO:1, 2 or 5, or a complementary polynucleotide sequence thereof.

5. The nucleic acid of claim 1, wherein the nucleic acid is DNA.

6. The nucleic acid of claim 5, wherein the nucleic acid comprises a promoter and terminator signal sequence.

7. The nucleic acid of claim 6, wherein the nucleic acid further comprises an origin of replication.

8. The nucleic acid of claim 7, wherein the origin of replication is a ColE1 origin of replication.

9. The nucleic acid of claim 6, wherein the nucleic acid comprises a polynucleotide sequence encoding a kanamycin resistance marker.

10. The nucleic acid of claim 6, wherein the terminator signal sequence is a BGH polyadenylation sequence.

11. The nucleic acid of claim 6, wherein the promoter is a CMV promoter or a variant thereof.

12. The nucleic acid of claim 12, wherein the promoter is a chimeric CMV promoter.

13. The nucleic acid of claim 12, wherein the promoter is a shuffled CMV promoter.

14. The nucleic acid of claim 1, further comprising at least one polylinker.

15. The nucleic acid of claim 1, further comprising at least one restriction site for insertion of a polynucleotide sequence encoding a polypeptide.

16. The nucleic acid of claim 1, wherein the nucleic acid is an expression vector capable of expressing at least one exogenous polypeptide upon incorporation into the expression vector of a polynucleotide encoding the at least one exogenous polypeptide.

17. The nucleic acid of claim 16, wherein the at least one exogenous polynucleotide sequence is operably linked to a promoter polynucleotide sequence present in the nucleic acid.

18. The nucleic acid of claim 1, wherein the nucleic acid further comprises at least one polynucleotide sequence encoding at least one antigen, co-stimulatory polypeptide, adjuvant, chemokine, or cytokine, or any combination thereof.

19. The nucleic acid of claim 18, wherein the at least one antigen comprises at least one viral antigen.

20. The nucleic acid of claim 19, wherein the at least one viral antigen comprises at least one flavivirus virus antigen.

21. The nucleic acid of claim 18, wherein the at least one antigen induces an immune response against at least one serotype of a dengue virus selected from dengue-1, dengue-2, dengue-3, and dengue-4.

22. The nucleic acid of claim 18, wherein the at least one antigen comprises at least one cancer antigen.

23. The nucleic acid of claim 22, wherein the at least one cancer antigen comprises EpCAM/KSA or a mutant or variant thereof.

24. The nucleic acid of claim 22, wherein the cancer antigen induces an immune response against human epithelial cell adhesion molecule (EpCAM).

25. The nucleic acid of claim 22, wherein the cancer antigen induces production of antibodies against human EpCAM.

26. The nucleic acid of claim 23, wherein the nucleic acid further comprises at least one polynucleotide sequence encoding at least one co-stimulatory polypeptide.

27. The nucleic acid of claim 26, wherein the nucleic acid comprises a promoter polynucleotide sequence and the at least one polynucleotide sequence encoding at least one co-stimulatory polypeptide is operably linked to the promoter sequence.

28. The nucleic acid of claim 26, wherein the at least one co-stimulatory polypeptide binds a mammalian CD28 receptor.

29. The nucleic acid of claim 28, wherein the at least one co-stimulatory polypeptide comprises a B7-1 variant.

30. The nucleic acid of claim 27, wherein the nucleic acid comprises the expression vector shown in FIG. 4.

31. The nucleic acid of claim 18, said nucleic acid further comprising at least one exogenous polynucleotide sequence encoding at least one co-stimulatory polypeptide.

32. The nucleic acid of claim 31, wherein the at least one polynucleotide sequence encoding the at least one co-stimulatory polypeptide is operably linked to a promoter sequence present in the nucleic acid.

33. The nucleic acid of claim 32, wherein the at least one co-stimulatory polypeptide binds human CD28 receptor.

34. The nucleic acid of claim 32, wherein the at least one co-stimulatory polypeptide binds human CTLA-4 receptor.

35. The nucleic acid of claim 32, wherein the at least one co-stimulatory polypeptide comprises a B7-1 variant.

36. The nucleic acid of claim 1, wherein said nucleic acid is a synthetic nucleic acid.

37. An isolated or recombinant nucleic acid comprising a polynucleotide sequence that has at least about 90% nucleic acid sequence identity to the polynucleotide sequence of SEQ ID NO:3 or 4, or a complementary polynucleotide sequence thereof.

38. The nucleic acid of claim 37, wherein the polynucleotide sequence has at least about 95% nucleic acid sequence identity to the polynucleotide sequence of SEQ ID NO:3 or 4, or a complementary polynucleotide sequence thereof.

39. The nucleic acid of claim 38, wherein the polynucleotide sequence comprises a polynucleotide sequence selected from the group of SEQ ID NOS:3 and 4, or a complementary polynucleotide sequence thereof.

40. The isolated or recombinant nucleic acid of claim 37, comprising a polynucleotide sequence that hybridizes under at least stringent conditions over substantially the entire length of the polynucleotide sequence of SEQ ID NO:3 or 4, or a complementary polynucleotide sequence thereof.

41. The nucleic acid of claim 37, wherein the nucleic acid is DNA.

42. The nucleic acid of claim 1, wherein the nucleic acid is an expression vector capable of expressing at least one exogenous polypeptide upon incorporation into the expression vector of a polynucleotide encoding the at least one exogenous polypeptide.

43. The nucleic acid of claim 16, wherein the at least one exogenous polynucleotide sequence is operably linked to a promoter polynucleotide sequence present in the nucleic acid.

44. A nucleic acid vector comprising the nucleic acid of claim 1.

45. The nucleic acid vector of claim 44, said nucleic acid vector comprising a promoter, wherein said vector further comprises a heterologous nucleic acid coding sequence that encodes at least one polypeptide, said heterologous nucleic acid coding sequence operably linked to the promoter.

46. A nucleic acid vector comprising the nucleic acid of claim 37.

47. A nucleic acid vector comprising a polynucleotide sequence that hybridizes under at least stringent conditions over substantially the entire length of a polynucleotide sequence selected from the group of SEQ ID NOS:1-5, or a complementary polynucleotide sequence thereof.

48. An isolated expression vector construct for the expression of a polypeptide in a mammalian cell, the expression vector comprising:
(a) a first polynucleotide sequence having at least 90% nucleic acid sequence identity to a polynucleotide sequence selected from the group of SEQ ID NOS:1, 2, and 5, wherein said first polynucleotide comprises a promoter for expression of the polypeptide in a mammalian cell and a terminator signal sequence; and
(b) a second polynucleotide sequence encoding the polypeptide, wherein said second nucleic acid sequence is operably linked to the promoter.

49. A vector comprising the vector plasmid map shown in FIGS. 1, 2, 3, 4, or 5.

50. A-DNA vaccine vector comprising the nucleic acid vector of claim 44, wherein said nucleic acid vector further comprises at least one polynucleotide sequence encoding at least one antigen and optionally further comprises at least one polynucleotide sequence encoding at least one co-stimulatory polypeptide.

51. A DNA vaccine vector comprising the nucleic acid of claim 37.

52. A composition comprising at least one nucleic acid of claim 1 and a carrier.

53. The composition of claim 52, wherein the composition is a pharmaceutical composition and the carrier is a pharmaceutically acceptable carrier.

54. A composition comprising at least one nucleic acid of claim 37 and a carrier.

55. The composition of claim 54, wherein the composition is a pharmaceutical composition and the carrier is a pharmaceutically acceptable carrier.

56. A composition comprising at least one nucleic acid vector of claim 44 and an excipient.

57. A composition comprising at least one nucleic acid vector of claim 46 and an excipient.

58. The composition of claim 56, wherein the composition is a pharmaceutical composition and the excipient comprises a pharmaceutically acceptable excipient.

59. The composition of claim 57, wherein the composition is a pharmaceutical composition and the excipient comprises a pharmaceutically acceptable excipient.

60. A host cell comprising at least one nucleic acid of claim 1.

61. The cell of claim 60, wherein the cell is a eukaryotic cell.

62. A host cell comprising at least one nucleic acid of claim 37.

63. A composition comprising the host cell of claim 60 and an excipient.

64. A mammalian cell transformed with at least one nucleic acid vector of claim 44.

65. A mammalian cell transformed with at least one nucleic acid vector of claim 46.

66. A method for expressing a polypeptide, comprising:
(a) providing a cell comprising at least one vector of claim 44, said at least one vector further comprising a polynucleotide coding sequence that encodes the polypeptide; and
(b) culturing said cell under conditions suitable for expression of the polypeptide.

67. A process for transfecting a cell, said process comprising contacting said cell with a vector of claim 44 under conditions for transfection of the cell with said vector.

68. A method of expressing a polypeptide, the method comprising:
(a) introducing into a population of cells a nucleic acid of claim 1, which nucleic acid further comprises a polynucleotide sequence that encodes the polypeptide, said polynucleotide sequence operatively linked to a regulatory sequence effective to produce the encoded polypeptide;
(b) culturing the cells in a culture medium to express the polypeptide.

69. The method of claim 68, further comprising isolating the polypeptide from the cells or from the culture medium.

70. A method of producing a polypeptide, the method comprising:
(a) introducing into a population of cells an expression vector comprising the nucleic acid of claim 1, said nucleic acid further comprising a polynucleotide sequence that encodes the polypeptide, said polynucleotide sequence operatively linked to a promoter sequence within the nucleic acid to produce the encoded polypeptide;
(b) administering the expression vector into a mammal; and
(c) isolating the polypeptide from the mammal or from a byproduct of the mammal.

72. A monocistronic expression vector comprising the nucleic acid of claim 1.

73. The monocistronic expression vector of claim 72, wherein the vector further comprises a polynucleotide sequence encoding a CD28 binding protein.

73. The monocistronic expression vector of claim 72, wherein the vector comprises that shown in FIG. 3.

74. A bicistronic expression vector as shown in FIG. 4.

75. A method for inducing an immune response in a subject, comprising:
administering to the subject at least one nucleic acid of claim 1, wherein said nucleic acid comprises a mammalian promoter sequence and further comprises a polynucleotide sequence encoding an antigenic polypeptide that is operatively linked to the mammalian promoter sequence, said nucleic acid being administered in an amount sufficient to induce an immune response by expression of the polypeptide.

76. A method for enhancing an immune response to an antigen in a subject, comprising administering to the subject a vector of claim 37, wherein said vector further comprises at least one polynucleotide sequence encoding an immunomodulatory or co-stimulatory polypeptide, such that the immune response induced in the subject by the antigen is enhanced by the expressed immunomodulatory or co-stimulatory polypeptide, wherein the an immunomodulatory or co-stimulatory polypeptide is expressed and enhanced the immune response in the subject induced by an antigen.

77. The method of claim 76, wherein an expression vector encoding the antigen is administered to the subject.

78. A method of treating a disorder or disease in a mammal in need of such treatment, comprising administering to the subject a nucleic acid vector of claim 37, said nucleic acid further comprising a polynucleotide sequence that encodes a polypeptide useful in treating said disorder or disease, wherein the polypeptide-encoding polynucleotide sequence e is operatively linked to a mammalian promoter sequence effective to produce the encoded polypeptide, wherein the mammalian promoter sequence comprises a portion of the polynucleotide sequence of the nucleic acid vector, and wherein nucleic acid vector is administered in an amount sufficient to produce an effective amount of the polypeptide to treat said disorder or disease.


SEQUENCE LISTING (13 sequences)

SEQ ID NO: 1 - DNA, 3710 bp, Artificial Sequence
  pMV10.1 DNA expression vector
ggatccggtacctctagagaattcggcggccgcagatctgtttaaaccgctgatcagcctcgactgtgccttctagttaccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctcacagagtggcgcgccgtgtctcaaaatcactgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgccggcgctagctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgatatctgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactctccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgcctcacggggatttccaagtctccaccccattgacgtcaatgggagtttgttttggcaccaaaatcaacgggactttccaaaatgccgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcttataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccgccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcaggggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcctt

SEQ ID NO: 2 - DNA, 3879 bp, Artificial Sequence
  pMV10.1-shuffled CMV DNA expression vector
ggatccggtacctctagagaattcggcggccgcagatctgtttaaaccgctgatcagcctcgactgtgccttctagttaccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctcacagagtggcgcgccgtgtctcaaaatcactgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgccggcgctagctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgatatcatatgaggctatatcgccgatagaggcgacatcaagccggcacatggccaatgcatatcgatctatacattgaatcaatattggcaattagccatattattcattggttatatagcataaatcaatattggctattggccattgcatacgttgtatccgtatcataatatgtacatttatattggcccatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggcgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggtggggcttccacatccgagccctggtcccatgcctccagcgactcatggtcgctcggcagctccttgctcccaacagtggaggccagacttaggcacagcacgatgcccaccaccaccagtgtgccgcacaaggccgtggcggtagggtatgtgtctgaaaatgagctcggagatcgggctcgcaccgctgacgcagatggaagacttaaggcagcggcagaagaagacgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacggactgttcctttccatgggtcttttctgcagtcaccgtcctt

SEQ ID NO: 3 - DNA, 4790 bp, Artificial Sequence
  PMV10.1-CD28BP DNA expression vector
ggatccatgggtcacacaatgaagtggggatcactaccacccaagcgcccatgcctctggctctctcagctcttggtgctcactggtcttttttacttctgttcaggcatcaccccaaagagtgtgaccaaaagagtgaaagaaacagtaatgctatcctgtgattacaacacatccactgaagaactgacaagccttcggatctattggcaaaaggatagtaaaatggtgctggccatcctgcctggaaaagtgcaggtgtggcctgagtacaagaaccgcaccatcactgacatgaacgataacccccgtattgtgatcctggctctgcgcccgtcggacagtggcacctacacctgtgttattcagaagcctgttttgaaaggggcttataaactggagcacctggcttccgtgaggttaatgatcagagctgacttccctgtccctaccataaatgatcttggaaatccatctcctaatatcagaaggctaatttgctcaacctctggaggttttccaaggccccacctctactggttggaaaatggagaagaattaaatgctaccaacacaacagtttcccaagatcctggaactgagctctacatgattagcagtgaactggatttcaatgtgacaaataaccacagcatcgtgtgtctcatcaaatacggggagctgtcggtgtcacagatcttcccttggagcaaacccaagcaggagcctcccattgatcagcttccattctgggtcattatcccagtaagtggtgctttggtgctcactgcggtagttctctactgcctggcctgcagacatgttgcgaggtggaaaagaacaagaaggaatgaagagacagtgggaactgaaaggctgtcccctatctacttaggctctgcgcaatcctcgggctgaggtaccaatacgaattcggcggccgcagatctgtttaaaccgctgatcagcctcgactgtgccttctagttaccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctcacagagtggcgcgccgtgtctcaaaatcactgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgccggcgctagctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgatatcatatgaggctatatcgccgatagaggcgacatcaagccggcacatggccaatgcatatcgatctatacattgaatcaatattggcaattagccatattattcattggttatatagcataaatcaatattggctattggccattgcatacgttgtatccgtatcataatatgtacatttatattggcccatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggcgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggtggggcttccacatccgagccctggtcccatgcctccagcgactcatggtcgctcggcagctccttgctcccaacagtggaggccagacttaggcacagcacgatgcccaccaccaccagtgtgccgcacaaggccgtggcggtagggtatgtgtctgaaaatgagctcggagatcgggctcgcaccgctgacgcagatggaagacttaaggcagcggcagaagaagacgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacggactgttcctttccatgggtcttttctgcagtcaccgtcctt

SEQ ID NO: 4 - DNA, 3741 bp, Artificial Sequence
  pCMV-Mkan DNA plasmid expression vector with Stuffer nucleotide sequence (cloning site)
acatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgatgcagtggaattcggtacctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacagctgcgcatccatcacactggcggccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatggtgtctcaaaatctctgatgttacattgcacaagataaaaatatatcatcatgaacaataaaactgtctgcttacataaacagtaatacaaggggtgttatgagccatattcaacgggaaacgtcttgctcgacgaggccgcgattaaattccaacatggatgctgatttatatgggtataaatgggctcgcgataatgtcgggcaatcaggtgcgacaatctaccgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 5 - DNA, 3715 bp, Artificial Sequence
  pCMV-Mkan DNA plasmid expression vector without stuffer nucleotide sequence cloning site
acatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacagctgcgcatccatcacactggcggccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatggtgtctcaaaatctctgatgttacattgcacaagataaaaatatatcatcatgaacaataaaactgtctgcttacataaacagtaatacaaggggtgttatgagccatattcaacgggaaacgtcttgctcgacgaggccgcgattaaattccaacatggatgctgatttatatgggtataaatgggctcgcgataatgtcgggcaatcaggtgcgacaatctaccgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 6 - DNA, 46 bp, Artificial Sequence
  pMaxVax primer 1
acacatagcgccggcgctagctgagcaaaaggccagcaaaaggcca

SEQ ID NO: 7 - DNA, 60 bp, Artificial Sequence
  pMaxVax primer 2
aactctgtgagacaacagtcataaatgtacagatatcagaccaagtttactcatatatac

SEQ ID NO: 8 - DNA, 39 bp, Artificial Sequence
  pMaxVax primer 3
ggcttctcacagagtggcgcgccgtgtctcaaaatctct

SEQ ID NO: 9 - DNA, 40 bp, Artificial Sequence
  pMaxVax primer 4
ttgctcagctagcgccggcgccgtcccgtcaagtcagcgt

SEQ ID NO: 10 - DNA, 40 bp, Artificial Sequence
  pMaxVax primer 5
agatctgtttaaaccgctgatcagcctcgactgtgccttc

SEQ ID NO: 11 - DNA, 40 bp, Artificial Sequence
  pMaxVax primer 6
acctctaaccactctgtgagaagccatagagcccaccgca

SEQ ID NO: 12 - DNA, 53 bp, Artificial Sequence
  pMaxVax primer 7
ggatccggtacctctagagaattcggcggccgcagatctgtttaaaccgctga

SEQ ID NO: 13 - DNA, 26 bp, Artificial Sequence
  Stuffer nucleotide sequence
atgcagtggaattcggtacctgatca